(19) United States
(12) Patent Application Publication
(10) Pub. No.: US 2026/0284145 A1
(43) Pub. Date: Sep. 24, 2026

(54) ENGINEERED IMMUNE CELLS FOR TREATING CANCER IN COMBINATION WITH IL-2/IL-15 RECEPTOR BETA-GAMMA AGONISTS

(71) Applicants: Sotio Biotech Inc., Boston, MA (US); Sotio Biotech AG, Basel (CH)

(72) Inventors: Amy JENSEN, Boston, MA (US); Emily KUIPER, Boston, MA (US); Kathleen WHITEMAN, Boston, MA (US); Irena ADKINS, Basel (CH)

(21) Appl. No.: 19/474,444

(22) PCT Filed: Apr. 12, 2024
(86) PCT No.: PCT/US2024/024215

§ 371 Date: Oct. 10, 2025

Related U.S. Application Data
(60) Provisional application No. 63/459,432, filed on Apr. 14, 2023.

(30) Foreign Application Priority Data
May 16, 2023 (EP) 23173530.9

(51) Int. Cl. A61K 38/17; A61K 35/76; A61K 38/20; A61K 40/11; A61K 40/31; A61K 40/42; A61P 35/00

(52) U.S. Cl. CPC A61K 38/1793; A61K 35/76; A61K 38/2086; A61K 40/11; A61K 40/31; A61K 40/4251; A61P 35/00

(57) ABSTRACT

A combined therapy for treating cancer in a subject, comprising an immune cell population and an Interleukin-2/Inter-leukin-15 receptor beta gamma (IL-2/IL-15Rβγ) agonist. The immune cell population comprises genetically engineered immune cells expressing or overexpressing a metabolism-regulating polypeptide, which may (a) improves glucose uptake activity; (b) modulates the Krebs cycle; (c) enhances lactate concentration; and/or (d) diverts or redirects glucose metabolites out of a glycolysis pathway.

### CROSS REFERENCE TO RELATED APPLICATIONS

This application claims the benefit of the filing dates of U.S. Provisional Application No. 63/459,432, filed Apr. 14, 2023, and European Patent Application No. 23 173 530.9, filed May 16, 2023, the entire contents of each of which is incorporated by reference herein.

### FIELD OF THE INVENTION

The present invention relates to an immune cell population comprising genetically engineered immune cells expressing or overexpressing a metabolism modulating polypeptide for use in treatment of cancer in combination with an Interleukin-2/Interleukin-15 receptor beta gamma (IL-2/IL-15Rβγ) agonist.

### BACKGROUND OF THE INVENTION

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve various immune cells including NK cells and cytotoxic T cells having reactivity skewed toward cancer cells (Eshhar et al. 1993, Geiger et al. 1999, Brentjens et al. 2003, Cooper et al. 2003, Imai et al. 2004). While cell-based immune therapies have shown promising therapeutic effects, especially in the application to solid tumors they have faced challenges caused by specific characteristics of the tumor microenvironment (TME), which is the cellular environment created via the interaction between malignant tumor cells and non-transformed cells.

Accordingly, there is still a high medical need to improve cell-based immune therapies especially in the field of solid tumor in order bring these promising therapies to patients.

### SUMMARY OF THE INVENTION

It is reported herein that, unexpectedly, the combination of an exemplary IL-2/IL-15Rβγ agonist with an immune cell population comprising immune effector cells genetically engineered to express a metabolism modulating polypeptide led to enhanced anti-tumor activity after the sequential administration in vivo. This allows for novel combination treatments combining immune cell populations and IL-2/IL-15Rβγ agonists for use in the treatment of cancer patients, especially immune cell populations comprising genetically engineered αβ T cells, γδ T cells, a natural killer (NK) cells and/or NKT cells expressing metabolism modulating polypeptides.

Accordingly, provided herein are combined therapies for cancer involving immune cells (e.g., CAR-T cells or BOXR-CAR-T cells as disclosed herein) genetically engineered to express or overexpress a metabolism-regulating polypeptide and Interleukin-2/Interleukin-15 receptor beta gamma (IL-2/IL-15Rβγ) agonist such as those provided herein.

In some aspects, the present disclosure features a method for treating cancer in a subject that involves a combination of (a) an effective amount of an immune cell population and (b) an effective amount of an IL-2/IL-15Rβγ agonist. The immune cell population comprises immune cells that are genetically engineered to express or overexpress a metabolism-regulating polypeptide, for example, (a) a polypeptide that improves glucose uptake activity; (b) a polypeptide that modulates the Krebs cycle; (c) a polypeptide that enhances lactate concentration; and/or (d) a polypeptide that diverts or redirects glucose metabolites out of a glycolysis pathway.

In some embodiments, the method provided herein comprises administering to a subject in need of the treatment (a) an effective amount of the immune cell population described herein and (b) an effective amount of the IL-2/IL-15Rβγ agonist as also disclosed herein. In other embodiments, the method provided herein comprises administering to a subject in need of the treatment an effective amount of the immune cell population of (a), wherein the subject has received treatment or is on treatment of the IL-2/IL-15Rβγ agonist of (b). In yet other embodiments, the method provided herein comprises administering to a subject in need of the treatment an effective amount of the IL-2/IL-15Rβγ agonist of (b), wherein the subject has received treatment or is on treatment of the immune cell population. Such a treatment can increase viability of the immune cell population and/or increase the anti-tumor cytotoxicity of the immune cells.

In some instances, the metabolism-regulating polypeptide improves glucose uptake activity, e.g., a glucose importation polypeptide. Examples include, but are not limited to, a glucose transporter (GLUT) or a sodium-glucose cotransporter (SGLT). In some examples, the glucose importation polypeptide is a GLUT, e.g., GLUT1, GLUT3, GLUT1 S226D, GLUT8, GLUT8 L12A L13A, GLUT11, GLUT7, or GLUT4. In other examples, the glucose importation polypeptide is a SGLT, e.g., SGLT1 or SGLT2.

In some instances, the metabolism-regulating polypeptide modulates the Krebs cycle. In some examples, such a polypeptide can be an enzyme that catalyzes a reaction in the Krebs cycle (e.g., isocitrate dehydrogenase (IDH), malate dehydrogenase (MDH), or phosphoglycerate dehydrogenase (PHGDH)). In other examples, the polypeptide that modulates the Krebs cycle is an enzyme that uses a Krebs cycle metabolite as a substrate, e.g., glutamic-oxaloacetic transaminase (GOT) or phosphoenolpyruvate carboxykinase 1 (PCK1). In yet other examples, the polypeptide that modulates the Krebs cycle is an enzyme that converts a precursor to a Krebs cycle metabolite, e.g., phosphoserine aminotransferase (PSAT1), glutamate dehydrogenase (GDH1), glutamate-pyruvate transaminase 1 (GPT1), or glutaminase (GLS). In still other examples, the metabolism-regulating polypeptide enhances lactate concentration. Examples include monocarboxylate transporter (MCT) (e.g., MCT1, MCT2, or MCT4), an enzyme involved in lactate synthesis (e.g., lactate dehydrogenase A (LDHA)), or a polypeptide that inhibits a pathway that competes for lactate-synthesis substrates (e.g., pyruvate dehydrogenase kinase 1 (PDK1)). In yet other examples, the metabolism-regulating polypeptide can divert or redirect glucose metabolites out of a glycolysis pathway. Examples include pyruvate kinase muscle isozyme M2 (PKM2), glutamine-fructose-6-phosphate aminotransferase 1 (GFPT1), and TP53-inducible glycolysis and apoptosis regulator (TIGAR). In some examples, the expression or overexpression of the metabolism-regulating polypeptide disclosed herein in the immune cells can lead to an at least 70% increase of proliferation of the immune cell population when compared to the native immune cell population of the same type.

In some instances, the immune cell population comprises immune cells of B cells, αβ T cells, yd T cells, natural killer (NK) cells or NKT cells. In some instances, the immune cells may comprise a combination of any of B cells, αβ T cells, γδ T cells, natural killer (NK) cells, and NKT cells.

The immune cells are genetically modified to express or overexpress the metabolism-regulating polypeptide. In some instances, the immune cells are autologous to the subject. Alternatively, the immune cells are allogenic to the subject. In some instances, the immune cells are expanded ex vivo prior to administration. For examples, the immune cells can be activated ex vivo in the presence of one or more of 4-1BB ligand, anti-4-1BB antibody, anti-CD3 antibody, anti-CD28 antibody, IL-15, anti-IL-15 receptor antibody, IL-2, IL-7, IL-12, IL-21 and K562 cells, and an engineered artificial stimulatory cell or particle prior to administration.

In some instances, the immune cells are derived from one or more cell lines. In other instances, the immune cells are derived from peripheral blood mononuclear cells (PBMC), hematopoietic stem cells (HSCs), cord blood stem cells or induced pluripotent stem cells (iPSCs). In yet other instances, the immune cells are from tumor tissue, for example, comprising tumor-infiltrating lymphocytes (TILs).

In some embodiments, the immune cell population described herein may comprise genetically engineered immune cell expressing a chimeric receptor polypeptide. The chimeric receptor polypeptide comprises: (a) an extracellular target binding domain; (b) a transmembrane domain; and (c) a cytoplasmic signaling domain.

In some examples, the chimeric receptor polypeptide is a chimeric antigen receptor (CAR) polypeptide or a T-Cell Receptor (TCR) polypeptide. The CAR polypeptide may comprise an extracellular antigen binding domain, which may bind to a tumor antigen. In some instances, the extracellular antigen binding domain may be a single chain variable fragment (scFv). In other instances, the extracellular antigen binding may be a single domain antibody.

In some embodiments, the transmembrane domain (b) of any of the chimeric receptor polypeptides provided herein may be of a membrane protein. Examples include: CD8α, CD8β, 4-1BB, CD28, CD34, CD4, FcεRIγ, CD16α, OX40, CD3ζ, CD3ε, CD3γ, CD3δ, TCRα, CD32, CD64, VEGFR2, FAS, NKG2D, NKp44, NKp46, and FGFR2B.

In some embodiments, the cytoplasmic signaling domain of (c) of any of the chimeric receptor polypeptides provided herein may comprise a cytoplasmic domain of CD3ζ. Alternatively, it may comprise a cytoplasmic domain of FcεRIγ.

Any of the chimeric receptor polypeptides provided herein may further comprise a signal peptide at its N-terminus. In some examples, the chimeric receptor polypeptide may further comprise a hinge domain, which may be located at the C-terminus of (a) and the N-terminus of (b). Alternatively, the chimeric receptor polypeptide may be free of a hinge domain. In some examples, the chimeric receptor polypeptide may further comprise at least one co-stimulatory signaling domain. Examples include, but are not limited to, a co-stimulatory domain from CD8α, CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL, or any functional variant thereof. Alternatively, the chimeric receptor polypeptide may be free of a co-stimulatory signaling domain. In other instances, the chimeric receptor polypeptide may comprise a cytoplasmic signaling domain that comprises an immunoreceptor tyrosine-based activation motif (ITAM).

In some instances, the IL-2/IL-15Rβγ agonist can be an IL-15/IL-15Rα complex comprising: (i) a first polypeptide comprising the amino acid sequence of the interleukin 15 (IL-15) or a derivative thereof; and (ii) a second polypeptide comprising the amino acid sequence of the sushi domain of an interleukin 15-receptor alpha (IL-15Rα) or a derivative thereof.

In some examples, the first polypeptide comprises the amino acid sequence of human IL-15 or a derivative thereof. In some instances, the IL-15 derivative may have an amino acid sequence at least 92.5% identical with the amino acid of SEQ ID NO: 74. In one specific example, the first polypeptide comprises the amino acid sequence of SEQ ID NO: 74. Alternatively or in addition, the second polypeptide comprises the sushi domain of the human IL-15Rα or a derivative thereof. In some instances, the sushi domain of the IL-15Rα derivative may have an amino acid sequence at least 92% identical with the amino acid sequence of SEQ ID NO: 76 or SEQ ID NO: 77. In one specific example, the second polypeptide comprises the amino acid sequence of SEQ ID NO: 76. In another specific example, the second polypeptide comprises the amino acid sequence of SEQ ID NO: 77.

In some instances, the IL-15/IL-15Rα complex can be a fusion protein comprising the first and second polypeptides. For example, the first polypeptide may be located in a C-terminal position relative to the second polypeptide in the fusion protein. Alternatively or in addition, the first and second polypeptides may be separated by a peptide linker.

In some instances, the IL-2/IL-15Rβγ agonist may comprise: (i) a first polypeptide comprising the amino acid sequence of an IL-15 derivative having at least 0.1% of the activity of human IL-15 on the proliferation induction of kit225 cell line, and (ii) a second polypeptide comprising the amino acid sequence of the sushi domain of IL-15Rα or derivatives thereof having at least 10% of the binding activity of the sushi domain of human IL-15Rα to human IL-15. In some examples, the IL-15 derivative has at least 25% (e.g., at least 50%) of the activity of human IL-15 on the proliferation induction of kit225 cell line. Such an IL-15 derivative may have an amino acid sequence at least 92.5% (e.g., at least 96%, at least 98.5% or at least 99%) identical with SEQ ID NO: 74. Alternatively or in addition, the derivative of the sushi domain of IL-15Rα has at least 25% (e.g., at least 50%) of the binding activity of the sushi domain of human IL-15Rα. Such a sushi domain derivative may have an amino acid sequence at least 92.5% (e.g., at least 96% or at least 98%) identical with an amino acid sequence selected in the group comprising SEQ ID NO: 76 or SEQ ID NO: 77.

In one specific example, the IL-2/IL-15Rβγ agonist comprises the sequence of SEQ ID NO: 79.

In some instances, an effective dose of the immune cell population and an effective dose of the agonist are administered sequentially to the subject. In some examples, the agonist is administered to the subject following a cyclical regimen as exemplified below:
(a) a first period of “x” days during which the agonist is administered at a daily dose on “y” consecutive days at the beginning of the first period followed by “x-y” days without administration of the agonist,
wherein “x” is 7, 14 or 21 days, preferably 7 days and “y” is 1, 2, 3 or 4 consecutive days;
(b) repeating the first period at least once; and
(c) a second period of “z” days without administration of the agonist wherein z is 7, 14 or, 21 days, preferably 7 or 14 days.

In some examples, the agonist can be administered to the subject at a daily dose of 4.4 pmol/kg to 2200 μmol/kg. In one example, the agonist can be administered to the subject at a daily dose of 22 μmol/kg to 660 μmol/kg. In a specific example, the agonist can be administered to the subject at a daily dose of 220 μmol/kg to 530 μmol/kg.

The administration of the IL-2/IL-15Rβγ agonist provided herein may results in one or more of the following effects:
(a) an increase of the % of Ki-67⁺ NK of total NK cells in comparison to no administration of the conjugate, and wherein the administration of the conjugate after at least one repetition of the first period (step (b)) results in a Ki-67⁺ NK cell level that is at least 70% of the of the Ki-67⁺ NK cells;
(b) maintenance of NK cell numbers or preferably an increase of NK cell numbers to at least 110% as compared to no administration of conjugate after at least one repetition of the first period (step (b)); and/or
(c) NK cell numbers of at least 1.1×10³ NK cells/μl after at least one repetition of the first period (step (b)).

Alternatively or in addition, any of the immune cell populations disclosed herein is administered to the subject at a dose of at least about 1×10⁴ immune cells/kg. In some examples, the dose is from about 1×10⁴ to about 1×10¹² cells/kg.

Further, the present disclosure provides a method for treating cancer, involving the combination of a viral particle capable of transducing immune cells such as T cells as disclosed herein and any of the IL-2/IL-15Rβγ agonists provided herein. The viral particle comprises a vector genome comprising a polynucleotide sequence encoding any of the chimeric receptor polypeptides provided herein. In some instances, the vector genome may further comprise a polynucleotide sequence encoding any of the metabolism-regulating polypeptide also provided herein. In some instances, the viral particle is a retroviral particle. In other instances, the viral particle can be a lentiviral particle.

In some embodiments, the method provided above comprises administering to a subject in need of the treatment an effective amount of the viral particle and an effective amount of the IL-2/IL-15Rβγ agonists. In some examples, the viral particle and the IL-2/IL-15Rβγ agonists are administered sequentially. In other embodiments, the method provided herein comprises administering to a subject in need of the treatment an effective amount of the viral particle, wherein the subject has received treatment or is on treatment of the IL-2/IL-15Rβγ agonists. In yet other embodiments, the method provided herein comprises administering to a subject in need of the treatment an effective amount of the IL-2/IL-15Rβγ agonist, wherein the subject has received treatment or is on treatment of the viral particle. In some examples, the viral particle is for intravenous, intraperitoneal, subcutaneous or intranodal injection.

In addition, the present disclosure provides a method for treating cancer, involving a combination of a nucleic acid encoding a chimeric receptor polypeptide as disclosed herein and any of the IL-2/IL-15Rβγ agonists as also disclosed herein. In some instances, the nucleic acid can be a vector. In some instances, the nucleic acid (e.g., the vector) may form a non-viral in vivo transduction system, which may be a lipid nanoparticle, a liposome, a polymeric nanoparticle or an inorganic nanoparticle encapsulating the vector. In some instances, the nucleic acid or the non-viral transduction system and the conjugate may be administered to a subject in need of the treatment sequentially.

Any of the cancer treatment methods disclosed herein may further comprise one or more of the following:
(a) a lymphocyte reduction treatment, which preferably comprises cyclophosphamide and/or fludarabine;
(b) a therapeutic agent; and/or
(c) a further anti-tumor treatment.

In some embodiments, the subject to be treated by any of the cancer treatment methods provided herein can be a human patient suffering from a cancer. In some examples, the human patient may have a solid tumor (e.g., carcinoma, lymphoma, sarcoma and blastoma). Alternatively, the human patient may have a liquid tumor. In some instances, the human patient may have a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, mesothelioma, pancreatic cancer, head and neck cancer, retinoblastoma, glioma, glioblastoma, liver cancer, and thyroid cancer. In some examples, the cancer of B-cell origin is Hodgkin lymphoma or non-Hodgkin lymphoma. In some instances, the tumor has a low-glucose tumor microenvironment.

Also within the scope of the present disclosure are methods for inhibiting and/or killing tumor cells in a subject, using any of the combinations of (a) immune cells, viral particles, and/or nucleic acid, and (b) any of the conjugates provided herein.

Other exemplary embodiments provided herein include:

A method for inhibiting and/or killing tumor cells in a subject, the method comprising administering to a subject in need thereof the immune cell population disclosed herein in combination with any of the IL-2/IL-15Rβγ agonists as also disclosed herein. In some instances, a therapeutically effective dose of the immune cell population and a therapeutically effective dose of the IL-2/IL-15Rβγ agonist can be administered sequentially. A method for increasing the viability of a previously administered immune cell population in the tumor microenvironment of a subject by subsequently administering an IL-2/IL-15Rβγ agonist as disclosed herein.

A method for increasing the anti-tumor cytotoxicity of a previously administered immune cell population as disclosed herein in the tumor microenvironment of a subject by subsequently administering an IL-2/IL-15Rβγ agonist as also disclosed herein. A method for inhibiting and/or killing tumor cells in a subject, the method comprising: administering to a subject in need thereof a therapeutically effective amount of viral particles, a nucleic acid (e.g., a vector), or a non-viral transduction system as disclosed herein in combination with the IL-2/IL-15Rβγ agonist as also disclosed herein. The viral particles, the nucleic acid such as the vector or the non-viral transduction system are suitable for transducing immune cells in vivo. In some instances, the therapeutically effective dose of the viral particles, the nucleic acid such as the vector or the non-viral transduction system, and a therapeutically effective dose of the IL-2/IL-15Rβγ agonist are administered sequentially.

A method for increasing the viability of in vivo transduced immune cells in the tumor microenvironment after administration of viral particles, a nucleic acid such as a vector, or a non-viral transduction system as disclosed herein in a subject by subsequently administering an IL-2/IL-15Rβγ agonist as also disclosed herein.

A method for increasing the anti-tumor cytotoxicity of in vivo transduced immune cells in the tumor microenvironment after administration of viral particles, a nucleic acid such as a vector, or a non-viral transduction system as disclosed herein in a subject by subsequently administering the IL-2/IL-15Rβγ agonist are also disclosed herein.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1F include diagrams showing SOT101 concentration dependent proliferation of activated T cell products. FIGS. 1A-1C show proliferation of activated T cell products in the presence of SOT101. T cells were seeded in 96-well U bottom plate and treated with SOT101. At day 3, 5, or 7, cells were stained with for cell markers and intracellularly stained for the proliferation marker Ki67. The percentage of Ki67+ T cells is shown in dependence of indicated SOT101 concentration. FIG. 1A: Untransduced T cells (UTD). FIG. 1B: CAR T cells directed to ROR-1 (CAR). FIG. 1C: GOT2 expressing CAR T cells directed to ROR-1 (BOXR). FIGS. 1D-1F show proliferation of activated T cell products in the presence of SOT101 in comparison with 0.2 nM IL-2. FIG. 1D: Untransduced T cells. FIG. 1E: CAR-T cells. FIG. 1F: BOXR T cells. Cells were gated on CD8⁺, mean and SEM are of 3 donors.

FIGS. 2A-2N include diagrams showing analysis of T cell memory subsets after SOT101 treatment. Gating strategy-stem cell like T cell (T_{scm}): CD45RA⁺, CCR7⁺; naïve central memory T cell (T_{CM}): CD45RA, CCR7⁺; effector memory T cell (T_{EM}): CD45RA⁻, CCR7⁻; effector memory cells re-expressing CD45RA (T_{emRA}): CD45RA⁺, CCR7⁻. Analysis of memory subsets provided as % of CAR-expressing CD8⁺ T cells for indicated concentrations of SOT101 or IL-2 of CAR-T cells (FIGS. 2A-2C) GOT2 BOXR-T cells (FIGS. 2D-2F) after 3, 5, or 7 days of culture. For each column from bottom to top: T_{CM}-T_{EM}-T_{emRA}-T_{scm}. Comparison of CAR-expressing CD8⁺ T cells of CAR-T cells (FIGS. 2G-2J) GOT2 BOXR-T cells (FIGS. 2K-2N) of 0.1 nM SOT101 or 0.2 nM IL-2 treatment provided as % of indicated T cell memory subset. Data shown as mean and SEM are of 3 donors.

FIGS. 3A-3F include diagrams showing cytotoxicity of T cell products with SOT101 or IL-2 pre-treatment. Indicated T cells were treated with 0.1 nM SOT101 or IL-2 for 3 days then applied to A549 ROR-1 positive tumor cells (FIGS. 3A-3C) or Hep3b ROR-1 negative tumor cells (FIGS. 3D-3F). UTD for untransduced T cells, CAR for T cells transduced with the CAR construct, GOT2 BOXR or BOXR for T cells transduced with CAR and GOT2. Tumor cells were counted with time. Increase in T cell cytotoxicity with SOT101 or IL-2 treatment compared to no treatment.

FIGS. 4A-4D include diagrams showing memory phenotype of T cell products post tumor cell exposure. T cells were treated with 0.1 nM SOT101 or IL-2 for 3 days then co-cultured with A549 ROR-1⁺ positive tumor cells or Hep3b ROR-1-tumor cells for 4 days after which cells were analyzed for T cell differentiation markers as outlined for FIG. 2. % of CAR⁺/CD8⁺ T cells according to the T_{CM}, T_{SCM}, T_{emRA} or T_{EM} phenotype (see gating strategy) after no stimulation (No) or stimulation with 0.1 nM SOT101 or IL-2 for co-culturing with ROR-1⁺ A549 tumor cells (FIG. 4A), or co-culturing with ROR-1-Hep3b tumor cells (FIG. 4B). % of CAR⁺ CD8⁺ T_{EM} population (CD45RA⁻, CCR7⁻) is shown for CAR T cells (FIG. 4C) and GOT2 BOXR T cells (FIG. 4D) for both A549 and Hep3b tumor cells.

FIGS. 5A-5G include diagrams showing that SOT101 improves CAR T cell or BOXR CAR T cell in vivo efficacy in a human renal cell carcinoma mouse model. Mice implanted with CAKI-1 cell line xenografts were untreated (control) or treated with 3×10⁶ CAR T cells or BOXR CAR T cells. Dashed vertical lines indicate SOT101 dosage. FIG. 5A: CAR T cells or CAR T cells in combination with 15 μg/kg per dose of SOT101 at day 7, 8, 14, and 15 post T cell infusion. FIG. 5B: BOXR CAR T cells or BOXR CAR T cells in combination with 15 μg/kg per dose SOT101 at day 7, 8, 14, and 15. Individual mouse efficacy curves are shown in panels. GOT2 is the BOXR transgene within the BOXR CAR construct. FIG. 5C: Tumor volume curves of individual mice within the untreated group at day 7, 8, 14, and 15 post T cell infusion. FIG. 5D: Tumor volume curves of individual mice within the treated group of CAR T cells at day 7, 8, 14, and 15 post T cell infusion. FIG. 5E: Tumor volume curves of individual mice within the treated group of CAR T cells and SOT101 at day 7, 8, 14, and 15 post T cell infusion. FIG. 5F: Tumor volume curves of individual mice within the treated group of BOXR CAR T cells at day 7, 8, 14, and 1. FIG. 5G: Tumor volume curves of individual mice within the treated group of BOXR T cells in combination with SOT101 at day 7, 8, 14, and 15.

FIGS. 6A-6C include diagrams showing peripheral blood T cell expansion upon SOT101 treatment. Enumeration of T cells from retro-orbital bleeds. GOT2 is the BOXR transgene within the BOXR CAR construct. FIG. 6A: T cell counts from CAR T cell and BOXR CAR T cell treated mice with no SOT101 treatment at day 7, 8, 14, and 15 post T cell infusion. FIG. 6B: T cell counts from mice treated with CAR T cells and SOT101 at day 7, 8, 14, and 15 post T cell infusion. FIG. 6C: T cell counts from mice treated with BOXR CAR T cells in combination with SOT101 at day 7, 8, 14, and 15. SOT101 improves T cell peripheral expansion, with maximum expansion observed at Day 21.

FIGS. 7A-7B include diagrams showing that SOT101 improves CAR T cell or BOXR CAR T cell efficacy in a human non-small cell lung cancer mouse model. Mice implanted with NCI-H1975 cell line xenografts were untreated or treated with 3×10⁶ CAR T cells or BOXR CAR T cells. Dashed vertical lines indicate SOT101 dosage. FIG. 7A: CAR T cells or CAR T cells in combination with SOT101 at day 7, 8, 14, and 15 post T cell infusion at day 7, 8, 14, and 15. GOT2 is the BOXR transgene within the BOXR CAR construct. FIG. 7B: BOXR CAR T cells, or BOXR CAR T cells in combination with SOT101 at day 7, 8, 14, and 15. GOT2 is the BOXR transgene within the BOXR CAR construct.

### DEFINITIONS, ABBREVIATIONS AND ACRONYMS

“IL-2/IL-15Rβγ agonist” refers to a molecule or complex of an IL-2 or IL-2 derivative, or an IL-15 or IL-15 derivative targeting the mid-affinity IL-2/IL-15Rβγ and having a decreased or abandoned binding of the IL-2Rα or IL-15Rα. Decreased binding in this context means at least 50%, preferably at least 80% and especially at least 90% decreased binding to the respective Receptor & compared to the wild-type IL-15 or IL-2, respectively. As described and exemplified below, decreased or abandoned binding of IL-15 to the respective IL-15Rα may be mediated by forming a complex (covalent or non-covalent) with an IL-15Rα derivative, by mutations in the IL-15 leading to a decreased or abandoned binding, or by site-specific PEGylation or other post-translational modification of the IL-15 leading to a decreased or abandoned binding. Similarly, decreased or abandoned binding of IL-2 to the respective IL-2Rα may be mediated by mutations in the IL-2 leading to a decreased or abandoned binding, or by site-specific PEGylation or other post-translational modification of the IL-2 leading to a decreased or abandoned binding. Decreased binding may also be expressed as the ratio between the binding of the Rβγ versus the binding of the Rαβγ, i.e., for IL-2-based compounds the ratio of binding affinity ratio

$$\frac{KD ( IL - 2 R bg )}{KD ( IL - 2 R a b g )}$$

is reduced at least 5fold, preferably at least 10fold, more preferably at least 100fold compared to wildtype human IL-2, and for IL-15-based compounds, the binding affinity ration

$$\frac{KD ( IL - 15 Rbg )}{KD ( IL - 15 Rabg )}$$

is reduced at least 5fold, preferably at least 10fold, more preferably at least 100 fold compared to wildtype human IL-15. KD stands for equilibrium dissociation constant and may be determined by Biacore SPRC technology. Such compounds are hereinafter referred to as IL-2/IL-15Rβγ agonists.

Accordingly, the IL-2/IL-15Rβγ agonists have no or decreased binding to cells expressing the IL-2Rαβγ or IL-15Rαβγ with respectively improved properties as outlined above. Examples are IL-15 bound to at least the sushi domain of the IL-15Rα having the advantage of not being dependent on trans-presentation or cell-cell interaction, and of a longer in vivo half-life due to the increased size of the molecule, which have been shown to be significantly more potent that native IL-15 in vitro and in vivo (Robinson and Schluns 2017). Besides complexes comprising the sushi domain of the IL-2Rα or the IL-15Rα, this can be achieved by mutated or chemically modified IL-2 or IL-15, which have a markedly reduced or timely delayed binding to the IL-2Rα or IL-15Rα without affecting the binding to the IL-2/15Rβ and γ_{c} receptor.

“Interleukin-2”, “IL-2” or “IL2” refers to the human cytokine as described by NCBI Reference Sequence AAB46883.1 or UniProt ID P60568 (SEQ ID NO: 49). Its precursor protein has 153 amino acids, having a 20-aa peptide leader and resulting in a 133-aa mature protein. Its mRNA is described by NCBI GenBank Reference S82692.1.

“IL-2 derivative” refers to a protein having a percentage of identity of at least 92%, preferably of at least 96%, more preferably of at least 98%, and most preferably of at least 99% with the amino acid sequence of the mature human IL-2 (SEQ ID NO: 50). Preferably, an IL-2 derivative has at least about 0.1% of the activity of human IL-2, preferably at least 1%, more preferably at least 10%, more preferably at least 25%, even more preferably at least 50%, and most preferably at least 80%, as determined by a lymphocyte proliferation bioassay. As interleukins are extremely potent molecules, even such low activities as 0.1% of human IL-2 may still be sufficiently potent, especially if dosed higher or if an extended half-life compensates for the loss of activity. Its activity is expresses in International Units as established by the World Health Organization 1^{st} International Standard for Interleukin-2 (human), replaced by the 2^{nd} International Standard (Gearing and Thorpe 1988, Wadhwa et al. 2013). The relationship between potency and protein mass is as follows: 18 million IU PROLEUKIN=1.1 mg protein. As described above, mutations (substitutions) may be introduced in order to specifically link PEG to IL-2 for extending the half-life as done for THOR-707 (Joseph et al. 2019) (WO2019/028419A1) or for modifying the binding properties of the molecule, e.g. reduce the binding to the IL-2α receptor as done for IL2v (Klein et al. 2013, Bacac et al. 2016) (WO 2012/107417A1) by mutation of L72, F42 and/or Y45, especially F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In some examples, the mutations include F42A, Y45A and L72G. Various other mutations of IL-2 have been described: R38W for reducing toxicity (Hu et al. 2003) due to reduction of the vasopermeability activity (US 2003/0124678); N88R for enhancing selectivity for T cells over NK cells (Shanafelt et al. 2000); R38A and F42K for reducing the secretion of proinflammatory cytokines from NK cells ((Heaton et al. 1993) (U.S. Pat. No. 5,229,109); D20T, N88R and Q126D for reducing VLS (US 2007/0036752); R38W and F42K for reducing interaction with CD25 and activation of T_{reg} cells for enhancing efficacy (WO 2008/003473); and additional mutations may be introduced such as T3A for avoiding aggregation and C125A for abolishing O-glycosylation (Klein et al. 2017). Other mutations or combinations of the above may be generated by genetic engineering methods and are well known in the art. Amino acid numbers refer to the mature IL-2 sequence of 133 amino acids.

“Interleukin-15”, “IL-15” or “IL15” refers to the human cytokine as described by NCBI Reference Sequence NP_000576.1 or UniProt ID P40933 (SEQ ID NO: 54). Its precursor protein has 162 amino acids, having a long 48-aa peptide leader and resulting in a 114-aa mature protein (SEQ ID NO: 55). Its mRNA, complete coding sequence is described by NCBI GenBank Reference U14407.1.

“IL-15 derivative” or “derivative of IL-15” refers to a protein having a percentage of identity of at least 92%, preferably of at least 96%, more preferably of at least 98%, and most preferably of at least 99% with the amino acid sequence of the mature human IL-15 (114 aa) (SEQ ID NO: 55). In one embodiment, an IL-15 derivative has at least 0.01% activity of human IL-15. Preferably, an IL-15 derivative has at least 0.1% of the activity of human IL-15, preferably at least 1%, more preferably at least 10%, more preferably at least 25%, even more preferably at least 50%, and most preferably at least 80%. Interleukins are extremely potent but short-lived molecules, even such low activities as 0.01% or 0.1% of human IL-15 may still be sufficiently potent, especially if dosed higher or if an extended half-life compensates for the loss of activity. A reduced binding of a mutant IL-15 or a mutant IL-15/IL-15Rα complex to the IL-2/IL-15Rβγ receptor results in a lower target-mediated drug deposition and therefore to a longer in vivo half-life. Accordingly, the longer in vivo half-life with its increased AUC of the complex compensates for the lower in vitro activity.

For human IL-15, a plethora of mutations has been described in order to achieve various defined changes to the molecule: D8N, D8A, D61A, N65D, N65A, Q108R for reducing binding to the IL-15Rβγ_{c} receptors (WO 2008/143794A1); N72D as an activating mutation (in ALT-803); NID, N4D, D8N, D30N, D61N, E64Q, N65D, and Q108E to reduce the proliferative activity (US 2018/0118805); L44D, E46K, L47D, V49D, 150D, L66D, L66E, I67D, and I67E for reducing binding to the IL-15Rα (WO 2016/142314A1); N65K and L69R for abrogating the binding of IL-15Rβ (WO 2014/207173A1); Q101D and Q108D for inhibiting the function of IL-15 (WO 2006/020849A2); S7Y, S7A, K10A, K11A for reducing IL-15Rβ binding (Ring et al. 2012); L45, S51, L52 substituted by D, E, K or R and E64, 168, L69 and N65 replaced by D, E, R or K for increasing the binding to the IL-15Rα (WO 2005/085282A1); N71 is replaced by S, A or N, N72 by S, A or N, N77 by Q, S, K, A or E and N78 by S, A or G for reducing deamidation (WO 2009/135031A1); WO 2016/060996A2 defines specific regions of IL-15 as being suitable for substitutions (see para. 0020, 0035, 00120 and 00130) and specifically provides guidance, how to identify potential substitutions for providing an anchor for a PEG or other modifications (see para. 0021); Q108D with increased affinity for CD122 and impaired recruitment of CD132 for inhibiting IL-2 and IL-15 effector functions and N65K for abrogating CD122 affinity (WO 2017/046200A1); N1D, N4D, D8N, D30N, D61N, E64Q, N65D, and Q108E for gradually reducing the activity of the respective IL-15/IL-15Rα complex regarding activating of NK cells and CD8 T cells (see FIG. 51, WO 2018/071918A1, WO 2018/071919A1). Additionally or alternatively, the artisan can easily make conservative amino acid substitutions. IL-15 muteins can be generated by standard genetic engineering methods and are well known in the art, e.g., from WO 2005/085282, US 2006/0057680, WO 2008/143794, WO 2009/135031, WO 2014/207173, WO 2016/142314, WO 2016/060996, WO 2017/046200, WO 2018/071918, WO 2018/071919, US 2018/0118805. IL-15 derivatives may further be generated by chemical modification as known in the art, e.g., by PEGylation or other posttranslational modifications (see WO 2017/112528A2, WO 2009/135031).

The activity of both IL-2 and IL-15 can be determined by induction of proliferation of kit225 cells as described by Hori et al. (1987). Preferably, methods such as colorimetry or fluorescence are used to determine proliferation activation due to IL-2 or IL-15 stimulation, as for example described by Soman et al. using CTLL-2 cells (Soman et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

The IL-2 derivative may have at least 0.01%, preferably at least 0.1% of the activity of human IL-2, preferably at least 1%, more preferably at least 10%, even more preferably at least 25%, even more preferably at least 50%, and most preferably at least 80%. The IL-15 derivative may have at least 0.01%, preferably at least 0.1% of the activity of human IL-15, more preferably at least 1%, even more preferably at least 10%, even more preferably at least 25%, even more preferably at least 50%, and most preferably at least 80%.

“IL-2Rα” refers to the human IL-2 receptor α or CD25.

“Receptor α” refers to the IL-2Rα or IL-15Rα.

“IL-15Rα” refers to the human IL-15 receptor α or CD215 as described by NCBI Reference Sequence AAI21142.1 or UniProt ID Q13261 (SEQ ID NO: 56). Its precursor protein has 267 amino acids, having a 30-aa peptide leader and resulting in a 237-aa mature protein. Its mRNA is described by NCBI GenBank Reference HQ401283.1. The IL-15Rα sushi domain (or IL-15R (sushi, SEQ ID NO: 57) is the domain of IL-15Rα which is essential for binding to IL-15 (Wei et al. 2001). The sushi+ fragment (SEQ ID NO: 58) comprising the sushi domain and part of the hinge region, defined as the fourteen amino acids which are located after the sushi domain of this IL-15Rα, in a C-terminal position relative to said sushi domain, i.e., said IL-15Rα hinge region begins at the first amino acid after said (C4) cysteine residue, and ends at the fourteenth amino acid (counting in the standard “from N-terminal to C-terminal” orientation). The sushi+ fragment reconstitutes full binding activity to IL-15 (WO 2007/046006).

“Receptor α” refers to the IL-2Rα and/or IL-15Rα.

“IL-15Rα derivative” refers to a polypeptide comprising an amino acid sequence having a percentage of identity of at least 92%, preferably of at least 96%, more preferably of at least 98%, and even more preferably of at least 99%, and most preferably 100% identical with the amino acid sequence of the sushi domain of human IL-15Rα (SEQ ID NO: 57) and, preferably of the sushi+ domain of human IL-15Rα (SEQ ID NO: 58). Preferably, the IL-15Rα derivative is a N- and C-terminally truncated polypeptide, whereas the signal peptide (amino acids 1-30 of SEQ ID NO: 56) is deleted and the transmembrane domain and the intracytoplasmic part of IL-15Rα is deleted (amino acids 210 to 267 of SEQ ID NO: 56). Accordingly, preferred IL-15Rα derivatives comprise at least the sushi domain (aa 33-93) but do not extend beyond the extracellular part of the mature IL-15Rα being amino acids 31-209 of SEQ ID NO: 56. Specific preferred IL-15Rα derivatives are the sushi domain of IL-15Rα (SEQ ID NO: 57), the sushi+ domain of IL-15Rα (SEQ ID NO: 58) and a soluble form of IL-15Rα (from amino acids 31 to either of amino acids 172, 197, 198, 199, 200, 201, 202, 203, 204 or 205 of SEQ ID NO: 56, see WO 2014/066527, (Giron-Michel et al. 2005)). Within the limits provided by this definition, the IL-15Rα derivative may include natural occurring or introduced mutations. Natural variants and alternative sequences are, e.g., described in the UniProtKB entry Q13261 (uniprot.org/uniprot/Q13261). An S40C substitution has been identified in order to enable an artificial disulfide bond with an IL-15 derivative having a L52C substitution (in WO 2016/095642 and Hu et al. (2018)). Further, the artisan can easily identify less conserved amino acids between mammalian IL-15Rα homologs or even primate IL-15Rα homologs in order to generate derivatives which are still functional. Respective sequences of mammalian IL-15Rα homologs are described in WO 2007/046006, page 18 and 19. Additionally or alternatively, the artisan can easily make conservative amino acid substitutions.

In some embodiments, an IL-15Rα derivative has at least 10% of the binding activity of the human sushi domain to human IL-15, e.g. as determined in (Wei et al. 2001), more preferably at least 25%, even more preferably at least 50%, and most preferably at least 80%.

IL-2Rα derivatives may be generated in analogy the IL-15Rα derivatives.

“IL-2Rβ” refers to the Interleukin-2 receptor β or CD122.

“IL-2Rγ” refers to the common cytokine receptor γ or γ_{c} or CD132, shared by IL-4, IL-7, IL-9, IL-15 and IL-21.

“RLI-15” refers to an IL-15/IL-15Rα complex being a receptor-linker-interleukin (from N- to C-terminus; “RLI”) fusion protein of the human IL-15Rα sushi+ fragment with the human IL-15. Suitable linkers are flexible with low immunogenicity, examples are described in WO 2007/046006 and WO 2012/175222. The sushi domain or fragment of human IL-15Rα has the sequence as described by SEQ ID NO: 57 from the first to the fourth conserved cysteine, optionally extended N-terminally by T or IT and C-terminally by I. The sushi+ fragment of human IL-15Rα has the sequence as described by SEQ ID NO: 58, which additionally comprises part of the hinge region.

“SOT101” (also “nanrilkefusp alfa”, “RLI2” or “SO-C101”) refers to an IL-15/IL-15Rα complex being a receptor-linker-interleukin fusion protein of the human IL-15Rα sushi+ fragment with the human IL-15 represented by (SEQ ID NO: 60) using the linker with the SEQ ID NO: 59.

“ALT-803” (also N803) refers to an IL-15/IL-15Rα complex of Altor BioScience Corp., which is a complex containing 2 molecules of an optimized amino acid-substituted (N72D) human IL-15 “superagonist”, 2 molecules of the human IL-15α receptor “sushi” domain fused to a dimeric human IgG1 Fc that confers stability and prolongs the half-life of the IL-15_{N72D}:IL-15Rαₛᵤₛₕᵢ-Fc complex (see for example US 2017/0088597).

“Heterodimeric IL-15:IL-Rα”, “hetIL-15” or “NIZ985” refer to an IL-15/IL-15Rα complex of Novartis which resembles the IL-15, which circulates as a stable molecular complex with the soluble IL-15Rα, which is a recombinantly co-expressed, non-covalent complex of human IL-15 and the soluble human IL-15Rα (sIL-15Rα), i.e. 170 amino acids of IL-15Rα without the signal peptide and the transmembrane and cytoplasmic domain (Thaysen-Andersen et al. 2016, see e.g. table 1).

“NKTR-214” refers to an IL-2/IL-15Rβγ agonist based on IL-2, being a biologic prodrug consisting of IL-2 bound by 6 releasable polyethylene glycol (PEG) chains (WO 2012/065086A1). The presence of multiple PEG chains creates an inactive prodrug, which prevents rapid systemic immune activation upon administration. Use of releasable linkers allows PEG chains to slowly hydrolyze continuously forming active conjugated IL-2 bound by 2-PEGs or 1-PEG. The location of PEG chains at the IL-2/IL-2Rα interface interferes with binding to high-affinity IL-2Rα, while leaving binding to low-affinity IL-2Rβ unperturbed, favoring immune activation over suppression in the tumor (Charych et al. 2016, Charych et al. 2017).

“IL2v” refers to an IL-2/IL-15Rβγ agonist based on IL-2 by Roche, being an IL-2 variant with abolished binding to the IL-2Rα subunit with the SEQ ID NO: 51. IL2v is used for example in fusion proteins, fused to the C-terminus of an antibody. IL2v was designed by disrupting the binding capability to IL-2Rα through amino acid substitutions F42A, Y45A and L72G (conserved between human, mouse and non-human primates) as well as by abolishing O-glycosylation through amino acid substitution T3A and by avoidance of aggregation by a C125A mutation like in aldesleukin (numbering based on UniProt ID P60568 excluding the signal peptide) (Klein et al. 2017). IL2v is used as a fusion partner with antibodies, e.g. with untargeted IgG (IgG-IL2v) in order to increase its half-life (Bacac et al. 2017). In RG7813 (or cergutuzumab amunaleukin, RO-6895882, CEA-IL2v) IL2v is fused to an antibody targeting carcinoembryonic antigen (CEA) with a heterodimeric Fc devoid of FcγR and C1q binding (Klein 2014, Bacac et al. 2016, Klein et al. 2017). And, in RG7461 (or RO6874281 or FAP-IL2v) IL2v is fused to the tumor specific antibody targeting fibroblast activation protein-alpha (FAP) (Klein 2014).

“THOR-707” refers to an IL-2/IL-15Rβγ agonist based on a site-directed, singly PEGylated form of IL-2 with reduced/lacking IL2Rα chain engagement while retaining binding to the intermediate affinity IL-2Rβγ signaling complex (Joseph et al. 2019) (WO 2019/028419A1).

“ALKS 4230” refers to a circularly permutated (to avoid interaction of the linker with the β and γ receptor chains) IL-2 with the extracellular domain of IL-2Rα selectively targets the By receptor as the α-binding side is already occupied by the IL-2Rα fusion component (Lopes et al. 2020)

“NL-201” refers to IL-2/IL-15Rβγ agonists, which is are computationally designed protein that mimics IL-2 to bind to the IL-2 receptor βγ_{c} heterodimer (IL-2Rβγ_{c}) but has no binding site for IL-2Rα or IL-15Rα (Silva et al. 2019) and WO 2021/081193A1 (NEO 2-15 E62C, SEQ ID NO: 52)).

“NKRT-255” refers to an IL-2/IL-15Rβγ agonist based on a PEG-conjugated human IL-15 that retains binding affinity to the IL-15Rα and exhibits reduced clearance to provide a sustained pharmacodynamic response (WO 2018/213341A1).

“THOR-924, -908, -918” refer to IL-2/IL-15Rβγ agonists based on PEG-conjugated IL-15 with reduced binding to the IL-15Rα with an unnatural amino acid used for site-specific PEGylation (WO 2019/165453A1).

“P-22339” refers to an IL-15/IL-15Rα sushi complex, where IL-15 is bound to the N-terminus of one Fc chain and the IL-15Rα sushi domain is bound to the N-terminus of a second Fc chain as described in WO 2016/095642 and Hu et al. (2018) with the L52C substitution on the IL-15 polypeptide (SEQ ID NO: 64) and the S40C substitution on the IL-15Rα sushi+ polypeptide (SEQ ID NO: 65) forming a disulfide bond.

“XmAb24306” refers to an IL-15/IL-15Rα sushi complex, where a mutant IL-15 is bound to the N-terminus of one Fc chain and the IL-15Rα sushi domain is bound to the N-terminus of a second Fc chain as described in US 2018/0118805 (see XENP024306 in FIG. 94C, SEQ ID NO: 66 and SEQ ID NO: 67).

“ANV419” refers to a fusion protein of IL-2 and an IL-2 specific antibody (as described in Huber et al. poster #571, SITC Annual Meeting 2020, Arenas-Ramirez et al. (2016)).

“XTX202” (CLN-617) refers to an engineered IL-2 prodrug with its activity masked as described in WO 2020/069398 and O'Neil J et al. poster ASCO annual meeting 2021.

“AB248” refers to a fusion protein of an anti-CD8 antibody with an IL-2 as described in Moynihan K et al. “Selective activation of CD8⁺ T cells by a CD8-targeted IL-2 results in enhanced anti-tumor efficacy and safety” poster at SITC 2021.

“WTX-124” refers to a fusion protein of a half-life extension domain, IL-2 and a cleavable inactivation domain as described in Salmeron A. et al., “WTX-124 is an IL-2 ProDrug Conditionally Activated in Tumors and Able to Induce Complete Regressions in Mouse Tumor Models”, poster at AACR annual meeting 2021 and WO 2020/232305A1.

“TransCon IL-2β/γ” refers to an IL-2 with a permanently attached 5 kDa methoxy polyethylene glycol (mPEG) moiety in the IL-2Rα binding site retaining strong IL-2Rβγ activity, and a transiently attached 40 kDa methoxy polyethylene glycol (mPEG) moiety for increasing the in vivo half-life (Rosen et al. 2022) (WO 2021/245130).

“MK-1484” refers to an IL-2 mutein with amino acid substitutions R37A and F41K to selectively bind the IL-2Rβγ and having a non-natural amino acid p-azidomethyl-L-phenylalanineat at or near the N-terminus of the IL-2 polypeptide conjugated to a nonpeptidic, water-soluble polymer (WO 2021/216478A1).

“MDNA11” refers to an IL-2 mutein with C125S substitution with increased affinity to IL-2Rβ and no binding to IL-2Rα fused to human albumin (see SEQ ID NO: 53, WO 2021/258213A1).

“Percentage of identity” between two amino acids sequences means the percentage of identical amino acids, between the two sequences to be compared, obtained with the best alignment of said sequences, this percentage being purely statistical and the differences between these two sequences being randomly spread over the amino acid sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by Smith and Waterman (1981), by using the local homology algorithm developed by Needleman and Wunsch (1970), by using the method of similarities developed by Pearson and Lipman (1988), by using computer software using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar 2004), or by using CLUSTAL (Goujon et al. 2010). To get the best local alignment, one can preferably use the BLAST software with the BLOSUM 62 matrix. The identity percentage between two sequences of amino acids is determined by comparing these two sequences optimally aligned, the amino acids sequences being able to encompass additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

Conservative amino acid substitutions refer to a substation of an amino acid, where an aliphatic amino acid (i.e. Glycine, Alanine, Valine, Leucine, Isoleucine) is substituted by another aliphatic amino acid, a hydroxyl or sulfur/selenium-containing amino acid (i.e. Serine, Cysteine, Selenocysteine, Threonine, Methionine) is substituted by another hydroxyl or sulfur/selenium-containing amino acid, an aromatic amino acid (i.e., Phenylalanine, Tyrosine, Tryptophan) is substituted by another aromatic amino acid, a basic amino acid (i.e. Histidine, Lysine, Arginine) is substituted by another basic amino acid, or an acidic amino acid or its amide (Aspartate, Glutamate, Asparagine, Glutamine) is replaced by another acidic amino acid or its amide.

“In vivo half-life” or T½ refers to the time required for a quantity of a drug to be reduced to half of its initial amount in vivo. The in vivo half-life of a particular drug can be determined in any mammal. For example, the in vivo half-life can be determined in humans, primates or mice. While the in vivo half-life determined in humans may considerably differ from the in vivo half-life in mice, i.e., the in vivo half-life in mice for a certain drug is commonly shorter than the in vivo half-life determined for the same drug in humans, such in vivo half-life determined in mice still gives an indication for a certain in vivo half-life in humans. Hence, from the in vivo half-life determined for a particular drug in mice, the in vivo half-life of the drug in humans can be extrapolated. This is particularly important since the direct determination of the in vivo half-life of a certain drug in humans is rarely possible due to prohibitions of experiments for merely scientific purposes involving humans. Alternatively, the half-life can be determined in primates (e.g., cynomolgus monkeys) which is more similar to the half-life in humans. More specifically, the “in vivo half-life”, (terminal) plasma half-life or T½ is the half-life of elimination or half-life of the terminal phase, i.e. following administration, the in vivo half-life is the time required for plasma/blood concentration to decrease by 50% after pseudo-equilibrium of distribution has been reached (Toutain and Bousquet-Melou 2004). The determination of the drug, here the IL-2/IL-15βγ agonist being a polypeptide, in the blood/plasma is typically done through a polypeptide-specific ELISA.

The term “genetically engineered” means in the context of this invention the artificial manipulation, modification, and recombination of DNA or other nucleic acid molecules in order to modify a cell typically by introducing a vector into the immune cell comprising the respective transgene under control of a constitutive or inducible promoter. Such vector my remain extrachromosomal or may integrate into the genome of the immune cell at a site different to the location of the endogenous gene (if present). Transient genetically engineering may also be achieved through transfection of mRNA encoding the transgene.

The term “treating” as used herein refers to the application or administration of a composition including one or more active agents to a subject, who has cancer, with the purpose to cure, heal, alleviate, relieve, alter, remedy, ameliorate, improve, or affect the disease or the symptoms of the disease.

“An effective amount” as used herein refers to the amount of each active agent required to confer therapeutic effect on the subject, either alone or in combination with one or more other active agents. Effective amounts vary, as recognized by those skilled in the art, depending on route of administration, excipient usage, and co-usage with other active agents.

The compositions of the invention are administered in an effective amount that alone, or together with further doses, produces the desired response, e.g., increases anti-cancer effects (e.g., via increasing viability of the immune cell population disclosed herein and/or enhancing anti-tumor effects of the immune cell population in the tumor microenvironment). In the case of treating a particular disease or condition such as cancer, the desired response includes inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and most preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g., “a”, “an” or “the”, this includes a plural of that noun unless something else is specifically stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”. “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Technical terms are used by their common sense. If a specific meaning is conveyed to certain terms, definitions of terms will be given in the following in the context of which the terms are used.

“qxw”, from Latin quaque/each, every for every x week, e.g., q2w for every second week, q3w for every third week, q4w for every fourth week, and so forth.

“s.c.” for subcutaneously.

“i.v.” for intravenously.

“AUC” for area under the curve.

### DESCRIPTION OF THE INVENTION

It is reported herein, surprisingly, that the combination of an IL-2/IL-15Rβγ agonist leads to enhanced anti-tumor activity of an immune cell population comprising genetically engineered immune cells, more specifically CAR T cells, to express or overexpress a metabolism modulating polypeptide. This was shown in terms of increased proliferation (in vitro) and peripheral expansion (in vivo) with the IL-2/IL-15Rβγ agonist (or in short “agonist). In accordance with the increased proliferation, the T cell phenotype shifted towards the effector memory-like phenotype with enhanced cytotoxic capabilities both in vitro and in vivo. Whereas, IL-2, IL-15 or non-covalent IL-15/IL-15Rα complexes have been used to stimulate immune cell populations with limited success, the co-expression/overexpression of the metabolism modulating polypeptide surprisingly showed a strong increase in the anti-tumor activity of the CAR T cells in vivo.

Accordingly, one embodiment of the present invention is an immune cell population for use in treatment of cancer in a subject, where the immune cell population is administered in combination with an IL-2/IL-15Rβγ agonist, wherein the immune cell population comprises immune cells which are genetically engineered to express or overexpress a polypeptide that (a) improves glucose uptake activity; (b) modulates the Krebs cycle; (c) enhances lactate concentration; or (d) diverts or redirects glucose metabolites out of a glycolysis pathway, referred herein as metabolism modulating polypeptides. Adoptive therapy approaches using autologous or allogeneic transplants of immune cell populations, which are optionally enriched, primed, selected for or modified to express antigen receptors have founded a new era of individualized therapies for cancer patients. Immune cell populations may be derived from peripheral blood mononuclear cells (PBMCs) obtained by leukapheresis from each individual patient (i.e., autologous approach) or from a patient or healthy volunteer (i.e., allogeneic approach). PBMC contain immune cells that are responsive to IL-2/IL-15Rβγ agonists, i.e., cells expressing the IL-2/IL-15Rβγ, which are able to exert strong anti-tumor activity, both through the innate immune response and the adaptive immune response. Accordingly, the immune cell population of this invention comprises immune cells expressing the IL-2/IL-15Rβγ receptor.

Another embodiment of the present invention is an IL-2/IL-15Rβγ agonist for use in treatment or management of cancer in a subject in combination with an immune cell population, wherein the immune cell population comprises immune cells which are genetically engineered to express or overexpress a polypeptide that (a) improves glucose uptake activity; (b) modulates the Krebs cycle; (c) enhances lactate concentration; or (d) diverts or redirects glucose metabolites out of a glycolysis pathway.

The expression or overexpression is mediated by genetic engineering of the immune cell. Typically, this is achieved by introducing a vector or vector genome into the immune cell comprising the respective transgene under control of a constitutive or inducible promoter. Such vector my remain extrachromosomal or may integrate into the genome of the immune cell at a site different to the location of the endogenous gene (if present). Suitable vectors are retroviral or lentiviral vectors that are routinely used also for introducing CARs into the immune cells. Alternatively, adenovirus oder andeno-associated virus vectors may be used. In a preferred embodiment, the conding sequence for such polypeptide is by the same viral vector under in the same expression cassette as the chimeric receptor polypeptide separated, for example, by a P2A ribosomal skip sequence.

“Expressed” is used for metabolism modulating polypeptides, respectively their genes, which are also referred to as bolt-on genes or BOXR genes, introduced into a immune cell which does not express such gene, whereas “overexpressed” is used for metabolism modulating polypeptides, respectively their genes, that are naturally expressed in the immune cell from its endogenous allels, but which introduction of a further copy leads to more of the respective polypeptide in the immune cells, which can be measured by increased RNA expression (e.g., by quantitative PCR), protein expression (e.g., Western Blot) or by measuring the resepective enzyme activity. It is also forseen by the inventors, that more than one metabolism modulating polypeptides is expressed or overexpressed, preferably two metabolism modulating polypeptides.

The expression or overexpression is mediated by genetically engineering of the immune cell. Typically, this is achieved by introducing a vector into the immune cell comprising the respective transgene under control of a constitutive or inducible promoter. Such vector my remain extrachromosomal or may integrate into the genome of the immune cell at a site different to the location of the endogenous gene (if present). Suitable vectors are retroviral or lentiviral vectors that are routinely used also for introducing CARs into the immune cells. Alternatively, adenovirus oder andeno-associated virus vectors may be used. In a preferred embodiment, the conding sequence for such polypeptide is by the same viral vector under in the same expression cassette as the chimeric receptor polypeptide separated, for example, by a P2A ribosomal skip sequence.

In one embodiment, the metabolism modulating polypeptide is present on a vector within the cell or is integrated at a non-endogenous site of the genome within the cell. In one embodiment, the metabolism modulating polypeptide is present in the cell at a copy number that is higher than the endogenous copy number of said metabolism modulating polypeptide. The metabolism modulating polypeptide may be present at a copy number of at least 3 copies, at least 5 copies or at least 7 copies per cell. The copy number refers to the number of genomic copies of the metabolism modulating polypeptide present within a cell.

### Bolt-on Genes-BOXR

It is also reported herein, suprisingly, that immune cells expressing an exogenous copy of a gene modulating the metabolism (e.g., encoding a metabolism modulating (regulating) polypeptide) of the immune cell showed a further enhanced anti-tumor effect when stimulated by an exemplary IL-2/IL-15Rα agonist in vivo.

In some instances, the metabolism modulating polypeptide (a) improves glucose uptake activity; (b) modulates the Krebs cycle; (c) enhances lactate concentration; or (d) diverts or redirects glucose metabolites out of a glycolysis pathway.

### Improving Glucose Uptake

In one embodiment the polypeptide that improves glucose uptake activity is a glucose importation polypeptide. In some examples, the glucose importation polypeptide is a glucose transporter (GLUT) or a sodium-glucose cotransporter (SGLT). Examples include, but are not limited to, GLUT1, GLUT1 S226D variant, GLUT3, GLUT8, GLUT8 L12A L13A variant, GLUT11, GLUT7, GLUT4, SGLT1 or SGLT2. The principle of modulating immune cells by expressing or overexpressing genes improving glucose uptake has been described in WO 2020/010110. The sequences of GLUT1, GLUT1 S226D variant, GLUT3, GLUT8, GLUT8 L12A L13A variant, GLUT11, GLUT7, GLUT4, SGLT1 or SGLT2 are disclosed therein as SEQ ID NOs: 81-90. Further included are functional vairants thereof as dislcosed in WO 2020/010110. The preferred glucose importation polypeptide is GLUT1.

### Modulating the Krebs Cycle

In one embodiment, the polypeptide that modulates the Krebs cycle is:
an enzyme that catalyzes a reaction in the Krebs cycle, for example, isocitrate dehydrogenase (IDH), malate dehydrogenase (MDH), or phosphoglycerate dehydrogenase (PHGDH),
an enzyme that uses a Krebs cycle metabolite as a substrate, for example, glutamic-oxaloacetic transaminase (GOT) or phosphoenolpyruvate carboxykinase 1 (PCK1), or
an enzyme that converts a precursor to a Krebs cycle metabolite, for example, phosphoserine aminotransferase (PSAT1), glutamate dehydrogenase (GDH1), glutamate-pyruvate transaminase 1 (GPT1), or glutaminase (GLS);

The principle of modulating immune cells by modulating the Krebs cylce has been described in WO 2020/037066. The sequences of PCK1, GOT1, GOT2, GPT1, GLS, PSAT1 and GDH1 are disclosed therein as SEQ ID NOs: 86-92. Further included are functional vairants thereof as dislcosed in WO 2020/037066. Preferred Krebs cycle modulating genes are GOT2 and GFPT1.

### Enhancing Lactate Concentration

In one embodiment, the polypeptide that enhances lactate concentration is:
monocarboxylate transporter (MCT), for example, MCT1, MCT2, or MCT4,
an enzyme involved in lactate synthesis, for example, lactate dehydrogenase A (LDHA), or
a polypeptide that inhibits a pathway that competes for lactate-synthesis substrates, for example, pyruvate dehydrogenase kinase 1 (PDK1).

The principle of modulating immune cells by enhancing lactate concentration has been described in WO 2020/051493. The sequences of MCT1, MCT2, MCT4, LDHA and PDK1 are disclosed therein as SEQ ID NOs: 81-85. Further included are functional vairants thereof as dislcosed in WO 2020/051493.

### Diverting or Redirecting Glucose Metabolites Out of a Glycolysis Pathway

In one embodiment, the polypeptide that diverts or redirects glucose metabolites is selected from the group consisting of pyruvate kinase muscle isozyme M2 (PKM2), glutamine-fructose-6-phosphate aminotransferase 1 (GFPT1), and TP53-inducible glycolysis and apoptosis regulator (TIGAR). The principle of diverting or redirecting glucose metabolites out of a glycolysis pathway has been described in WO 2023/049933. The sequences of PKM2, GFPT1 and TIGAR are disclosed therein as SEQ ID NOs: 68-74. Further included are functional vairants thereof as dislcosed in WO 2023/049933. In a preferred embodiment, the polypeptide that diverts or redirects glucose metabolism is TIGAR.

In one embodiment the expression or overexpression of the polypeptide comprising the amino acid sequence of any one of (a)-(d) or derivatives thereof, i.e., a polypeptide that improves glucose uptake activity, modulates the Krebs cycle, enhances lactate concentration, or diverts or redirects glucose metabolites out of a glycolysis pathway, or derivatives thereof lead to an at least 70% increase of proliferation of the immune cell population when compared to the native immune cell population of the same type not expressing or overexpressing such polypeptide. The increase in proliferation of an immune cell population can be determined by the assay described in example 10. The assay may be conducted at different glucose conditions, whereas 1.25 mM glucose reflects more typical energy-deprived conditions in the tumor/tumor microenvironment and 10 mM glucose reflects glucose levels in the peripheral blood. For the low, 1.25 mM, glucose level, preferably the expression or overexpression of the polypeptide lead to an at least 100% increase of proliferation of the immune cell population when compared to the native immune cell population of the same type not expressing or overexpressing such polypeptide.

In a further emobidment, two metabolism modulating polypeptides are transfected into immune cells. The genetically engineered immune cells described herein, co-expressing at least two metabolism modulating polypeptides and a CAR polypeptide can be used in immune therapy such as T-cell therapy (both αβ and γδ T cells), NK-cell therapy or NK T cell therapy for inhibiting and/or killing diseased cells expressing an antigen to which a chimeric antigen receptor (CAR) or T cell receptor (TCR) polypeptide targets, directly or indirectly (e.g., via a therapeutic agent conjugated to a tag to which the CAR polypeptide binds). The at least two metabolism modulating polypeptides are preferably selected from the group consisting of: (a) GOT2 and TIGAR; (b) GOT2 and GLUT1; (c) GOT2 and PDK1; (d) TIGAR and GLUT1; (e) PDK1 and Cystathionine gamma-lyase (CTH), CTH; (f) CTH and Phosphoserine phosphatase (PSPH); (g) GLUT1 and Argininosuccinate synthase 1 (ASS1); and (h) GLUT1 and PSPH. Their co-expression in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the at least two metabolism modulating polypeptides from above and/or a CAR or a TCR polypeptide transiently, to limit any potential non-tumor specific reactivity.

### Vectors

In further embodiments the nucleic acid or nucleic acid set encoding the metabolism modulating polypeptide and/or a chimeric receptor polypeptide is comprised within one or more viral vectors. The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding at least two metabolism modulating polypeptides and/or the chimeric receptor polypeptides. The synthetic linkers may contain nucleic acid sequences that correspond to a particular restriction site in the vector. The selection of expression vectors/plasmids/viral vectors would depend on the type of immune cells for expression of the at least two metabolism modulating polypeptides described herein and/or the chimeric receptor polypeptides, but should be suitable for integration and replication in eukaryotic cells. An exemplary embodiment is a method of modifying the metabolism of immune cells, comprising transfecting immune cells transiently or stably with the vector or vector set and collecting immune cells transfected with the vector or vector set.

Additionally, the vector may contain, for example, some or all of the following: a selectable marker gene, such as the neomycin gene or the kanamycin gene for selection of stable or transient transfectants in immune cells; enhancer/promoter sequences from the immediate early gene of human CMV for high levels of transcription; intron sequences from the human EF1-alpha gene, transcription termination and RNA processing signals from SV40 for mRNA stability; SV40 or polyomavirus origins of replication and ColE1 for proper episomal replication; internal ribosome binding sites (IRESs), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene(s) for assessing expression of the metabolism modulating polypeptide and/or the chimeric receptor polypeptide.

In one specific embodiment, such vectors also include a suicide gene. As used herein, the term “suicide gene” refers to a gene that causes the cell expressing the suicide gene to die. The suicide gene can be a gene (e.g., HSV thymidine kinase) that confers sensitivity to an agent, e.g., a drug (e.g., ganciclovir for HSV thymidine kinase), upon the cell in which the gene is expressed, and causes the cell to die when the cell is contacted with or exposed to the agent. Suicide genes are known in the art (see, for example, Springer, C. J. (Suicide Gene Therapy: Methods and Reviews, Humana Press (2004)) and include, for example, the Herpes Simplex Virus (HSV) thymidine kinase (TK) gene, cytosine deaminase, purine nucleoside phosphorylase, nitroreductase, and caspases such as caspase 8 or caspase-9 (iCasp9). Suitable vectors and methods for producing vectors containing transgenes are well known and available in the art. Examples of the preparation of vectors for expression of metabolism modulating polypeptides and/or chimeric receptor polypeptides can be found, for example, in US 2014/0106449, herein incorporated in its entirety by reference.

Any of the vectors comprising a nucleic acid sequence that encodes the metabolism modulating polypeptides and/or a chimeric receptor polypeptide described herein is also within the scope of the present invention. Such a vector, or the sequence encoding such metabolism modulating polypeptides and/or a chimeric receptor polypeptide contained therein, may be delivered into immune cells such as immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral or gamma-retroviral transduction).

In some embodiments, the vectors for expression of the at least two metabolism modulating polypeptides and/or the chimeric receptor polypeptides are delivered to immune cells by viral transduction (e.g., retroviral transduction such as lentiviral or gamma-retroviral transduction). Exemplary viral methods for delivery include, but are not limited to, recombinant retroviruses (see, e.g., WO 90/07936; WO 94/03622; WO 93/25698; WO 93/25234; WO 93/11230; WO 93/10218; and WO 91/02805; U.S. Pat. Nos. 5,219,740 and 4,777,127; GB 2,200,651; and EP 0345242), alphavirus-based vectors, and adeno-associated virus (AAV) vectors (see, e.g., WO 94/12649, WO 93/03769; WO 93/19191; WO 94/28938; WO 95/11984; and WO 95/00655). In some embodiments, the vectors for expression of the at least two metabolism modulating polypeptides and/or the chimeric receptor polypeptides are retroviruses. In a preferred embodiment, the vectors are lentiviruses.

Examples of references describing retroviral transduction include Anderson et al., U.S. Pat. No. 5,399,346; (Mann et al. 1983); U.S. Pat. Nos. 4,650,764; 4,980,289; (Markowitz et al. 1988); U.S. Pat. No. 5,124,263; WO 95/07358 and (Kuo et al. 1993). WO 95/07358 describes high efficiency transduction of primary B lymphocytes. See also WO 2016/040441A1, all incorporated by reference herein for the purpose and subject matter referenced herein.

In examples in which vectors encoding at least two metabolism modulating polypeptides and/or the chimeric receptor polypeptides are introduced into immune cells, viral vectors or viral particles that capable of infecting the immune cells may be produced by any method known in the art and can be found, for example in WO 91/02805A2, WO 98/09271A1, and U.S. Pat. No. 6,194,191. The viral particles are harvested from the cell culture supernatant and may be isolated and/or purified prior to contacting the viral particles with the immune cells.

In some embodiments, RNA molecules encoding the at least two metabolism modulating polypeptides and/or the chimeric receptor polypeptides as described herein may be prepared by a conventional method (e.g., in vitro transcription) and then introduced into suitable immune cells, e.g., those described herein, via known methods, e.g., Rabinovich, Komarovskaya et al. (2006).

In some instances, the nucleic acid encoding the metabolism modulating polypeptides and/or the nucleic acid encoding a suitable chimeric receptor polypeptide may be cloned into separate expression vectors, which may be introduced into suitable immune cells concurrently or sequentially. For example, an expression vector (or an RNA molecule) for expressing the metabolism modulating polypeptides may be introduced into immune cells first and the transfected immune cells expressing the metabolism modulating polypeptides may be isolated and cultured in vitro. In another example, an expression vector (or an RNA molecule) expressing a suitable chimeric receptor polypeptide can be introduced into the immune cells expressing the metabolism modulating polypeptide(s). In some instances, the nucleic acid(s) encoding the metabolism modulating polypeptides and/or the chimeric receptor polypeptide may be delivered into immune cells via transposons (e.g., piggybac). In some instances, the encoding nucleic acid(s) may be delivered into immune cells via gene editing, for example, by CRISPR, TALEN, zinc-finger nuclease (ZFN), or meganucleases.

In preferred instances, the nucleic acid encoding the metabolism modulating polypeptides and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and the metabolism modulating polypeptides are also within the scope of the present disclosure. Non-limiting examples of useful vectors of the disclosure include viral vectors such as, e.g., retroviral vectors including gamma retroviral vectors and lentiviral vectors, adeno-associated virus vectors (AAV vectors) and adenoviruses.

In some instances, the nucleic acid described herein may comprise two or three coding sequences, one encoding a chimeric receptor polypeptide as described herein, and the other one or two encoding metabolism modulating polypeptides. The nucleic acid comprising the coding sequences described herein may be configured such that the coding sequences encoding the metabolism modulating polypeptides can be expressed as independent (and physically separate) polypeptides. To achieve this goal, the nucleic acid described herein may contain a fourth, optionally fifth or sixth, nucleotide sequence located between the coding sequences for the metabolism modulating polypeptides. This fourth, fifth and/or sixth nucleotide sequence may, for example, encode a ribosomal skip site. A ribosomal skip site is a sequence that impairs normal peptide bond formation. This mechanism results in the translation of additional open reading frames from one messenger RNA. This fourth, fifth and/or sixth nucleotide sequence may, for example, encode a P2A, T2A, or F2A peptide (see, for example, Kim et al. (2011). See Table 1 below.

TABLE 1
Exemplary Ribosomal Skip site

| Ribosomal | | SEQ ID |
|---|---|---|
| Skip Site | Sequence | NO |
| P2A | ATNFSLLKQAGDVEENPGP | 1 |
| T2A | EGRGSLLTCGDVEENP GP | 2 |
| E2A | QCTNYALLKLAGDVESNPGP | 3 |
| F2A | AVKQTLNFDLLKLAGDVESNPGP | 4 |

In another embodiment, the fourth nucleotide sequence may encode an internal ribosome entry site (IRES). An IRES is an RNA element that allows translation initiation in an end-independent manner, also permitting the translation of additional open reading frames from one messenger RNA. Alternatively, the fourth nucleotide sequence may encode a promoter controlling the expression of the second polypeptide and/or the third polypeptide. The fourth nucleotide sequence may also encode more than one ribosomal skipping sequence, IRES sequence, additional promoter sequence, or a combination thereof.

An exemplar IRES sequence is provided below (SEQ ID NO: 5):

GAGGGCCCGGAAACCTGGCCCTGTCTTCTTGACGAGCATTCCTAGG
GGTCTTTCCCCTCTCGCCAAAGGAATGCAAGGTCTGTTGAATGTC
GTGAAGGAAGCAGTTCCTCTGGAAGCTTCTTGAAGACAAACAACG
TCTGTAGCGACCCTTTGCAGGCAGCGGAACCCCCCACCTGGCGAC
AGGTGCCTCTGCGGCCAAAAGCCACGTGTATAAGATACACCTGCA
AAGGCGGCACAACCCCAGTGCCACGTTGTGAGTTGGATAGTIGTG
GAAAGAGTCAAATGGCTCTCCTCAAGCGTATTCAACAAGGGGCTG
AAGGATGCCCAGAAGGTACCCCATTGTATGGGATCTGATCTGGGG
CCTCGGTGCACATGCTTTACATGTGTTTAGTCGAGGTTAAAAAAA
CGTCTAGGCCCCCCGAACCACGGGGACGTGGTTTTCCTTTGAAAA
ACACGATGATAA

The nucleic acid may also include additional coding sequences (including, but not limited to, fifth and sixth coding sequences) encoding further metabolism modulating polypeptides and may be configured such that the polypeptides encoded by the additional coding sequences are expressed as further independent and physically separate polypeptides. To this end, the additional coding sequences may be separated from other coding sequences for a polypeptide by one or more nucleotide sequences encoding one or more ribosomal skipping sequences, IRES sequences, or additional promoter sequences.

In some examples, the nucleic acid (e.g., an expression vector or an RNA molecule as described herein) may comprise coding sequences for at least two metabolism modulation polypeptides and a suitable chimeric receptor polypeptide, the coding sequences (for example, three), in any order, being separated by a fourth nucleotide sequence coding for a P2A peptide. As a result, three separate polypeptides, at least two metabolism modulating polypeptides and the chimeric receptor, can be produced from such a nucleic acid, wherein at least one P2A skip site is linked C-terminally to the upstream polypeptide (encoded by the upstream coding sequence) and the C-terminal residue P from the P2A peptide is linked to the downstream polypeptide (encoded by the downstream coding sequence). In some examples, the chimeric receptor polypeptide is the upstream one and the metabolism modulating polypeptide(s) is/are downstream. In other examples, the metabolism modulating polypeptide(s) is/are upstream and the chimeric receptor polypeptide is downstream.

In some embodiments, the nucleic acid (e.g., an expression vector or an RNA molecule as described herein) may comprise coding sequences of at least two metabolism modulating polypeptides (e.g., those described herein) and a suitable ACTR or CAR polypeptide, the coding sequences, in any order, being separated by a fourth nucleotide sequence coding for a P2A peptide. As a result, two or three separate polypeptides, the metabolism modulating polypeptides and the chimeric receptor polypeptide can be produced from such a nucleic acid, wherein the P2A skip site is linked to the upstream polypeptide (encoded by the upstream coding sequence) and residue P from the P2A peptide is linked to the downstream polypeptide (encoded by the downstream coding sequence).

In some examples, the nucleic acid described above may further encode a linker (e.g., a GSG linker) between two segments of the encoded sequences, for example, between the upstream polypeptide and the P2A peptide.

Alternatively, expression of at least two metabolism modulating polypeptides, and/or a chimeric receptor polypeptide in any of the immune cells disclosed herein can be achieved by introducing RNA molecules. Such RNA molecules can be prepared by in vitro transcription or by chemical synthesis. The RNA molecules can then be introduced into suitable immune cells (e.g., αβ or γδ T or NK cells) by, e.g., electroporation. For example, RNA molecules can be synthesized and introduced into immune cells following the methods described in Rabinovich, Komarovskaya et al. (2006) and WO 2013/040557.

In certain embodiments, a vector(s) or RNA molecule(s) comprising at least two metabolism modulating polypeptides and/or the chimeric receptor polypeptide may be introduced to the immune cells in vivo. As a non-limiting example, this may be accomplished by administering a vector or RNA molecule described herein directly to the subject (e.g., through intravenous administration).

Therefore, in a preferred embodiment, the immune cell population comprises immune cells selected from B cells, αβ T cells, γδ T cells, natural killer (NK) cells or NKT cells. Recent results of clinical trials with infusions of chimeric receptor-expressing autologous T lymphocytes (αβ T cells) provided compelling evidence of their clinical potential. (Brentjens et al. 2003, Pule et al. 2008, Brentjens et al. 2011, Porter et al. 2011, Kochenderfer et al. 2012, Till et al. 2012, Brentjens et al. 2013). Initially, the field was focusing on αβ T cells; accordingly, in a preferred embodiment the immune cells are αβ T cells.

More recently, cell-based therapy has expanded to include natural killer (NK) cells due to their unique advantages such as low risk of on-target/off-tumor toxicity in normal tissues, cytokine release syndrome and neurotoxicity. They also exhibit natural cytotoxicity against tumor cells. Finally, due to reduced graft versus host disease (GVHD), an off-the-shelf cell therapy product (i.e., allogeneic) may be prepared (Schmidt et al. 2020, Wang et al. 2020, Xie et al. 2020, Gong et al. 2021, Wrona et al. 2021). Therefore, NK cells are preferred immune cells.

Another subtype of T cells, i.e., γδ T cells, has immense potential in cell therapy sharing similar advantages of that of NK cells additionally displaying immune regulatory functions (Sievers et al. 2020, Park and Lee 2021), which makes them another preferred embodiment.

In addition, persistent activation of these αβ T cells due to antigen dependent or independent CAR activation can lead to exhaustion and reduced bioactivity, which is again an advantage of the innate cells such as NK and γδ T cells. Finally, both NK and γδ T cells expressing a chimeric receptor polypeptide may have increased effector functions such as increased inflammatory cytokine production, antigen acquisition and presentation or ability to activate adaptive immune responses.

For example, in the recent years NK cells received a growing interest in cancer therapy. NK cells suitable for therapy may be haploidentical NK cells, umbilical cord blood NK cells, stem cell-derived NK cells, NK cell lines, adaptive NK cells, or cytokine-induced memory-like NK cells. They also may be genetically modified to express a chimeric antigen receptor (CAR). Cytokines or cytokine-based molecules including N-803 (ALT-803) are being explored to augment the cytotoxicity and longevity of NK cells, but also NK cell-engager molecules and immune-checkpoint inhibitors. (Du et al. 2021, Myers and Miller 2021). Similarly, γδ T cells gain increasing interest in cancer therapy, both in a genetically unmodified or genetically engineered version expressing CARs or engineered TCR (Nussbaumer and Koslowski 2019). Further, tumor-infiltrating lymphocytes (TILs) obtained from infiltrated lymphnodes and/or derived from tumor tissue are considered a treatement option in cancer therapy. For example, tumor tissues are digested into small fragments or a single cell suspension and then expanded in culture with IL-2. Reactive TILs may then be enriched/selected by their recognition of autologous tumor cells. In a different approach, the “young TIL” approach, the selection stop is ommited. The TIL culture is finally expanded to a clinically relevant level and infused back into the patient. (Wang et al. 2021). Recently, with lifileucel a first TIL-based therapy has been approved by FDA for the treatment of patients with unresectable or metastatic melanoma. Clinically, most succesful are presently the genetically modified T cell therapies, especially T cells genetically engineered to express a CAR (Sterner and Sterner 2021) and optionally further transgenes as a safety switch, a cytokine, a metabolism modulating transgene etc.,

In one embodiment, the immune cells comprise one or more of the following features:
(I) the immune cells are autologous;
(II) the immune cells are allogeneic;
(III) the immune cells are activated, expanded, or both ex vivo, and
(IV) the immune cells are activated ex vivo in the presence of one or more of 4-1BB ligand, anti-4-1BB antibody, anti-CD3 antibody, anti-CD28 antibody, IL-15, anti-IL-15 receptor antibody, IL-2, IL-7, IL-12, IL-21 and K562 cells, and an engineered artificial stimulatory cell or particle prior to administration.

In any of the compositions or methods described herein, the immune cells may be autologous to the subject or patient, i.e., the immune cells are obtained from the subject in need of the treatment, optionally genetically engineered, and then administered to the same subject. In one specific embodiment, prior to re-introduction into the subject, the autologous immune cells are activated and/or expanded ex vivo. Administration of autologous cells to a subject may result in reduced rejection of the immune cells as compared to administration of non-autologous cells.

Alternatively, the immune cells are allogeneic cells, i.e., the cells are obtained from a first subject, optionally genetically engineered, and administered to a second subject that is different from the first subject but of the same species. For example, allogeneic immune cells may be derived from a human donor and administered to a human recipient who is different from the donor. NK cells and γδ T cells are preferably used to generate off-the-shelf cell therapies. In a specific embodiment, T cells are allogeneic T cells in which the expression of the endogenous T cell receptor has been inhibited or eliminated. In one specific embodiment, prior to introduction into the subject, the allogeneic immune cells are activated and/or expanded ex vivo.

NK and T cells (αβ T or γδ T cells) can be activated by any method known in the art, e.g., in the presence of one or more agents selected from the group consisting of 4-1BB ligand, anti-4-1BB antibody, anti-CD3 antibody, anti-CD28 antibody, IL-15, anti-IL-15 receptor antibody, IL-2, IL-7, IL-12, IL-21 and K562 cells, and/or engineered artificial stimulatory cells or particles. See, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CD137L). Such cell lines include, but are not necessarily limited to, K562 [ATCC, CCL 243; (Lozzio and Lozzio 1975, Klein et al. 1976)], and the Wilms tumor cell line HFWT (Fehniger and Caligiuri 2001, Harada et al. 2004), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 (Harada et al. 2002).

In some instances, the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface. T lymphocytes can be activated by any method known in the art, e.g., in the presence of anti-CD3/CD28, IL-2, IL-15, phytohemoagglutinin, engineered artificial stimulatory cells or particles, or a combination thereof. In additional aspects, the method further comprises cryopreserving the population of immune cells for storage and/or transport to clinical sites.

The immune cell may be derived from one or more cell lines, from peripheral blood mononuclear cells (PBMC), hematopoietic stem cells (HSCs), cord blood stem cells or induced pluripotent stem cells (iPSCs); or from tumor tissue.

In some other embodiments, the immune cells described herein can be derived from a cell line, e.g., selected from NK-92, NK-92 MI, YTS, and KHYG-1, preferably NK-92 cells. In other embodiments, the immune cells described herein can be derived from peripheral blood mononuclear cells (PBMC), hematopoietic stem cells (HSCs), cord blood stem cells (CBSCs) or induced pluripotent stem cells (iPSCs). In other embodiments, the immune cell population described herein can be obtained from other sources, such as bone marrow, or tissues such as spleen, lymph node, thymus, or tumor tissue. In some instances, the starting population of NK or T cells is obtained from isolating mononuclear cells using ficoll-paque density gradient. In some instances, the method further comprises depleting the mononuclear cells of CD3, CD14, and/or CD19 cells to obtain the starting population of NK cells. In a particular instance, depleting comprises performing magnetic sorting. In some instances, NK cells could be positively selected using sorting, magnetic bead selection or other methods to obtain the starting populations of NK cells.

In one embodiment, specifically lymphocytes are obtained from tumor tissue, i.e., TILs. A source suitable for obtaining the type of immune cells desired would be evident to one of skill in the art. In some examples, the immune cells can be a mixture of different types of T cells and/or NK cells as known in the art. For example, the immune cells can be a population of immune cells isolated from a suitable donor (e.g., a healthy volunteer for allogeneic approaches or patient). In a preferred embodiment, the population of immune cells is derived from PBMCs, which may be obtained from the patient, who is in need for the treatment described herein (autologous approach). The type of immune cells desired (e.g., T cells or NK cells) may be expanded within the population of cells obtained by co-incubating the cells with stimulatory molecules as described supra.

Additionally, immune cells such as NK cells are derived from cord blood stem cells or induced pluripotent stem cells (iPSCs) providing from “off-the shelf” source for immunotherapy (Li et al. 2018, Liu et al. 2018, Morgan et al. 2020, Wrona et al. 2021). In particular embodiments, the starting population of NK cells is obtained from cord blood. In other embodiments, the cord blood has previously been frozen. In some embodiments, cells are derived from cell lines (e.g., NK-92 and Vy9V82 T cell).

In a preferred embodiment, the immune cells derived from tumor tissue are TILs. Methods for obtaining are known in the art. (Wang et al. 2021).

### Chimeric Receptor Polypeptides

In one embodiment of the invention, the immune cell population comprises immune cells, which are genetically engineered to express a chimeric receptor polypeptide, wherein the chimeric receptor polypeptide comprises an extracellular target binding domain, a transmembrane domain; and a cytoplasmic signaling domain. As used herein, a chimeric receptor polypeptide refers to a non-naturally occurring molecule that can be expressed on the surface of an immune cell and comprises an extracellular target binding domain, a transmembrane domain and at least one cytoplasmic signaling domain. The extracellular target binding domain targets an antigen of interest (e.g., an antigen associated with a disease such as cancer). It may bind to an antigen of interest directly (e.g., an extracellular antigen binding domain in a CAR polypeptide as disclosed herein), or may bind to the antigen of interest via an intermediate, for example, an Fc-containing agent such as an antibody. The transmembrane domain ankers the chimeric receptor polypeptide within the cellular membrane of an immune cell. The chimeric receptor polypeptides are configured such that, when expressed in an immune cell, the extracellular target binding domain is located extracellularly for binding to a target antigen, directly or indirectly, whereas the cytoplasmic signaling domain is located intracellularly to allow for signaling into the cell upon binding of the target binding domain to the target. A chimeric receptor polypeptide may further comprise a hinge domain, one or more co-stimulatory domains, or a combination thereof.

### Chimeric Antigen Receptor (CAR)

In one embodiment, the chimeric receptor polypeptide is a chimeric antigen receptor (CAR) polypeptide. As used herein, a CAR polypeptide (a.k.a., a CAR construct) refers to a non-naturally occurring molecule that can be expressed on the surface of an immune cell and comprises an extracellular antigen binding domain, together with the transmembrane domain and the cytoplasmic signaling domain. The CAR polypeptides described herein may further include at least one co-stimulatory signaling domain. The extracellular antigen binding domain re-directs the specificity of immune cells (e.g., NK, αβ T or γδ T cells) expressing the CAR polypeptide to the target tumor cells. As used herein, “an extracellular antigen binding domain” refers to a peptide or polypeptide having binding specificity to a target antigen of interest, which can be a naturally occurring antigen. Such a target antigen may be any molecule that is associated with a disease or condition, including, but are not limited to, tumor antigens. In some embodiments, the target antigen binding domain targets a native tumor antigen protein. In other embodiments, the target antigen binding domain targets a variant (e.g., mutation) of a tumor antigen protein. Some examples include EGFRvIII scFv recognizes the tumor specific variant of EGFR (Wang et al. 2020).

As also cells infected by pathogens can be targeted by immune cells using CARs or TCRs, in one embodiment of the invention the target antigens are pathogenic antigens, e.g., bacterial, fungal, or viral antigens, or antigens present on diseased cells, such as those described herein. Accordingly, the immune cell population of this invention is used in one embodiment for use in the treatment of infectious diseases caused by bacteria, fungi or viruses.

Non-limiting examples of the extracellular antigen binding domains are tumor antigens, pathogenic antigens and immune cells specific to an autoantigen (Gubin et al. 2015, Linnemann et al. 2015). Respective diseases and/or conditions to be treated include tumors, inflammatory conditions and auto-immune disorders. In some embodiments, the antigen is selectively expressed or overexpressed on cells of the disease or condition, e.g., the tumor or pathogenic cells, as compared to normal or non-targeted cells or tissues. In some embodiments the extracellular antigen binding domain binds to a tumor antigen, which is associated with a hematologic or solid tumor.

Non-limiting examples of hematologic tumor extracellular binding domains are domains of CD19, CD20, CD22, Kappa-chain, CD30, CD123, CD33, LeY, CD138, CD5, BCMA, CD7, CD40, ROR1 and IL-1RAP. Non-limiting examples of solid tumor extracellular binding domains are domains of GD2, GPC3, FOLR (e.g., FOLR1 or FOLR2), HER2, EphA2, EGFRVIII, IL13RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, L1-CAM, B7H3, GUCY2C, Nectin4, LRRC15, PSMA and CAIX. In other instances, tumor antigens are derived from cancers that are characterized by tumor-associated antigen expression, such as HER2 expression. Antigens may include epitopic regions or epitopic peptides derived from genes mutated in tumor cells or from genes transcribed at different levels in tumor cells compared to normal cells (e.g., survivin, mutated Ras, bcr/abl rearrangement, HER2, mutated or wild-type p53).

The extracellular antigen binding domain as described herein does not comprise the Fc portion of an immunoglobulin, and may not bind to an extracellular domain of an Fc receptor (FcR). An extracellular domain that does not bind to an FcR means that the binding activity between the two is not detectable using a conventional assay or only background or biologically insignificant binding activity is detected using the conventional assay.

In some instances, the extracellular antigen binding domain of any CAR polypeptides described herein is a peptide or polypeptide capable of binding to a cell surface antigen (e.g., a native and mutated tumor antigen), and may be presented on the cell surface of an antigen-presenting cell.

In some embodiments, the extracellular antigen binding domain is a single-chain variable fragment (scFv). In some embodiments, extracellular antigen binding domain is a single domain antibody. In exemplary embodiments, the scFv or single domain antibody binds to a tumor antigen, a pathogenic antigen, or an immune cell specific to an autoantigen. These may be derived from an antibody that binds the target cell surface antigen with a high binding affinity. Table 2 lists exemplary cell-surface target antigens and exemplary antibodies binding to such.

TABLE 2
Exemplary Cell Surface Target Antigen and Exemplary Antibodies Binding to Such

| | | | Exemplary |
|---|---|---|---|
| Exemplary target | Exemplary | Exemplary target | antibodies and Fc- |
| antigens | antibodies | antigens | fusion agents |
| CD137 (4-1BB) | utomilumab | CD74 | milatuzumab |
| Trophoblast | naptumomab | HLA-DR | IMMU-114 |
| glycoprotein (5T4) | estafenatox | | |
| Adenosine A2a | anti-A2aR mAbs | Hsp70 | mi-TUMEXtx |
| receptor (A2aR) | | | |
| Alk-1 protein kinase | ascrinvacumab | Hsp 90 | ZSG-102 |
| (ACVRL1) | | | |
| ADAM-10 | 8C7 | ICAM-1 | BI-505 |
| (ADAM10) | | | |
| TACE (ADAM17) | MEDI-3622 | Inducible T-cell co- | GSK-3359609 |
| | | stimulator (ICOS) | |
| ADAM-28 | GFC-201 | Immunoglobulin κ | KappaMab |
| (ADAM28) | | (Ig κ) | |
| CD156; | MAB-1031 | Immunoglobulin λ | LambdaMab |
| Immunoglobulin G1; | | (Ig λ) | |
| Immunoglobulin G2 | | | |
| (ADAM8) | | | |
| ADAM-9 (ADAM9) | AEX-6003 | IL-6 receptor (IL-6R) | tocilizumab |
| Anterior gradient | agtuzumab | IL-7 receptor (IL-7R) | anti-IL7R mAbs |
| protein 2 homolog | | | |
| (AGR2) | | | |
| Anaplastic | KTN-0125 | IL-13 receptor alpha 1 | ASLAN-004 |
| lymphoma kinase | | subunit (IL13RA1) | |
| (ALK) | | | |
| Angiopoietin ligand- | vanucizumab | IL-13 receptor alpha 2 | anti-IL13RA2 mAbs |
| 2 (Ang-2); Vascular | | subunit (IL13RA2) | |
| endothelial growth | | | |
| factor-A (VEGF-A) | | | |
| Lactadherin (Anti- | TriAb (11D10) | IL-1 receptor | CAN-04 |
| idiotype) | | accessory protein | |
| | | (IL1RAP) | |
| Tumor necrosis | BION-1301 | IL-2 receptor beta | Mikbeta1 |
| factor ligand 13 | | (IL2R beta) | |
| (APRIL) | | | |
| Aspartate beta- | PAN-622 | Immunoglobulin like | BAY-1905254 |
| hydroxylase (ASPH) | | domain receptor 2 | |
| | | (ILDR2) | |
| Axl tyrosine kinase | BA-3011 | Integrin alpha-X/beta- | anti-Integrin a10b1 |
| (AXL) | | 1 (Integrin a10b1) | mAbs |
| CD276 antigen (B7- | BVD m276; hu8H9 | Integrin alpha-3/beta- | BCMab-1 |
| H3 | | 1 (Integrin a3b1) | |
| V-set domain- | FPA-150 | Integrin alpha-6/beta- | 90Y-ITGA6B4 |
| containing T-cell | | 4 (Integrin a6b4) | |
| activation inhibitor 1 | | | |
| (VTCN1; also B7- | | | |
| H4) | | | |
| B-cell activating | blisibimod | Integrin alpha-9 | GND-001 |
| factor; (BAFF; also | | (Integrin a9) | |
| TNFSF13B and | | | |
| CD257) | | | |
| B-cell activating | VAY736 | CD49b (Integrin alpha | Vatelizumab |
| factor receptor; | | 2) | |
| (BAFF-R; also | | | |
| TNFSF13C and | | | |
| CD268) | | | |
| BAG molecular | anti-BAG3 mAbs | CD49c (Integrin alpha | anti-CD49c mAbs |
| chaperone regulator | | 3) | |
| 3 (BAG3) | | | |
| Basigin (BSG; | cHAb18 | CD49d; (Integrin | anti-CD49d mAbs |
| CD147) | | alpha 4) | |
| B-cell maturation | SEA-BCMA | CD51 | abituzumab |
| antigen (BCMA; | | | |
| also TNFRSF17) | | | |
| ADP ribosyl | OX-001 | CD29 (integrin beta | OS-2966 |
| cyclase-2 (BST1) | | 1) | |
| B and T lymphocyte | 40E4 | CD61 (Integrin beta | anti-CD61 mAbs |
| attenuator (BTLA) | | 3) | |
| Complement C5a | neutrazumab | Jagged-1 | anti-Jagged-1 mAbs |
| receptor (C5aR) | | | |
| CACNA2D1 | anti-CACNA2D1 | Kidney-associated | AB-3A4 |
| calcium channel | mAbs | antigen 1 (KAAG1) | |
| subunit | | | |
| (CACNA2D1) | | | |
| Carbonic anhydrase- | G250 | Potassium channel | Y-4 |
| IX (CAIX) | | subfamily K member | |
| | | 9 (KCNK9) | |
| Calreticulin (CALR) | Anti-CALR mAbs | KIR2DL1/2L3 | lirilumab |
| Caveolin 1 (CAV1) | anti-CAV1 mAbs | tyrosine-protein | CDX-0158 |
| | | kinase kit (KIT) | |
| Carbonic anhydrase- | 177Lu-6A10-Fab; | L1CAM | anti-L1CAM mAbs |
| XII (CAXII) | anti-CAXII mAbs | | |
| CCR2 chemokine | plozalizumab | Death receptor 5 | APOMAB |
| receptor (CCR2) | | (DR5) | |
| CCR3 chemokine | anti-CCR3 mAbs | CD223 (LAG3) | relatlimab |
| receptor (CCR3) | | | |
| CCR4 chemokine | mogamulizumab | Lewis Y | hu3S193; MB311 |
| receptor (CCR4) | | | |
| CCR5 chemokine | PRO 140; | Zinc transporter | SGN-LIV1 |
| receptor (CCR5) | CCR5mAb004 | SLC39A6 (LIV1) | |
| CCR7 chemokine | anti-CCR7 mAbs | Lysyl oxidase-like | AB-0023 |
| receptor (CCR7) | | protein 2 (LOXL2) | |
| CCR9 chemokine | anti-CCR9 mAbs | Leucine rich repeat- | ABBV-085 |
| receptor (CCR9) | | containing protein 15 | |
| | | (LRRC15) | |
| Interleukin-3 | CSL362; KHK2823 | Leucine rich repeat- | ARGX-115 |
| receptor alpha | | containing protein 32 | |
| (IL3RA; CD123) | | (LRRC32) | |
| Aminopeptidase N | MI-130110 | Lymphocyte antigen | MEN-1309 |
| (CD13) | | 75 (LY75) | |
| Prominin 1 (CD133) | anti-CD133 mAbs | Ly6/PLAUR domain- | BAY-1129980 |
| | | containing protein 3 | |
| | | (LYPD3) | |
| Syndecan-1 (CD138) | indatuximab | Melanoma associated | LxC-002 |
| | ravtansine | antigen (MAGE | |
| | | peptide presented in | |
| | | MHC) | |
| CD160 | ELB-021 | Matriptase (ST14) | anti-ST14 mAbs |
| Activated leukocyte | CX-2009 | MICA/B | IPH4301 |
| cell adhesion | | | |
| molecule (CD166) | | | |
| B-lymphocyte | MOR208 | MIF/HLA-A2 (MIF | RL21A |
| antigen CD19 | | peptide presented in | |
| | | MHC) | |
| B-lymphocyte | rituximab; | Anti-mullerian | GM-102 |
| antigen CD20 | obinituzumab; | hormone II (MHR2) | |
| | ocaratuzumab | | |
| Membrane glycol- | samalizumab | MMP1/HLA (MMP1 | Anti-MMP1/HLA |
| protein OX2 CD200 | | peptide presented in | mAbs |
| | | MHC1) | |
| CD22 | epratuzumab | Metalloprotease-9 | andecaliximab |
| | | (MMP9) | |
| Immunoglobulin | lumiliximab | Mesothelin (MSLN) | MORAb-009 |
| epsilon Fc receptor | | | |
| II (CD23) | | | |
| Signal transducer | anti-CD24 mAbs | Mucin 1 (MUC1) | PankoMab-GEX |
| CD24 | | | |
| IL-2 receptor alpha | 90Y-daclizumab | Mucin 13 (MUC13) | anti-MUC13 mAbs |
| subunit CD25 | | | |
| CD27 | varilumab | Endomucin (MUC14) | anti-MUC14 mAbs |
| CD28 | theralizumab | Mucin 16 (MUC16) | sofituzumab |
| CD3 | Muromonab-CD3 | Cell surface glyco- | AA98 |
| | (OKT3) | protein MUC18 | |
| | | (CD146) | |
| CD30 | brentuximab vedotin | Mucin 5AC | ensituximab |
| | | (MUC5AC) | |
| Immunoglobulin | BI-1206 | N-glycolyl GM3 | 99mTc-labeled 14F7 |
| gamma Fc receptor | | (NeuGcGM3) | |
| IIB (CD32B) | | | |
| CD33 | lintuzumab | Sodium-dependent | XMT-1536 |
| | | phosphate transport | |
| | | protein 2B | |
| | | (SLC34A2) | |
| CD37 | otlertuzumab | Nucleolin (NCL) | anti-nucleolin mAbs |
| ADP ribosyl | daratumumab | Nectin-4 | enfortumab vedotin |
| cyclase-1 (CD38) | | | |
| CD39 | OREG-103 | Neurofibromin (NF1) | anti-neurofibromin |
| | | | mAbs |
| CD4 | IT-1208 | NGcGM3 ganglioside | racotumomab |
| CD40 | lucatumumab | NKG2A | monalizumab |
| CD43 | leukotuximab | non-POU domain- | PAT-LM1 |
| | | containing octamer- | |
| | | binding protein | |
| | | (NONO) | |
| CD44 | RG7356 | Notch-1 | brontictuzumab |
| CD45 | 131I-BC8 | CD73 | oleclumab |
| Membrane cofactor | AugmAb | Netrin-1 (NTN1) | NP-137 |
| protein (CD46) | | | |
| CD47 | Hu5F9-G4 | OX-40 | PF-04518600 |
| CD52 | alemtuzumab | P2X purinoceptor 7 | BIL-010t |
| | | (P2RX7) | |
| CD55 | PAT-SC1 | FGF receptor (pan | MM-161 |
| | | FGFR) | |
| Neural cell adhesion | IMGN-901 | Integrin (Pan integrin) | NOD201 |
| molecule 1; (CD56) | | | |
| T-cell differentiation | itolizumab | P-cadherin, also | PCA-062 |
| antigen CD6 | | cadherin-3 (CDH3) | |
| CD70 | SGN-70 | Programmed cell | pembrolizumab |
| | | death protein 1 (PD-1) | |
| CD79b | polatuzumab vedotin | Programmed cell | avelumab; Euchloe |
| | | death ligand 1 (PD- | H12 |
| | | L1) | |
| CD8 | anti-CD8 mAbs | Programmed cell | rHIgM12B7 |
| | | death ligand 2 (PD- | |
| | | L2) | |
| CD80 | galiximab | PDGF receptor alpha | olaratumumab |
| | | (PDGFRA) | |
| CD98 | IGN-523 | Placenta specific | anti-PLAC1 mAbs |
| | | protein 1 (PLAC1) | |
| CD99 | NV-103 | PR1/HLA (PR1 | anti-PR1/HLA mAbs |
| | | peptide in MHC) | |
| Cadherin-1 (CDH1) | anti-CDH1 mAbs | Prolactin receptor | ABBV-176 |
| | | PRLR | |
| Cadherin-17 | anti-CDH17 mAbs | Phosphatidylserine | anti- |
| (CDH17) | | | phosphatidylserine |
| | | | mAbs |
| Cadherin 19 | anti-CDH19 mAbs | Prostate stem cell | anti-PSCA mAbs |
| (CDH19) | | antigen (PSCA) | |
| Cadherin-6 (CDH6) | HKT-288 | Glutamate | ATL-101 |
| | | carboxypeptidase II | |
| | | (PSMA) | |
| CD66a (CEACAM1) | CM-24 | Parathyroid hormone- | CAL |
| | | related protein (PTH- | |
| | | rP) | |
| CD66e (CEACAM5) | IMMU-130 | Tyrosine-protein | cofetuzumab |
| | | kinase-like 7 (PTK7) | pelidotin |
| CD66c; CD66e | NEO-201 | Protein tyrosine | PRL3-zumab |
| (CEACAM5/6) | | phosphatase IVA3 | |
| | | (PTP4A3) | |
| Claudin-18.2 | IMAB362 | Poliovirus receptor | COM-701 |
| (CLDN 18.2) | | related immunoglo- | |
| | | bulin domain | |
| | | containing (PVRIG) | |
| Claudin 6 | IMAB027 | Receptor activator of | denosumab |
| | | nuclear factor kappa- | |
| | | B ligand (RANKL) | |
| SLAM family | elotuzumab | Recepteur d'origine | anti-RON mAbs |
| member 7 (CS1) | | nantais (RON) | |
| colony stimulating | cabiralizumab | Tyrosine-protein | cirmtuzumab |
| factor-1 receptor | | kinase transmembrane | |
| (CSF1R) | | receptor ROR1 | |
| | | (ROR1); also | |
| | | NTRKR1 | |
| Cytotoxic T- | ipilumumab | Tyrosine-protein | BA-3021 |
| lymphocyte protein- | | kinase transmembrane | |
| 4 (CTLA4) | | receptor ROR2 | |
| | | (ROR2); also | |
| | | NTRKR2 | |
| Coxsackievirus and | anti-CXADR mAbs | R-spondin-3 (RSPO3) | rosmantuzumab |
| adenovirus receptor | | | |
| (CXADR) | | | |
| CXCR2 chemokine | anti-CXCR2 mAbs | Sphingosine-1- | EDD7H9 |
| receptor | | phosphate receptor 3 | |
| | | (S1PR3) | |
| CXCR3 chemokine | anti-CXCR3 mAbs | Surface Antigen In | IGN-786 |
| receptor | | Leukemia (SAIL) | |
| CXCR4 chemokine | ulocuplumab | Semaphorin-4D | VX-15 |
| receptor | | (SEMA4D) | |
| CXCR5 chemokine | STI-B030X | carbohydrate antigen | MVT-1075 |
| receptor | | 19-9 (CA 19-9) | |
| CXCR7 chemokine | anti-CXCR7 mAbs | Sialyl Thomsen | anti-STn mAbs |
| receptor | | nouveau antigen | |
| | | (STn) | |
| DCLK1 | anti-DCLK1 mAbs | Sialic acid-binding Ig- | AK-002 |
| | | like lectin 8 (Siglec-8) | |
| Dickkopf-related | BHQ-880 | Sialic acid-binding Ig- | anti-Siglec-9 mAbs |
| protein 1 (DKK1) | | like lectin 9 (Siglec-9) | |
| DLK1 | ADCT-701 | Signal Regulatory | OSE-172 |
| | | Protein Alpha | |
| | | (SIRPA) | |
| Delta-like protein | SC16LD6.5 | CD48; also SLAM | SGN-CD48A |
| ligand 3 (DLL3) | | family member 2 | |
| | | (SLAMF2) | |
| Delta-like protein | navicixizumab | CD352; SLAM family | SGN-CD352A |
| ligand 4 (DLL4); | | member 6 (SLAMF6) | |
| VEGF (VEGF) | | | |
| Dipeptidyl | YSCMA | Neutral amino acid | KM-8094 |
| peptidase-4 (DPP4), | | transporter B0 | |
| (also CD26) | | (SLC1A5) | |
| Death receptor-3 | PTX-35 | Somatostatin 2 | XmAb-18087 |
| (DR3) | | receptor (SSTR2) | |
| TRAIL-1 receptor | HuYON007 | Stabilin 1 (STAB1) | FP-1305 |
| (DR4) | MultYbody | | |
| TRAIL-1 receptor; | DR4/DR5 Surrobody | Metalloreductase | 89Zr-DFO- |
| TRAIL-2 receptor | | (STEAP1) | MSTP2109A |
| (DR4/DR5) | | | |
| TRAIL-2 receptor | DS-8273 | Survivin | anti-survivin mAbs |
| (DR5) | | | |
| EGF-like protein 6 | anti-EGFL6 mAbs | TAG-72 | 90Y-IDEC-159 |
| (EGFL6) | | | |
| Epidermal growth | cetuximab; Sym004; | T cell receptor (TCR) | anti-TCR mAbs |
| factor receptor | nimotuzumab | | |
| (EGFR) | | | |
| Epidermal growth | ABT-806 | Endosialin (TEM1) | ontuxizumab |
| factor receptor vIII | | | |
| (EGFRvIII) | | | |
| Epithelial membrane | ONCR-201 | Anthrax toxin | anti-TEM8 mAbs |
| protein 2 (EMP2) | | receptor 1 | |
| | | (ANTXR1); also | |
| | | TEM8 | |
| Endoglin | carotuximab | Tissue factor (TF) | MORAb-066 |
| Ectonucleotide | AGS-16C3F | Transforming growth | anti-TGFBR2 mAbs |
| pyrophosphatase/ | | factor, beta receptor II | |
| phosphodiesterase | | TGF-beta type II | |
| family member 3 | | (TGFBR2) | |
| (ENPP3) | | | |
| Prostaglandin E₂ | anti-PTGER2 mAbs | Thomsen- | JAA-F11 |
| receptor 2 | | Friedenreich Antigen | |
| (PTGER2) | | | |
| Prostaglandin E₂ | anti-PTGER4 mAbs | T cell | BMS-986207 |
| receptor 4 | | immunoreceptor with | |
| (PTGER4) | | Ig and ITIM domains | |
| | | (TIGIT) | |
| EpCAM | oportuzumab | Hepatitis A virus | CDX-014 |
| | monatox | cellular receptor 1 | |
| | | (HAVCR1); also | |
| | | TIM-1 | |
| Ephrin type-A | MEDI-547 | Hepatitis A virus | MBG453 |
| receptor 2 (EphA2) | | cellular receptor 2 | |
| | | (HAVCR2); also | |
| | | TIM-3 | |
| Ephrin type-A | KB004 | Toll-like receptor 2 | OPN-305 |
| receptor 3 (EphA3) | | (TLR-2) | |
| Fibroblast activation | F19 | Toll-like receptor 4 | anti-TLR4 mAbs |
| protein (FAP) | | (TLR-4) | |
| CD95 (FAS) | asunercept | Transmembrane 4 L6 | anti-TM4SF1 mAbs |
| | | family member 1 | |
| | | (TM4SF1) | |
| Fc receptor like | RG-6160 | Tumor necrosis factor | anti-TNFR2 mAbs |
| protein 5 (FCRL5) | | receptor 2 (TNFR2) | |
| FGF receptor 1 | FP-1039 | CD71 | anti-CD71 mAbs |
| (FGFR1) | | | |
| FGF receptor 2b | FPA-144 | Triggering receptor | anti-TREM1 mAbs |
| (FGFR2b) | | expressed on myeloid | |
| | | cells 1 (TREM1) | |
| FGF receptor 3 | B-701 | Tumor-associated | DS-1062 |
| (FGFR3) | | calcium signal | |
| | | transducer 2 (Trop-2) | |
| fms-like tyrosine | Flysyn | TWEAK Receptor | MRT-101 |
| kinase 3 (FLT3) | | (TWEAKR) | |
| Folate receptor alpha | farletuzumab; | Tyrosine-protein | ELB-031 |
| (FOLR1) | IMGN853; | kinase receptor | |
| | KHK2805 | TYRO3 (TYRO3) | |
| Folate receptor beta | anti-FOLR beta | Urokinase receptor | MNPR-101 |
| (FOLR2) | mAbs | (uPAR) | |
| Frizzled-1; Frizzled- | vantictumab | VEGF-2 (VEGFR2) | ramucirumab |
| 2; Frizzled-5; | | | |
| Frizzled-7; Frizzled- | | | |
| 8; (FZD1, 2, 5, 7, 8) | | | |
| Follistatin-like | anti-FSTL1 mAbs | Vimentin | pritumumab |
| protein 1 (FSTL1) | | | |
| Fucosyl-GM1 | BMS-986012 | V-domain Ig | JNJ-61610588 |
| | | suppressor of T cell | |
| | | activation (VISTA) | |
| Frizzled-10 (FZD10) | OTSA-101 | Integrin alpha-4/beta- | natalizumab |
| | | 1 | |
| GCSF-R (Also, | CSL324 | Immunoglobulin iota | anti-VPREB1 mAbs |
| CD114 and CSFR3) | | chain (VPREB1) | |
| Galectin 3 binding | MP-1959 | Wilms tumor protein | ESK1 |
| protein (LGALS3) | | (WT1/HLA); WT1 | |
| | | peptide presented in | |
| | | MHC | |
| Guanylate cyclase | TAK-164 | Glypican-3 (GPC3) | codrituzumab |
| 2C (GUCY2C) | | | |
| GD2 | dinutuximab | Transmembrane | CDX-011 |
| | | glycoprotein NMB | |
| | | (GPNMB) | |
| GD3 | PF-06688992 | Leucine-rich repeat- | BNC-101 |
| | | containing G-protein | |
| | | coupled receptor 5 | |
| | | (LGR5) | |
| glucocorticoid- | BMS-986156 | G-protein coupled | JNJ-64407564 |
| induced TNFR- | | receptor family C | |
| related protein | | group 5 member D | |
| (GITR) | | (GPRC5D) | |
| glucocorticoid- | EU-102 | Ferritin | Ferritarg P |
| induced TNFR- | | | |
| related protein | | | |
| ligand (GITRL) | | | |
| premelanocyte | anti-PMEL mAbs | Erbb2 tyrosine kinase | trastuzumab; |
| protein (PMEL) | | (HER2) | pertuzumab; |
| | | | margetuximab |
| Cell surface A33 | Anti-GPA33 mAbs | Erbb3 tyrosine kinase | patritumab |
| antigen (GPA33) | | (HER3) | |
| Glypican-1 (GPC1) | MIL-38 | Globo H | OBI-888 |

The extracellular antigen binding domain may comprise an antigen binding fragment (e.g., an scFv) derived from any of the antibodies listed in Table 2 depending upon the target antigen of interest. In some embodiments, the antigen binding fragment (e.g., an scFv) may comprise the same heavy chain and light chain complementarity determining regions (CDRs) as the antibodies listed in Table 2 depending upon the target antigen of interest. In some examples, the antigen binding fragment (e.g., an scFv) may comprise the same heavy chain variable region (VH) and light chain variable region VL as the antibodies listed in Table 2 depending upon the target antigen of interest.

In other embodiments, the extracellular antigen binding domain of any of the CAR polypeptides described herein may be specific to a pathogenic antigen, such as a bacterial antigen, a viral antigen, or a fungal antigen. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, coronavirus polypeptides, transmissible gastroenteritis glycoprotein 195, transmissible gastroenteritis matrix protein, human papilloma virus E6 or E7, or human hepatitis C virus glycoprotein E1 or E2.

In addition, the extracellular antigen binding domain of the CAR polypeptide described herein may be specific to a tag conjugated to a therapeutic agent, which targets an antigen associated with a disease or disorder (e.g., a tumor antigen or a pathogenic antigen as described herein). In some instances, the tag conjugated to the therapeutic agent can be antigenic and the extracellular antigen binding domain of the CAR polypeptide can be an antigen-binding fragment (e.g., scFv) of an antibody having high binding affinity and/or specificity to the antigenic tag. Exemplary antigenic tags include, but are not limited to, biotin, avidin, a fluorescent molecule (e.g., GFP, YRP, luciferase, or RFP), Myc, Flag, His (e.g., poly His such as 6×His), HA (hemagglutinin), GST, MBP (maltose binding protein), KLH (keyhole limpet hemocyanins), trx, T7, HSV, VSV (e.g., VSV-G), Glu-Glu, V5, e-tag, S-tag, KT3, E2, Au1, Au5, and/or thioredoxin.

In other instances, the tag conjugated to the therapeutic agent is a member of a ligand-receptor pair and the extracellular antigen binding domain comprises the other member of the ligand-receptor pair or a fragment thereof that binds the tag. For example, the tag conjugated to the therapeutic agent can be biotin and the extracellular antigen binding domain of the CAR polypeptide can comprise a biotin-binding fragment of avidin. See, e.g., (Urbanska et al. 2012, Lohmueller et al. 2017). Other examples include anti-Tag CAR, in which the extracellular antigen binding domain is a scFv fragment specific to a protein tag, such as FITC (Tamada et al. 2012, Kim et al. 2015, Cao et al. 2016, Ma et al. 2016), PNE (Ma et al. 2016), La-SS-B (Cartellieri et al. 2016), Biotin (Lohmueller et al. 2017) and Leucine-Zipper (Cho et al. 2018). Selection of the antigen binding domain for use in the CAR polypeptides described herein will be apparent to one of skill in the art. For example, it may depend on factors such as the type of target antigen and the desired affinity of the binding interaction.

The extracellular antigen binding domain of any of the CAR polypeptides described herein may have suitable binding affinity for a target antigen (e.g., any one of the targets described herein) or antigenic epitopes thereof. As used herein, “binding affinity” refers to the apparent association constant or K_{A} or the K_{D}. The K_{A} is the reciprocal of the dissociation constant (K_{D}). The extracellular antigen binding domain for use in the CAR polypeptides described herein may have a binding affinity (K_{D}) of at least 105, 10-6, 10-7, 10-8, 10-9, 10-10 M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 100,000-fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In one embodiment, the chimeric receptor polypeptide is a T-Cell Receptor (TCR) polypeptide, the TCR polypeptide comprising an extracellular domain, or portion thereof, of a TCR α chain, a TCR β chain, a TCR γ chain, a TCR δ chain, a CD3ε TCR subunit, a CD3γ TCR subunit, a CD3ζ TCR subunit or a CD3δ TCR subunit. In some instances, the TCR is further modified (e.g., express multiple TCRs against same or different target antigens or same or different epitopes on the same antigen or increasing affinity for the target or amino acid substitutions to increase preferential TCR chain pairing). When the TCR-expressing immune cells (e.g., TCR-expressing T cells, also called “TCR T cells”) are administered to a subject, they may enhance toxicity against cancer cells targeted by the TCR-CD3 complex via their binding to the peptide-MHC complex (Wieczorek et al. 2017, Shafer et al. 2022).

### T-Cell Receptor (TCR)

In one embodiment, the chimeric receptor polypeptide is a T-Cell Receptor (TCR) polypeptide, the TCR polypeptide comprising an extracellular domain, or portion thereof, of a TCR α chain, a TCR β chain, a TCR γ chain, a TCR δ chain, a CD3ε TCR subunit, a CD3γ TCR subunit, a CD3ζ TCR subunit or a CD3δ TCR subunit. In some instances, the TCR is further modified (e.g., express multiple TCRs against same or different target antigens or same or different epitopes on the same antigen or increasing affinity for the target or amino acid substitutions to increase preferential TCR chain pairing). When the TCR-expressing immune cells (e.g., TCR-expressing T cells, also called “TCR T cells”) are administered to a subject, they may enhance toxicity against cancer cells targeted by the TCR-CD3 complex via their binding to the peptide-MHC complex (Wieczorek et al. 2017, Shafer et al. 2022). Exemplary TCR polypeptides for use with the methods and compositions described herein are affinity matured TCRs or recombinant TCR-like molecules, but also naturally occurring TCRs. Typically they are, or are introduced as, α and β chains that dimerize within the cell and complex with endogenous CD3 components to form a functional TCR that redirects T cell specificity towards an antigen of interest as reviewed by Shafer et al. (2022). Respective CD3 components provided by the transfected immune cell are the CD3 chains ε, γ, δ and ζ forming together with the transfected α and β chains the T cell receptor that redirects the T cell towards an antigenic peptide presented at the cell surface by MHC molecules. In humans, antigen-presenting MHC alleles are broadly classified as HLA class I (A, B, or C), presenting predominantly cytosolic peptides, or HLA class II (DR, DP, or DQ), which predominantly present extracellular derived peptides. TCR modifications may be introduced to prevent mispairing and maximize surface expression.

Various modifications to TCRs are known in the art (Shafer et al. 2022). TCRs according to the invention may be murinized TCRs, where the human TCR constant regions is replaced with those of a mouse TCR constant region. Further, the addition of an extra disulfide bond in the TCR constant region through cysteine substitutions stabilizes interchain binding affinity of engineered TCR α/β chains while reducing their binding affinity with endogenous TCR α/β chains. The stability of the engineered TCR α chain can be increased through select hydrophobic substitutions in its transmembrane region. Domain swapped TCRs also exist, where large or specific segments of the engineered TCR α/β constant regions are inverted, which reduces propensity of engineered TCRs to mispair. Also single-chain TCRs (scTCR) are known in the art, which encode TCR antigen recognition and signaling domains into a single chain. Three-domain and two-domain scTCRs differ by the inclusion or absence of the TCR β constant region.

### Antibody-Coupled T Cell Receptor (ACTR)

In another embodiment, the chimeric receptor polypeptide is an antibody-coupled T cell Receptor (ACTR) protein, which can be expressed in an immune cell (e.g., an NK cell or a T cell). The ACTR protein containing an extracellular Fc-binding domain. When the ACTR-expressing immune cells are administered to a subject together with an anti-cancer antibody, they may enhance toxicity against cancer cells targeted by the antibody via their binding to the Fc domain of the antibody (Kudo et al. 2014). Accordingly, the chimeric receptor polypeptides may also be antibody-coupled T cell receptor (ACTR) polypeptides that comprise an Fc binding domain. The ACTR polypeptides described herein comprise an extracellular target binding domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or single domain antibodies (e.g., nanobodies). Alternatively, an Fc binding domain can be a synthetic peptide that specifically binds the Fc portion, such as a Kunitz domain, a small modular immunepharmaceutical (SMIP), an adnectin, an avimer, an affibody, a designed ankyrin repeat proteins (DARPin), or an anticalin, which may be identified by screening a peptide combinatory library for binding activities to Fc. In some embodiments, the Fc binding domain is an extracellular ligand-binding domain of a mammalian Fc receptor. As used herein, an “Fc receptor” is a cell surface bound receptor that is expressed on the surface of many immune cells (including B cells, T cells and NK cells and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc. List of a few exemplary polymorphisms in Fc receptor extracellular domains and exemplary Fc receptors constructing ACTR polypeptides are disclosed in WO2020010110A1, WO2020037066A1 and WO2020051493A1.

### Transmembrane Domain

The transmembrane domain of the chimeric receptor polypeptides (e.g., CAR polypeptides, TCR, or ACTR polypeptides) described herein can be in any form known in the art. As used herein, a “transmembrane domain” refers to any protein structure that is thermodynamically stable in a cell membrane, preferably a eukaryotic cell membrane. A transmembrane domain compatible for use in the chimeric receptor polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three-dimensional structure of the transmembrane domain. For example, transmembrane domains may form an alpha helix, a complex of more than one alpha helices, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-terminus.

In some embodiments, the transmembrane domain of the chimeric receptor polypeptide described herein is derived from a Type I single-pass membrane protein. Preferably, the transmembrane domain is of a membrane protein selected from the group consisting of CD8α, CD8β, 4-1BB/CD137, CD27, CD28, CD34, CD4, FccRIγ, CD16α, OX40/CD134, CD3ζ, CD3ε, CD3γ, CD3δ, TCRα, TCRβ, TCRγ, CD32, CD64, CD45, CD5, CD9, CD22, CD37, CD80, CD86, CD40, CD40L/CD154, VEGFR2, FAS, FGFR2B, CD2, IL-15R, DNAM-1, 2B4, NKG2D, NKp44 and NKp46.

In some embodiments, the transmembrane domain is from a membrane protein selected from the group consisting of CD8α, CD8β, 4-1BB, CD28, CD34, CD4, FcεRIγ, CD16α, OX40, CD3ζ, CD3ε, CD3γ, CD3δ, TCRα, CD32, CD64, VEGFR2, FAS, NKG2D, NKp44, NKp46 and FGFR2B. In some examples, the transmembrane domain is of CD8α (e.g., the transmembrane domain is of CD8α). In some examples, the transmembrane domain is of 4-1BB/CD137. In other embodiments, the transmembrane domain is of CD28. In other embodiments, the transmembrane domain is of NKG2D, NKp44 or NKp46. In other examples, the transmembrane domain is of CD34. In yet other examples, the transmembrane domain is not derived from human CD8α. In some embodiments, the transmembrane domain of the chimeric receptor polypeptide is a single-pass alpha helix. The amino acid sequences of exemplary transmembrane domains are provided in Table 3.

TABLE 3
Exemplary Transmembrane Domains

| Transmembrane | | SEQ |
|---|---|---|
| domain | Sequence | ID NO. |
| CD8α | DIYIWAPLAGTCGVLLLSLVITLYC | 6 |
| 4-1BB/CD137 | DIISFFLALTSTALLFLLFFLTLRFSVV | 7 |
| CD28 | DFWVLVVVGGVLACYSLLVTVAFIIFWVRS | 8 |
| CD34 | DLIALVTSGALLAVLGITGYFLMNR | 9 |
| Designed hydrophobic | DLLAALLALLAALLALLAALLARSK | 10 |
| CD4 | DMALIVLGGVAGLLLFIGLGIFFCVR | 11 |
| FCϵRIγ | DLCYILDAILFLYGIVLTLLYCRLK | 12 |
| Designed hydrophobic, | DLLLILLGVLAGVLATLAALLARSK | 13 |
| predicted dimerization | | |
| CD8β | DITLGLLVAGVLVLLVSLGVAIHLC | 14 |
| CD16α | DVSFCLVMVLLFAVDTGLYFSVKIN | 15 |
| OX40/CD134 | DVAAILGLGLVLGLLGPLAILLALY | 16 |
| CD3ϵ | DVMSVATIVIVDICITGGLLLLVYYWSKN | 17 |
| CD3ζ | DLCYLLDGILFIYGVILTALFLRVK | 18 |
| CD3γ | DGFLFAEIVSIFVLAVGVYF IAGQD | 19 |
| CD3δ | DGI IVTDVIATLLLALGVFCFAGHET | 20 |
| TCR-α | DVIGFRILLLKVAGENLLMTLRLW | 21 |
| CD32 | DIIVAVVIATAVAAIVAAVVALIYCRK | 22 |
| CD64 | DVLFYLAVGIMFLVNTVLWVTIRKE | 23 |
| VEGFR2 | DIIILVGIAVIAMFFWLLLVIILRT | 24 |
| FAS | DLGWLCLLLLPIPLIVWVKRK | 25 |
| FGFR2B | DIAIYCIGVFLIACMVVTVILCRMK | 26 |
| CD8 + 4aa | FACDIYIWAPLAGTCGVLLLSLVITLYC | 27 |

Transmembrane domains from multi-pass membrane proteins may also be compatible for use in the chimeric receptor polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. In some instances, the reverse orientation of such a native transmembrane protein may be constructed for efficient orientation of the chimeric receptor polypeptide (e.g., CAR) within the immune cell membrane. Either one or multiple helices passes from a multi-pass membrane protein can be used for constructing the chimeric receptor polypeptide described herein.

Transmembrane domains for use in the chimeric receptor polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906B1 and WO 2000/032776A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

### Cytoplasmic Signaling Domain

Any cytoplasmic signaling domain can be used to create the chimeric receptor polypeptides described herein (e.g., CAR polypeptides, TCRs, or ACTR polypeptides). Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells (e.g., CAR-T). The amino acid sequence of one exemplary cytoplasmic signaling domain of human CD3° C. is:

(SEQ ID NO: 28)
RVKFSRSADAPAYQQGQNQLYNELNLGRREEYDVLDKRRGRDP
EMGGKPRRKNPQEGLYNELQKDKMAEAYSEIGMKGERRRGKGH
DGLYQGLSTATKDTYDALHMQALPPR

In some embodiments, the cytoplasmic signaling domain is of CD3ζ or FcεRIγ. In a preferred embodiment, the cytoplasmic signaling domain is of CD3ζ. In other examples, cytoplasmic signaling domain is not derived from human CD3ζ. In yet other examples, the cytoplasmic signaling domain is not derived from an FcR, when the extracellular Fc-binding domain of the same chimeric receptor polypeptide is derived from CD16A.

In one specific embodiment, several signaling domains can be fused together for additive or synergistic effect. Non-limiting examples of useful additional signaling domains include part or all of one or more of TCRζ chain, CD28, OX40/CD134, 4-1BB/CD137, FcεRIγ, ICOS/CD278, IL2Rβ/CD122, IL-2Rγ/CD132, and CD40.

In some embodiments, the chimeric receptor polypeptide comprises one or more of the following features: (i) the chimeric receptor polypeptide further comprises a signal peptide at its N-terminus; (ii) the chimeric receptor polypeptide further comprises a hinge domain, which is located at the C-terminus of the extracellular target binding domain and the N-terminus of transmembrane domain; (iii) the chimeric receptor polypeptide is free of a hinge domain; (iv) the chimeric receptor polypeptide further comprises at least one co-stimulatory signaling domain; (v) the chimeric receptor polypeptide is free of a co-stimulatory signaling domain; and (vi) the cytoplasmic signaling domain comprises an immunoreceptor tyrosine-based activation motif (ITAM).

### Signal Peptide

In some embodiments, the chimeric receptor polypeptide (e.g., CAR, TCR or ACTR polypeptide) may also comprise a signal peptide (also known as a signal sequence) at the N-terminus of the polypeptide. Accordingly, the nucleic acid encoding the chimeric receptor polypeptide is also encoding a signal peptide, whereas in the mature polypeptide the signal peptide has been cleaved off. In general, signal sequences are peptide sequences that target a polypeptide to the desired site in a cell. In some embodiments, the signal sequence targets the chimeric receptor polypeptide to the secretory pathway of the cell and will allow for integration and anchoring of the chimeric receptor polypeptide into the lipid bilayer. Signal sequences including signal sequences of naturally occurring proteins or synthetic, non-naturally occurring signal sequences that are compatible for use in the chimeric receptor polypeptides described herein will be evident to one of skill in the art. In some embodiments, the signal sequence is from CD8α (10). In some embodiments, the signal sequence is from CD28 (11). In other embodiments, the signal sequence is from the murine kappa chain (12). In yet other embodiments, the signal sequence is from CD16. See Table 4.

TABLE 4
Exemplary Signal Peptides

| | | SEQ |
|---|---|---|
| | | ID |
| Signal Peptide | Sequence | NO. |
| CD8α | MALPVTALLLPLALLLHAARP | 29 |
| CD28 | MLRLLLALNLFPSIQVTG | 30 |
| Murine κ chain | METDTLLLWVLLLWVPGSTG | 31 |

### Hinge Domain

In some embodiments, the chimeric receptor polypeptides (e.g., CAR, TCR or ACTR polypeptide) described herein further comprise a hinge domain that is located between the extracellular ligand-binding domain and the transmembrane domain. A hinge domain is an amino acid segment that is generally found between two domains of a protein and may allow for flexibility of the protein and movement of one or both domains relative to one another. Any amino acid sequence that provides such flexibility and movement of the extracellular ligand-binding domain relative to the transmembrane domain of the chimeric receptor polypeptide can be used.

Hinge domains of any protein known in the art to comprise a hinge domain are compatible for use in the chimeric receptor polypeptides described herein. In some embodiments, the hinge domain is at least a portion of a hinge domain of a naturally occurring protein and confers flexibility to the chimeric receptor polypeptide. In one embodiment the chimeric receptor polypeptide comprises a hinge domain, which is a hinge domain selected from the list of CD28, CD16A, CD8, IgG, murine CD8α, and DAP12. In some embodiments, the hinge domain is of CD8α (e.g., the hinge domain is of CD8α). In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain stems from CD28. In some embodiments, the hinge domain is a portion of the hinge domain of CD28, e.g., a fragment containing at least 15 (e.g., 20, 25, 30, 35, or 40) consecutive amino acids of the hinge domain of CD28. The hinge domain and/or the transmembrane domain may be linked to additional amino acids (e.g., 15 aa, 10-aa, 8-aa, 6-aa, or 4-aa) at the N-terminal portion, at the C-terminal portion, or both. Examples can be found, e.g., in Ying et al. (2019).

In some embodiments, the hinge domain is of a CD16A receptor, for example, the whole hinge domain of a CD16A receptor or a portion thereof, which may consist of up to 40 consecutive amino acid residues of the CD16A receptor (e.g., 20, 25, 30, 35, or 40). Such a chimeric receptor polypeptide (e.g., an ACTR polypeptide) may contain no hinge domain from a different receptor (a non-CD16A receptor). In some cases, the chimeric receptor polypeptide described herein may be free of a hinge domain from any non-CD16A receptor. In some instances, such a chimeric receptor polypeptide may be free of any hinge domain.

Hinge domains of IgG antibodies, such as an IgG, IgA, IgM, IgE, or IgD antibodies, are also compatible for use in the chimeric receptor polypeptides described herein. In some embodiments, the hinge domain joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody, preferably IgG1 and IgG4. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptor polypeptides described herein. In some embodiments, the hinge domain between the C-terminus of the extracellular target-binding domain and the N-terminus of the transmembrane domain is a peptide linker, such as a (GlyxSer)ₙ linker, wherein x and n, independently can be an integer between 3 and 12, including 3, 4, 5, 6, 7, 8, 9, 10, 11, 12, or more. In some embodiments, the hinge domain is (Gly4Ser)ₙ, wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly4Ser)₃. In some embodiments, the hinge domain is (Gly4Ser)₆. In some embodiments, the hinge domain is (Gly4Ser)₉. In some embodiments, the hinge domain is (Gly4Ser)₁₂. In some embodiments, the hinge domain is (Gly4Ser)₁₅. In some embodiments, the hinge domain is (Gly4Ser)₃₀. In some embodiments, the hinge domain is (Gly4Ser)₄₅. In some embodiments, the hinge domain is (Gly4Ser)₆₀.

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the chimeric receptor polypeptide as described herein may contain up to 250 amino acid residues. In some instances, the chimeric receptor polypeptide may contain a relatively long hinge domain, for example, containing 150-250 amino acid residues (e.g., 150-180 amino acid residues, 180-200 amino acid residues, or 200-250 amino acid residues). In other instances, the chimeric receptor polypeptide may contain a medium sized hinge domain, which may contain 60-150 amino acid residues (e.g., 60-80, 80-100, 100-120, or 120-150 amino acid residues). In some instances, the hinge domain may be a flexible linker consisting of glycine and serine amino acids having a length between 15 and 60 amino acids, preferably composed of Gly4Ser units, especially one of the linkers of 24 to 26. Alternatively, the chimeric receptor polypeptide may contain a short hinge domain, which may contain less than 60 amino acid residues (e.g., 1-30 amino acids or 31-60 amino acids). In some embodiments, a chimeric receptor polypeptide (e.g., an ACTR polypeptide) described herein contains no hinge domain or no hinge domain from a non-CD16A receptor. The amino acid sequences of exemplary hinge domains are provided in

### Table 5:

TABLE 5
Exemplary Hinge Domains

| | | SEQ ID |
|---|---|---|
| Hinge domain | Sequence | NO. |
| CD8α | TTTPAPRPPTPAPTIASQPLSLRPEACRPAAGGAVHTRG | 32 |
| | LDFAC | |
| IgG1 (hinge- | EPKSCDKTHTCPPCPAPELLGGPSVFLFPPKPKDTLMIS | 33 |
| CH2-CH3) | RTPEVTCVVVDVSHEDPEVKFNWYVDGVEVHNAKTKPRE | |
| | EQYNSTYRVVSVLIVLHQDWLNGKEYKCKVSNKALPAPI | |
| | EKTISKAKGQPREPQVYTLPPSRDELTKNQVSLTCLVKG | |
| | FYPSDIAVEWESNGQPENNYKTTPPVLDSDGSFFLYSKL | |
| | TVDKSRWQQGNVFSCSVMHEALHNHYTQKSLSLSPGK | |
| IgG1 (hinge- | EPKSCDKTHTCPGQPREPQVYTLPPSRDELTKNQVSLTCL | 34 |
| CH3) | VKGFYPSDIAVEWESNGQPENNYKTTPPVLDSDGSFFLYS | |
| | KLTVDKSRWQQGNVFSCSVMHEALHNHYTQKSLSLSPGK | |
| IgG1 | EPKSCDKTHTCP | 35 |
| IgG4 | ESKYGPPCPPCP | 36 |
| CD8α-Fragment | TTTPAPRPPTPAPTIASQPLSLRPEAFAC | 37 |
| 1 (30 aa) | | |
| CD8α-Fragment | TTTPAPRPPTPFAC | 38 |
| 2 (15 aa) | | |
| CD28 | IEVMYPPPYLDNEKSNGTIIHVKGKHLCPSPLFPGPSKP | 39 |
| CD28 (39 aa) | IEVMYPPPYLDNEKSNGTIIHVKGKHLCPSPLFPGPSKP | 40 |
| CD28 (26 aa) | KSNGTIIHVKGKHLCPSPLFPGPSKP | 41 |
| CD28 (16 aa) | GKHLCPSPLFPGPSKP | 42 |
| (Gly₄Ser)ₓ₆ | GGGGSGGGGSGGGGSGGGGSGGGGSGGGGS | 43 |
| (30 aa) | | |
| (Gly₄Ser)ₓ₉ | GGGGSGGGGSGGGGSGGGGSGGGGSGGGGSGGGGSGGGGS | 44 |
| (45 aa) | GGGGS | |
| (Gly₄Ser)ₓ₁₂ | GGGGSGGGGSGGGGSGGGGSGGGGSGGGGSGGGGSGGGGS | 45 |
| (60 aa) | GGGGSGGGGSGGGGSGGGGS | |

In some embodiments, chimeric receptor polypeptides described herein may further comprise a hinge domain, which may be located at the C-terminus of the extracellular target binding domain and the N-terminus of the transmembrane domain. The hinge domain may be of any suitable length. In yet other embodiments, the chimeric receptor polypeptide described herein may have a shortened hinge domain (e.g., including up to 25 amino acid residues).

In other embodiments, the chimeric receptor polypeptide described herein is free of a hinge domain, i.e., does not have a hinge domain.

### Co-Stimulatory Signaling Domain (Co-Stimulatory Domain)

For many immune cells (e.g., NK or T cells) it is beneficial to include a co-stimulatory signaling domain for stimulation of an antigen-specific signal, to promote cell proliferation, differentiation and survival, as well as to activate effector functions of the cell. The term “co-stimulatory signaling domain”, as used herein, refers to at least a fragment of a co-stimulatory signaling protein that mediates signal transduction within a cell to induce an immune response such as an effector function (a secondary signal). As known in the art, activation of immune cells such as T cells often require two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A co-stimulatory receptor transduces a co-stimulatory signal (secondary signal) as an addition to the TCR-triggered signaling and modulates responses mediated by immune cells, such as T cells, NK cells, macrophages, neutrophils, or eosinophils.

Activation of a co-stimulatory signaling domain in an immune cell may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptor polypeptides described herein. The type(s) of co-stimulatory signaling domain is selected based on factors such as the type of the immune cells in which the chimeric receptor polypeptides would be expressed (e.g., T cells, NK cells, macrophages, neutrophils, or eosinophils) and the desired immune effector function (e.g., ADCC). Accordingly, it is in one embodiment that the chimeric receptor polypeptide of the genetically engineered immune cell comprises at least one co-stimulatory signaling domain. Examples of co-stimulatory signaling domains for use in the chimeric receptor polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4/CD2525, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP10, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2D, NKG2C, NKp30, NKp44, NKp46 and JAMAL. In certain embodiments, the chimeric receptor polypeptides may contain a CD28 co-stimulatory signaling domain or a 4-1BB (CD137) co-stimulatory signaling domain. In some embodiments, at least one co-stimulatory signaling domain is selected from the group consisting of CD8α, CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL, or any functional variant thereof.

Also within the scope of the present disclosure are functional variants of any of the co-stimulatory signaling domains described herein, such that the co-stimulatory signaling domain is capable of modulating the immune response of the immune cell. In some embodiments, the co-stimulatory signaling domains comprise up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor polypeptide. In some embodiments, the mutations are substitution of a lysine at each of positions 186 and 187 with a glycine residue of the CD28 co-stimulatory domain, referred to as a CD28LL-GG variant. Therefore, a suitable variant of CD28 is the CD28LL-GG variant.

Additional mutations can be made in co-stimulatory signaling domains that may enhance or reduce co-stimulatory activity of the domain will be evident to one of ordinary skill in the art. In some embodiments, the co-stimulatory signaling domain is selected from the group of 4-1BB, CD28, OX40, and CD28LL-GG variant.

In some embodiments, the chimeric receptor polypeptides may contain a single co-stimulatory domain such as, for example, a CD27 co-stimulatory domain, a CD28 co-stimulatory domain, a 4-1BB co-stimulatory domain, an ICOS co-stimulatory domain, an OX40 co-stimulatory domain, an OX40L co-stimulatory domain, a 2B4 co-stimulatory domain, a GITR co-stimulatory domain, a NKG2D co-stimulatory domain, a NKp30 co-stimulatory domain, a NKp44co-stimulatory domain, a NKp46 co-stimulatory domain, a DAP10 co-stimulatory domain, a DAP12 co-stimulatory domain, a DNAMI co-stimulatory domain, a LFA-1 co-stimulatory domain, a HVEM co-stimulatory domain or a JAMAL co-stimulatory domain. In one embodiment, the at least one co-stimulatory signaling domain is a CD28 co-stimulatory signaling domain or a 4-1BB co-stimulatory signaling domain.

Selection of the type(s) of co-stimulatory signaling domains may be based on factors such as the type of immune cells (e.g., αβ T, yd T or NK cells) to be used with the chimeric receptor polypeptides and the desired immune effector function.

In some embodiments, the chimeric receptor polypeptides may comprise more than one co-stimulatory signaling domain (e.g., 2, 3, or more). In one embodiment, the chimeric receptor polypeptide comprises at least two co-stimulatory signaling domains. In one preferred embodiment, the chimeric receptor polypeptide comprises two co-stimulatory signaling domains. In some embodiments, the chimeric receptor polypeptide comprises two or more of the same co-stimulatory signaling domains, for example, two copies of the co-stimulatory signaling domain of CD28. In some embodiments, the chimeric receptor polypeptide comprises two or more co-stimulatory signaling domains from different co-stimulatory proteins, such as any two or more co-stimulatory proteins described herein. In some embodiments, the chimeric receptor polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28LL-GG variant and 4-1BB, CD28 and OX40, or CD28LL-GG variant and OX40. In some embodiments, the two co-stimulatory signaling domains are CD28 and 4-1BB. In some embodiments, the two co-stimulatory signaling domains are CD28LL-GG variant and 4-1BB. In some embodiments, the two co-stimulatory signaling domains are CD28 and OX40. In some embodiments, the two co-stimulatory signaling domains are CD28LL-GG variant and OX40. In some embodiments, the chimeric receptor polypeptides described herein may contain a combination of a CD28 and ICOSL. In some embodiments, the chimeric receptor polypeptide described herein may contain a combination of CD28 and CD27. In certain embodiments, the 4-1BB co-stimulatory domain is located N-terminal to the CD28 or CD28LL-GG variant co-stimulatory signaling domain.

In some embodiments, one of the co-stimulatory signaling domains is a CD28 co-stimulatory signaling domain and the other co-stimulatory domain is selected from the group consisting of a CD8α, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. In some embodiments, one of the co-stimulatory signaling domains is a CD8α co-stimulatory signaling domain and the other co-stimulatory domain is selected from the group consisting of a CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. In some embodiments, one of the co-stimulatory signaling domains is a 4-1BB co-stimulatory signaling domain and the other co-stimulatory domain is selected from the group consisting of a CD8α, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. The amino acid sequences of exemplary co-stimulatory signaling domains are provided in Table 6.

TABLE 6
Exemplary Co-Stimulatory Signaling Domains

| Co-stimulatory | | SEQ ID |
|---|---|---|
| domain | Sequence | NO. |
| 4-1BB/CD137 | KRGRKKLLYIFKQPFMRPVQTTQEEDGCSCRFPEEEEGG | 46 |
| | CEL | |
| CD28 | RSKRSRLLHSDYMNMTPRRPGPTRKHYQPYAPPRDFAAY | 47 |
| | RS | |
| OX40/CD134 | ALYLLRRDQRLPPDAHKPPGGGSFRTPIQEEQADAHSTL | 48 |
| | AKI | |
| OX40L/CD252 | ERVQPLEENVGNAARPRFERNK | 49 |
| ICOS | KKKYSSSVHDPNGEYMFMRAVNTAKKSRLTDVTL | 50 |
| CD27 | QRRKYRSNKGESPVEPAEPCHYSCPREEEGSTIPIQEDY | 51 |
| | RKPEPACSP | |
| GITR | QLGLHIWQLRSQCMWPRETQLLLEVPPSTEDARSCQFPE | 52 |
| | EERGERSAEEKGRLGDLWV | |
| HVEM | CVKRRKPRGDVVKVIVSVQRKRQEAEGEATVIEALQAPP | 53 |
| | DVTTVAVEETIPSFTGRSPNH | |
| TIM1 | KKYFFKKEVQQLSVSFSSLQIKALQNAVEKEVQAEDNIY | 54 |
| | IENSLYATD | |
| LFA1/CD11a | DIYIWAPLAGTCGVLLLSLVITLYCYKVGFFKRNLKEKM | 55 |
| | EAGRGVPNGIPAEDSEQLASGQEAGDPGCLKPLHEKDSE | |
| | SGGGKD | |
| CD2 | DIYIWAPLAGTCGVLLLSLVITLYCKRKKQRSRRNDEEL | 56 |
| | ETRAHRVATEERGRKPHQIPASTPQNPATSQHPPPPPGH | |
| | RSQAPSHRPPPPGHRVQHQPQKRPPAPSGTQVHQQKGPP | |
| | LPRPRVQPKPPHGAAENSLSPSSN | |
| CD28 + CD27 | RSKRSRLLHSDYMNMTPRRPGPTRKHYQPYAPPRDFAA | 57 |
| | YRSQRRKYRSNKGESPVEPAEPCHYSCPREEEGSTIPI | |
| | QEDYRKPEPACSP | |
| CD28 + OX40 | RSKRSRLLHSDYMNMTPRRPGPTRKHYQPYAPPRDFAA | 58 |
| | YRSRRDQRLPPDAHKPPGGGSFRTPIQEEQADAHSTLA | |
| | KI | |
| 4-1BB + CD28 | KRGRKKLLYIFKQPFMRPVQTTQEEDGCSCRFPEEEEG | 59 |
| | GCELRSKRSRLLHSDYMNMTPRRPGPTRKHYQPYAPPR | |
| | DFAAYRS | |
| CD28 + 4-1BB | RSRLLHSDYMNMTPRRPGPTRKHYQPYAPPRDFAAYRS | 60 |
| | KRGRKKLLYIFKQPFMRPVQTTQEEDGCSCRFPEEEEG | |
| | GCEL | |
| CD28 + ICOS | RSKRSRLLHSDYMNMTPRRPGPTRKHYQPYAPPRDFAA | 61 |
| | YRSKKKYSSSVHDPNGEYMFMRAVNTAKKSRLTDVTL | |
| CD28LL to GG | RSKRSRGGHSDYMNMTPRRPGPTRKHYQPYAPPRDFAA | 62 |
| mutant | YRS | |
| CD28LL to GG | RSKRSRGGHSDYMNMTPRRPGPTRKHYQPYAPPRDFAA | 63 |
| mutant + 4-1BB | YRSKRGRKKLLYIFKQPFMRPVQTTQEEDGCSCRFPEE | |
| | EEGGCEL | |
| DAP10 | CARPRRSPAQEDGKVYINMPGRG | 64 |
| DNAM-1 | NRRRRRERRDLFTESWDTQKAPNNYRSPISTSQPTNQS | 65 |
| | MDDTREDIYVNYPTFSRRPKTRV | |
| NKp30 | GSTVYYQGKCLTWKGPRRQLPAVVPAPLPPPCGSSAHL | 66 |
| | LPPVPGG | |
| NKp44 | WWGDIWWKIMMELRSLDTQKATCHLQQVTDLPWTSVSS | 67 |
| | PVEREILYHTVARTKISDDDDEHTL | |

The optional co-stimulatory signaling domain may be located in the cytoplasm for triggering activation and/or effector signaling.

In some instances, the chimeric receptor polypeptide is free of any co-stimulatory signaling domain.

### Immunoreceptor Tyrosine-Based Activation Motif (ITAM)

The cytoplasmic signaling domain described herein may comprise an immunoreceptor tyrosine-based activation motif (ITAM) domain (e.g., at least one ITAM domain, at least two ITAM domains, or at least three ITAM domains). An “ITAM,” as used herein, is a conserved protein motif that is generally present in the tail portion of signaling molecules expressed in many immune cells. The motif may comprise two repeats of the amino acid sequence YxxL/I separated by 6-8 amino acids, wherein each x is independently any amino acid, producing the conserved motif YxxL/Ix₍₆₋₈₎ YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Examples of ITAMs for use in the chimeric receptor polypeptides comprised within the cytoplasmic signaling domain, without limitation may be: CD3γ, CD3ε, CD3δ, each containing a single ITAM motif while each (chain contains 3 distinct ITAM domains (ζa, ζb and ζc). The number and ITAM sequences are also important in the design of CARs (Bettini et al. 2017, Jayaraman et al. 2020). Examples include, but are not limited to, the cytoplasmic signaling domain of Jak/STAT, Toll-interleukin receptor (TIR), and tyrosine kinase.

In other embodiments, the cytoplasmic signaling domain described herein is free of the ITAM motif.

In some embodiments, a chimeric receptor polypeptide as described herein may comprise, from N-terminus to C-terminus, the extracellular target binding domain, the transmembrane domain, and the cytoplasmic signaling domain. In some embodiments, a chimeric receptor polypeptide as described herein comprises, from N-terminus to C-terminus, the extracellular target binding domain, the transmembrane domain, at least one co-stimulatory signaling domain, and the cytoplasmic signaling domain. In other embodiments, a chimeric receptor polypeptide as described herein comprises, from N-terminus to C-terminus, the extracellular target binding domain, the transmembrane domain, the cytoplasmic signaling domains, and at least one co-stimulatory signaling domain.

In a preferred embodiment the CAR comprises, from N- to C-terminus, a CD8α signal peptide, an scFv against a tumor target of choice, an IgG4 hinge domain, a CD28 transmembrane domain, a 4-1BB co-stimulatory signaling domain and a CD33 cytoplasmic signaling domain.

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8α transmembrane domain, a CD8α hinge domain, or a combination thereof. In some embodiments, the CAR polypeptides may further include one or more co-stimulatory signaling domains, one of which may be a CD28 co-stimulatory signaling domain or a 4-1BB co-stimulatory signaling domain. In other examples, a CAR polypeptide described herein may comprise (i) a CD28 co-stimulatory domain or a 4-1BB co-stimulatory domain, (ii) a CD28 transmembrane domain, a CD8α hinge domain, or a combination thereof.

In an exemplary embodiment, the CAR polypeptide comprises (i) a CD8α hinge domain (ii) a CD8α transmembrane domain, (iii) a CD28 co-stimulatory domain or a 4-1BB co-stimulatory domain, (iv) a CD3ζ cytoplasmic signaling domain or a combination thereof. In other embodiments, the CAR polypeptide comprising two co-stimulatory domains further comprises (i) a CD8α or CD28 hinge domain (ii) a CD8α or CD28 transmembrane domain, (iii) a CD28 co-stimulatory domain or a 4-1BB co-stimulatory domain, (iv) a OX40L co-stimulatory domain, a 2B4 co-stimulatory domain, a DAP10 co-stimulatory domain, a DNAM-1 co-stimulatory domain, a NKG2D co-stimulatory domain, a NKp30 co-stimulatory domain, a NKp44 co-stimulatory domain, a NKp46 co-stimulatory domain or a JAMAL co-stimulatory domain, or (v) a CD3 cytoplasmic signaling domain or a combination thereof. In another exemplary embodiment, the CAR polypeptide comprising two co-stimulatory domains further comprises (i) a CD8α hinge domain (ii) a CD28 transmembrane domain, (iii) a CD28 co-stimulatory domain or a 4-1BB co-stimulatory domain, (iv) a OX40L co-stimulatory domain, a 2B4 co-stimulatory domain, a DAP10 co-stimulatory domain, a DNAM-1 co-stimulatory or a JAMAL co-stimulatory domain, or (v) a CD3ζ cytoplasmic signaling domain or a combination thereof.

In another exemplary embodiment, the CAR polypeptide comprising two co-stimulatory domains further comprises (i) a CD8α hinge domain (ii) a CD28 transmembrane domain, a NKp44 transmembrane domain, a NKG2D transmembrane domain or a NKp46 transmembrane domain, (iii) a CD28 co-stimulatory domain, a 4-1BB co-stimulatory domain, a 2B4 co-stimulatory domain or a DAP10 co-stimulatory, (iv) an OX40L co-stimulatory domain, a 2B4 co-stimulatory domain, a DAP10 co-stimulatory domain, a DAP12 co-stimulatory domain, a DNAM-1 co-stimulatory domain or a JAMAL co-stimulatory domain, or (v) a CD3 cytoplasmic signaling domain, a DAP12 cytoplasmic signaling domain or a 2B4 cytoplasmic signaling domain or a combination thereof. In an exemplary embodiment, the CAR polypeptide comprises (i) a CD8α hinge domain (ii) a CD28 transmembrane domain, (iii) a CD28 co-stimulatory domain or a 4-1BB co-stimulatory domain, (iv) an OX40L co-stimulatory domain or an OX40 co-stimulatory domain, (v) a CD3ζ cytoplasmic signaling domain or a combination thereof. For example, the CAR polypeptide may comprise an amino acid sequence selected from SEQ ID NO: 70 or SEQ ID NO: 71 provided below.

The CAR polypeptides are configured such that, when expressed on an immune cell (e.g., T or NK cell), the extracellular antigen-binding domain is located extracellularly for binding to a target molecule (e.g., a tumor antigen) and the CD3ζ cytoplasmic signaling domain is located intracellularly for signaling into the cell. The co-stimulatory signaling domain may be located in the cytoplasm for triggering activation and/or effector signaling. In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40L co-stimulatory signaling domain, an OX40 co-stimulatory signaling domain, a CD27 co-stimulatory signaling domain, a 2B4 co-stimulatory signaling domain or an ICOS co-stimulatory signaling domain), and the CD3ζ cytoplasmic signaling domain. Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3ζ cytoplasmic signaling domain, refers to any portion of a protein (protein X) that interacts with the interior of a cell or organelle and is capable of relaying a primary signal as known in the art, which leads to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

### IL-2/IL-15Rβγ Agonists

As described above, IL-2/IL-15Rβγ agonist refers to a class of molecules or complexes that have the agonistic targeting of the joint receptor of IL-2 and IL-15, the IL-2/IL-15Rβγ, and a decreased or abandoned binding of the IL-2Rα or IL-15Rα in common. They induce intracellular signaling through JAK1 (Janus kinase 1), JAK 3, and STAT3/5 (signal transducer and activator of transcription 3 and 5).

Accordingly, IL-2 derivatives or variants have been generated that reduce/abolish the binding to the IL-2α receptor chain in order to reduce the undesirable effects of traditional IL-2 therapies, such as vascular leak syndrome and expansion of immunosuppressive regulatory T cells (T_{regs}), while maintaining the stimulation of cytotoxic CD8⁺ T cells and NK cells. Similarly, a number of IL-15-based therapies are being developed, based on its capability to activate both the innate immune response through NK cells and the adaptive immune response through CD8⁺ T cells (Steel et al. 2012, Conlon et al. 2019), recently reviewed by Knudson (Knudson et al. 2020).

One way to modulate the IL-15 activity to target the IL-2/IL-15Rβγ is to mimic IL-15 trans-presentation by antigen presenting cells by complexing IL-15 with the sushi domain of the IL-15Rα. Examples for this concept are SOT101 (RLI-15, SO-C101), N-803 (ALT-803) and NIZ985 (hetIL-15) already contain (part of) the IL-15Rα subunit. SOT101 binds with high affinity to the mid-affinity IL-15Rβγ, as it comprises the covalently attached sushi+ domain of IL-15Rα. In turn, SOT101 does not bind/has relatively reduced binding to IL-2Rαβγ and IL-15Rαβγ. Similarly, ALT-803 and hetIL-15 carry an IL-15Rα sushi domain or the soluble IL-15Rα, respectively, and therefore can bind with high affinity to the mid-affinity IL-15Rβγ receptor. However, due to their non-covalent binding there is a chance that the complex dissociates in vivo and thereby the dissociated fraction of the applied complex further exerts other binding. Probability for dissociation is likely higher for ALT-803 vs. hetIL-15, as ALT-803 only comprises the sushi domain of IL-15Rα, which is known to mediate only partial binding to IL-15, whereas the sushi+ domain is required for full binding (Wei et al. 2001).

Another example of targeting mid-affinity IL-2/IL-15Rβ receptors is NKTR-214, whose hydrolyzation to its most active 1-PEG-IL-2 state generates a species whose location of PEG chains at the IL-2/IL-2Rα interface interferes with binding to the high-affinity IL-2Rα, while leaving binding to the mid-affinity IL-2/IL-15Rβ unperturbed (Charych et al. 2016). Further, the mutant IL-2 IL2v with abolished binding to the IL-2Rα subunit is an example of this class of compounds (Klein et al. 2013, Bacac et al. 2016). The targeting of the mid-affinity IL-2/IL-15Rβγ receptor avoids liabilities associated with targeting the high-affinity IL-2 and IL-15 receptors such as T_{reg} activation induced by IL-2 or vascular leakage syndrome which can be induced by high concentrations of soluble IL-2 or IL-15.

This is due to the fact that the IL-2Rαβγ high affinity receptor is additionally expressed on CD4⁺ T_{regs} and vascular endothelium and is activated by IL-2 cis-presentation. Therefore, compounds targeting (also) the high-affinity IL-2Rα By potentially lead to T_{reg} expansion and vascular leak syndrome (VLS), as observed for native IL-2 or soluble IL-15 (Conlon et al. 2019). Potentially VLS can be also caused by the de-PEGylated NKTR-214. De-PEGylated NKT2-214 has however a short half-life and it needs to be seen in the clinical development whether at all or to which extent this side-effect plays a role.

The high-affinity IL-15Rαβγ receptors activated by IL-15 cis-presentation are constitutively expressed in T cell leukemia and upregulated on inflammatory NK cells, inflammatory CD8⁺ T cells and Fibroblast-like synoviocytes (Kurowska et al. 2002, Perdreau et al. 2010), i.e., these cells also express the IL-15Rα subunit. Such activation should be avoided because of the IL-15 cis-presentation on these cells is involved in the development of T cell leukemia and exacerbation of the immune response, potentially triggering autoimmune diseases. Similarly, the high-affinity IL-15Rαβγ receptor is expressed on vascular endothelium and soluble IL-15 can also induce VLS. IL-15/IL-15Rα complexes do not bind to this high-affinity receptor as they already carry at least the sushi domain of the IL-15Rα, which sterically hinders the binding to the heterotrimeric IL-15Rαβγ receptor. These side effects triggered via engagement of high affinity IL-15Rαβγ receptors are triggered by native IL-15, but also by non-covalent IL-15/IL-15Rα complexes such as ALT-803 and hetIL-15, if disintegration of the complexes occurs in vivo.

Finally, the high-affinity IL-15Rα is constitutively expressed on myeloid cells, macrophages, B cells and neutrophils (Chenoweth et al. 2012) and may be activated by native IL-15 and again by non-covalent IL-15/IL-15Rα complexes such as ALT-803 and hetIL-15, if disintegration of the complexes occurs in vivo.

Another motivation of modifying IL-2 or IL-15 is the short in vivo half-life of the cytokines. Most of the designed IL-2/IL-15Rβγ agonists therefore aim for increasing their in vivo half-life either by fusing the IL-15, IL-2 or variant thereof to another protein, e.g., to the soluble IL-15Rα (hetIL-15, where the complexation with the receptor goes along with a considerable extension of the half-life), to add an Fc part of an antibody to the complex (ALT-803) or IL-15/IL-15Rα Fc fusions (P-22339) disclosed in U.S. Pat. No. 10,206,980 and IL-15/IL-15Rα heterodimeric Fc-fusions with extended half-lives (Bernett et al. 2017) (WO 2014/145806), to a non-binding IgG (IgG-IL2v) or to an albumin binding domain (see WO 2018/151868A2). Other examples of IL-2/IL-15Rβγ agonists are CT101-IL2 (Ghasemi et al. 2016, Lazear et al. 2017), PEGylated IL-2 molecules like and NKTR-214 (Charych et al. 2016) and THOR-924 (Caffaro et al. 2019) (WO 2019/028419, WO 2019/028425), the polymer-coated IL-15 NKTR-255 (Miyazaki et al. 2018), NL-201/NEO-201 (Silva et al. 2019), RGD-targeted IL-15/IL-15Rα Fc complex (US 2019/0092830), RTX-240 by Rubius Therapeutics (red blood cells expressing an IL-15/IL-15Rα fusion protein, WO 2019/173798), and THOR-707 (Joseph et al. 2019).

Studies indicated that ALT-803 has a 7.5-hour serum half-life in mice (Liu et al. 2018) and 7.2 to 8 h in cynomolgus monkeys (Rhode et al. 2016) compared with <40 minutes observed for IL-15 (Han et al. 2011). In the clinic, ALT-803 was administered i.v. or s.c. in a Phase I dose escalation trial weekly for 4 consecutive weeks, followed by a 2-week rest period for continued monitoring, for two 6-week cycles of therapy starting at 0.3 μg/kg up to 20 μg/kg. Results from the trial led to the selection of 20 μg/kg/dose s.c. weekly as the optimal dose and route of delivery for ALT-803 (Margolin et al. 2018).

NKTR-214 is described as a highly “combinable cytokine” dosed more like an antibody than a cytokine due to its long half-life in vivo. Its anticipated dosing schedule in humans is once every 21 days. Yet NKTR-214 provides a mechanism of direct immune stimulation characteristic of cytokines. PEGylation dramatically alters the pharmacokinetics of NKTR-214 compared with IL-2, providing a 500-fold increase in AUC in the tumor compared with an IL-2 equivalent dose. Pharmacokinetics of NKTR-214 were determined after i.v. administration in mice and resulted for the most active species of NKTR-214 (i.e. 2-PEG-IL2, 1-PEG-IL2, free IL2) in a gradually increase, reaching Cₘₐₓ at 16 hours post dose and a decrease with t½ of 17.6 hours (Charych et al. 2017). Based on the increased half-life due to PEGylation, NKTR-214 was tested in five dose regimens in combination with nivolumab in NCT02983045 (see clinicaltrials.gov)
0.006 mg/kg NKTR-214 every 3 weeks (q3w) with 240 mg nivolumab every two weeks (q2w),
0.003 mg/kg NKTR-214 q2w with 240 mg nivolumab q2w,
0.006 mg/kg NKTR-214 q2w with 240 mg nivolumab q2w,
0.006 mg/kg NKTR-214 q3w with 360 mg nivolumab q3w,
0.009 mg/kg NKTR-214 q3w with 360 mg nivolumab q3w.

After completion of the first part of the study it was continued with a dose of 0.006 mg/kg NKTR-214 q3w with 360 mg nivolumab q3w.

IL-2/IL-15 mimetics have been designed by a computational approach, which is reported to bind to the IL-2Rβγ heterodimer but have no binding site for IL-2Rα (Silva et al. 2019) and therefore also qualify as IL-2/IL-15Rβγ agonists. Due to their small size of about kDa (see supplementary information Figure S13) they are expected to have a rather short in vivo half-life.

However, already less than 15 min exposure of cells with IL-15 (at 10 ng/ml) expressing the receptor to native IL-15 leads to the maximal level of Stat5 activation and subsequent pharmacodynamic effects (Castro et al. 2011).

Accordingly, the IL-2/IL-15Rβγ agonists may have at least one of the following features:
1) Reduced binding to the IL-2Rαβγ for IL-2-derived agonists, or IL-15Rα By for IL-15-derived agonists;
2) Comprising the sushi domain of IL-2 for IL-2-derived agonists, or the sushi domain of IL-15 for IL-15-derived agonists;
3) Having an increased half-life compared to human IL-2 for IL-2 derived agonists, or to human IL-15 for IL-15 derived agonists, through fusion of IL-2 or a derivative thereof or IL-15 or a derivative thereof to another polypeptide, e.g. the sushi domain of IL-15 for IL-15-derived agonists, an Fc part of an antibody, or albumin, or PEG,
4) Having a masked binding to IL-2/IL-15 receptors, which is demasked in the tumor environment.

Reduced binding may be achieved by introducing amino acid substitutions into the IL-2 or the IL-15. Respective mutation are known in the art, e.g., as done for IL2v (Klein et al. 2013, Bacac et al. 2016) (WO 2012/107417A1) by mutation of L72, F42 and/or Y45, especially F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, for example, mutations F42A, Y45A and L72G, R37A and F41K in MK-1484, or C125S in MDNA11 for IL-2Rα, or L44D, E46K, L47D, V49D, 150D, L66D, L66E, 167D, and I67E, for reducing binding to the IL-15Rα (WO 2016/142314A1), or N1, N4, D8, D30, D61, E64, N65, and Q108 for gradually reducing the activity of the respective IL-15/IL-15Rα complex regarding activating of NK cells and CD8 T cells (see FIG. 51, WO 2018/071918A1, WO 2018/071919A1). In another embodiment, site-specific PEGylation, that sterically hinders the binding to the respective Rx may be used generate IL-2/IL-15Rβγ agonists. Examples are THOR-707 (Joseph et al. 2019) (WO2019/028419A1), NKTR-214, NKTR-255, THOR-924, -908, -918, TransCon IL-2β/γ. Also WO 2016/060996A2 defines specific regions of IL-15 as being suitable for substitutions (see para. 0020, 0035, 00120 and 00130) and specifically provides guidance how to identify potential substitutions for providing an anchor for a PEG or other modifications (see para. 0021).

Examples of IL-2/IL-15Rβγ agonists comprising a sushi domain are ALKS 4230 for IL-2 derived agonists, and SOT101, ALT-803, NIZ985, P-22339 and XmAb24306 for IL-15 derived agonists.

Examples of IL-2/IL-15Rβγ agonists with an increased half-life are ALKS 4230, SOT101, ALT-803, NIZ985, P-22339 and XmAb24306, THOR-707 (Joseph et al. 2019) (WO2019/028419A1), NKTR-214, NKTR-255, THOR-924, -908, -918, and TransCon IL-2β/γ

Examples of masked IL-2/IL-15Rβγ agonists are XTX202, WTX-124.

In a preferred embodiment of the present invention the IL-2/IL-15βγ agonist is a complex comprising a polypeptide comprising the amino acid sequence of the interleukin 15 (IL-15) or a derivative thereof, and a polypeptide comprising the amino acid sequence of the sushi domain of an interleukin 15-receptor alpha (IL-15Rα) or a derivative thereof. The IL-2/IL-15Rβγ agonist according to the invention comprise an IL-15 or derivative thereof and at least the sushi domain of the IL-15Rα or derivative thereof. They target the mid-affinity IL-2/IL-15Rβγ, i.e., the receptor consisting of the IL-2/IL-15Rβ and the γ_{c} subunits, which is expressed on NK cells, CD8⁺ T cells, NKT cells and γδ T cells. These complexes are well-known in the art, and their binding capabilities are well understood. In some examples, the complex comprises a human IL-15 (SEQ ID NO: 55) or a derivative thereof, and the sushi domain of the human IL-15Rα (SEQ ID NO: 57), the sushi+ fragment/domain of the human IL-15Rα (SEQ ID NO: 58) or a soluble form of the human IL-15Rα (from amino acids 31 to either of amino acids 172, 197, 198, 199, 200, 201, 202, 203, 204 or 205 of SEQ ID NO: 56, see WO 2014/066527, (Giron-Michel et al. 2005)). Additionally, the enlarged size leads to an increased in vivo half-life. Examples are SOT101, ALT-803, NIZ985, P-22339 and XmAb24306.

In a preferred embodiment, the IL-2/IL-15Rβγ agonist comprises the polypeptides comprising the amino acid sequence of human IL-15 or a derivative thereof, wherein the IL-derivative has an amino acid sequence having a percentage of identity of at least 92.5% with the amino acid of SEQ ID NO: 55 (mature human IL-15), and the polypeptide comprising the sushi domain of the human IL-15Rα or a derivative thereof, wherein the sushi domain derivative has an amino acid sequence having a percentage of identity of at least 92% with the amino acid sequence of SEQ ID NO: 57 (sushi domain of human IL-15Rα). In some specific examples, the sushi domain may comprise the amino acid of SEQ ID NO: 58 (sushi+ fragment of human IL-15Rα). One specific example is an IL-2/IL-15Rβγ agonist comprising an IL-15 derivative with an amino acid sequence having a percentage of identity of at least 92.5% with the amino acid of SEQ ID NO: 55 and comprising the sushi domain of the human IL-15Rα with the amino acid of SEQ ID NO: 57.

In some embodiments, the polypeptides of the complex are covalently linked in a fusion protein, which results in a stable complex that does not dissociate in vivo, when the concentration of the complex becomes low. In such a fusion protein the amino acid sequence of the IL-15 or derivatives thereof is optionally in a C-terminal position relative to the amino acid sequence of the sushi domain of the IL-15Rα or derivatives thereof.

In a further embodiment, the amino acid sequence of the IL-15 or derivatives thereof and the amino acid sequence of the sushi domain of the IL-15Rα or derivatives are separated by a linker amino acid sequence. The linker is preferably flexible, preferably has a length of 2-30 amino acids, more preferably of 10-30 amino acids, more preferably of 15-30 amino acids, still more preferably of 15-25 amino acids, most preferably of 18-22 amino acids. G and S are preferred amino acids for the flexible linker. A suitable linker is provided as SEQ ID NO: 59. In some examples, the human IL-15 comprises the sequence of SEQ ID NO: 55 and the human IL-15Rα sushi domain comprises the sequence of SEQ ID NO: 57, more preferably comprising the sushi+ fragment/domain of SEQ ID NO: 58. Such fusion protein is, in some instances, in the order (from N- to C-terminus) IL-15Rα-linker-IL-15 (RLI-15). An especially preferred IL-2/IL-15Rβγ agonist is the fusion protein designated SOT101 (RLI2) having the sequence of SEQ ID NO: 60.

Other formats of IL-15/IL-15Rα agonist/fusion proteins are also encompassed by the invention. The IL-15 and the IL-15Rα sushi domain-containing polypeptide may covalently be linked through an introduced disulfide bond, e.g., by introduced L52C substation in IL-15 and S40C substation in the sushi domain. Further, the IL-15 and the IL-15Rα may be independently fused to a carrier protein/dimerizing carrier proteins, e.g., a heterodimeric Fc fusion protein as described in US 2018/0118805 (para. 0161 to 0228), where IL-15 is bound to the N-terminus of one Fc chain and the IL-15Rα sushi domain is bound to the N-terminus of a second Fc chain to form a heterodimeric agonist through the disulfide bonds of the Fc domains.

Accordingly, further preferred IL-2/IL-15Rβγ agonists are P-22339 are XmAb24306. P-22339 refers to an IL-2/IL-15Rβγ agonist, where IL-15 is bound to the N-terminus of one Fc chain and the IL-15Rα sushi domain is bound to the N-terminus of a second Fc chain as described in WO 2016/095642 and Hu et al. (2018) with the L52C substitution on the IL-15 polypeptide (SEQ ID NO: 64) and the S40C substitution on the IL-15Rα sushi+ polypeptide (SEQ ID NO: 65) forming a disulfide bond. XmAb24306 refers to an IL-15/IL-15Rα sushi agonist, where a mutant IL-15 is bound to the N-terminus of one Fc chain and the IL-15Rα sushi domain is bound to the N-terminus of a second Fc chain as described in as described in US 2018/0118805 (see XENP024306 in FIG. 94C, SEQ ID NO: 66 and SEQ ID NO: 67).

In one example, the IL-2/IL-15Rβγ agonist is a half-life extended agonist. Half-life extension is partly leading to the improved stimulation by the IL-2/IL-15Rβγ agonist according to this invention, as the prolonged half-life leads to a stronger activation of NK and CD8⁺ T cells. Half-life extension is achieved basically by increasing the size of the molecule, e.g., to other polypeptides like albumin or the Fc part of an antibody, or by conjugation to chemical moieties known to extend the half-life like multiple polyethylene glycol units (PEG).

In a specific embodiment, the IL-2/IL-15Rβγ agonist comprises (i) a polypeptide comprising the amino acid sequence of an IL-15 derivatives having at least 0.1% of the activity of human IL-15 on the proliferation induction of kit225 cells, and (ii) a polypeptide comprising the amino acid sequence of the sushi domain of IL-15Rα or derivatives thereof having at least 10% of the binding activity of the sushi domain of human IL-15Rα to human IL-15. In one embodiment, the IL-15 derivative has only at least 0.01 of the activity of human IL-15 on the proliferation induction of kit225 cells. The (in vitro) activity of IL-15 can be determined by induction of proliferation of kit225 cells as described by Hori et al. (1987). Kit225 cells (Hori et al. 1987) are passaged in kit225 base medium and used for the potency assay at passage 4-7. Before the potency assay, kit225 cells are cultivated in kit225 base medium without IL-2 for 24 h (starvation period). 1×10⁴ kit225 cells are plated in 96-well plate and a serial dilution of test IL-15 compounds added to cells. Cells are incubated at 37° C., 5% CO₂ for 72±3 h. Following the incubation, 10 μl (10% of the volume in the well) of Alamar Blue is added to each well and, after 6 h, absorbance is measured at 560 nm with a 620 nm reference using a Tecan Spark absorbance microplate reader (set mixing before detection for 15 s). In some cases, when lower potency of IL-15 mutants is tested, the incubation with kit225 cells should be prolonged from 3 days (72 h±3 h) to 5 days. Preferably, methods such as colorimetry or fluorescence are used to determine proliferation activation due to IL-15 stimulation, as for example described by Soman et al. using CTLL-2 cells (Soman et al. 2009). As an alternative to cell lines such as the kit225 cells, human peripheral blood mononuclear cells (PBMCs) or buffy coats can be used. A preferred bioassay to determine the activity of IL-15 is the IL-2/IL-15 Bioassay Kit using STAT5-RE CTLL-2 cells (Promega Catalog number CS2018B03/B07/B05).

In some instances, an IL-15 derivative has at least 0.1% of the activity of human IL-15, preferably 1%, more preferably at least 10%, more preferably at least 25%, even more preferably at least 50%, and most preferably at least 80%. Interleukins are extremely potent but short-lived molecules, even such low activities as 0.1% of human IL-15 may still be sufficiently potent, especially if dosed higher or if an extended half-life compensates for the loss of activity. A reduced binding of a mutant IL-15 or a mutant IL-2/IL-15Rβγ agonist to the IL-2/IL-15Rβγ receptor results in a lower target-mediated drug deposition and therefore to a longer in vivo half-life. Accordingly, the longer in vivo half-life with its increased AUC of the agonist compensates for the lower in vitro activity.

In one embodiment said IL-15 derivative has at least 25% of the activity of human IL-15 on the proliferation induction of kit225 cell line, more preferably at least 50%, and has an amino acid sequence having a percentage of identity of at least 92.5% with an amino acid sequence of SEQ ID NO: 55, preferably of at least 96%, and more preferably of at least 98.5% or of at least 99% identiy.

In analogy, in one embodiment said IL-2 derivative has at least 25% of the activity of human IL-2 on the proliferation induction of kit225 cell line, more preferably at least 50%, and has an amino acid sequence having a percentage of identity of at least 92.5% with an amino acid sequence of SEQ ID NO: 50, preferably of at least 96%, and more preferably of at least 98.5% or of at least 99% identiy

In another embodiment, said derivative of the sushi domain of IL-15Rα has at least 25%, more preferably at least 50% of the binding activity of the sushi domain of human IL-15Rα, and has an amino acid sequence having a percentage of identity of at least 92.5% with an amino acid sequence selected in the group comprising SEQ ID NO: 57 or SEQ ID NO: 58, preferably of at least 96%, and more preferably of at least 98% identity.

### Administration

The immune cell population of the invention and the IL-2/IL-15Rβγ agonist of the invention are manufactured as separate pharmaceutical compositions, each being formulated with respective buffers and excipients. Accordingly, they are administered separately, i.e., they are not mixed into a single dose. Each is administered at an effective dose. In some embodiments, an effective dose of the immune cell population and an effective dose of the agonist are administered sequentially, more preferably the agonist is administered after the immune cell population. In one embodiment, the agonist is administered at least about 5 days, but not more than 21 days after the immune cell population.

Without being bound by any theory, it is expected that the immune cell population comprising immune effector cells especially for the treatment of solid tumors over a couple of days lose (part of) their tumoricidal activity due to one or more of the factors of upregulation of PD-1, entering/staying in the immune-suppressive tumor microenvironment, suffering from energy deprivation in the tumor microenvironment, and getting exhausted.

The administered agonist is then capable of reactivating such immune effector cells and thereby boosting their activity. In order to conveniently match clinical routines, the agonist is administered about 7 days after the administration of the immune cell population, preferably the agonist is administered in treatment cycles starting at about 7 days after the administration of the immune cell population. Treatment cycles for agonist typically comprise the administration for one or multiple times within a treatment period of two to three weeks, e.g., once or twice per week for 2 or 3 weeks, followed by a period of, e.g., 1 or 2 weeks without agonist administration for resetting the immune system. Then a new cycle can begin. Treatment cycles are continued over several months or until disease progression.

In one specific example, the agonist and the immune cell population are administered sequentially, wherein the agonist is administered using a cyclical administration regimen, wherein the cyclical administration regimen comprises:
(a) a first period of “x” days during which the agonist is administered at a daily dose on “y” consecutive days at the beginning of the first period followed by “x-y” days without administration of the agonist,
wherein “x” is 7, 14, or 21 days, preferably 7 days and “y” is 1, 2, 3 or 4 consecutive days, preferably 1 or 2 days;
(b) repeating the first period at least once; and
(c) a second period of “z” days without administration of the agonist, wherein z is 7, 14, or 21 days, preferbaly 7 or 14 days.

In some embodiments, this cyclic administration regimen begins at least 5 days, preferably 7 days after the administration of the immune cell population. The cycle is typically repeated at least two times, preferably at least three times, more preferably until disease progression. Repeated administration is especially preferred for NK cell therapies. Optionally, for multiple administrations of the immune cell population, this cyclic administration of the agonist is merged with the repeated administration of the immune cell population, so that prior to each start of agonist cycle there is one administration of the immune cell population.

Alternatively or in addition, the agonist is administered at a daily dose of 4.4 μmol/kg to 2200 μmol/kg, preferably 22 μmol/kg to 660 μmol/kg, and more preferably 220 μmol/kg to 530 μmol/kg. For SOT101 having a molecular weight of 22.7 kDa, the agonist is administered at a daily dose of 0.1 μg/kg to 50 μg/kg, preferably 0.5 μg/kg to 15 μg/kg, and more preferably 5 μg/kg to 12 μg/kg.

The pharmacodynamic effect of the administration can be easily determined by the activation of proliferation of immune cells, e.g., NK cells in blood. Accordingly, in one embodiment the administration of the agonist results in:
(a) an increase of the % of Ki-67⁺ NK of total NK cells in comparison to no administration of the agonist, and wherein the administration of the agonist after at least one repetition of the first period (step (b) results in a Ki-67⁺ NK cell level that is at least 70% of the of the Ki-67⁺ NK cells;
(b) maintenance of NK cell numbers or preferably an increase of NK cell numbers to at least 110% as compared to no administration of agonist after at least one repetition of the first period (step (b)); and/or
(c) NK cell numbers of at least 1.1×10³ NK cells/μl after at least one repetition of the first period. (step (b).

In one embodiment, the immune cell population is administered to the subject at a dose of at least about 1×10⁴ immune cells/kg, preferably from about 1×10⁴ to about 1×10¹² cells/kg. In accordance with the present disclosure, patients can be treated by infusing therapeutically effective doses of the immune cell population as described here in the range of about 10⁵ to 10¹⁰ or more cells per kilogram of body weight (cells/kg). The infusion can be repeated as often and as many times as the patient can tolerate until the desired response is achieved, to be aligned with the dosing schedule of the IL-2/IL-15Rβγ agonist as disclosed supra. For example, in a 4-week dosing schedule the immune cell population is administered at day 1, the agonist is then administered at days 8, 9, 15, and 16 (i.e., twice per week at two following days for two weeks starting one week after the immune cell administration, followed by one week of treatment break). The next cycle starting again with the administration of the immune cells then begins at day 29 corresponding to day 1 of the first cycle.

The appropriate infusion dose and schedule will vary from patient to patient, but can be determined by the treating physician for a particular patient. Typically, initial doses of approximately 10⁶ cells/kg will be infused, escalating to 10⁸ or more cells/kg.

Other co-treatment typically used for tumor patients may be combined with the combination treatment of this invention. It is especially foreseen that the treatment of tumor patients further comprises administering:
(a) a lymphocyte reduction treatment, preferably selected from cyclophosphamide and fludarabine;
(b) a further therapeutic agent; and/or
(c) a further anti-tumor treatment.

Lymphocyte reduction treatment, or lymphodepletion, is routinely applied prior to the administration of the immune cell population in order to reduce the number of circulating immune cells to allow expansion of the administered immune cells. In some cases, there is no lymphodepletion treatment. Further therapeutic agents may be agents that patients receive to treat comorbidities, pain and a like. In tumor therapy, typically different anti-tumor treatments are combined. These encompass chemotherapy with known cytotoxic or cytostatic compounds, radiation therapy, targeted therapies like kinase inhibitors, anti-tumor antibodies, bispecific antibodies or antibody-drug-conjugates, immunotherapies like immune checkpoint inhibitors, or other compounds approved for tumor therapy.

The combination therapy of this invention can be applied in principle to all tumor diseases and is especially suitable for solid tumors, where the negative effect of the tumor microenvironment so far has limited the clinical success of adoptive cell therapies. Therefore, in one embodiment of the invention the subject is a human patient suffering from a cancer which is a solid tumor or liquid tumor. A solid tumor may be selected from the group consisting of carcinoma, lymphoma, sarcoma and blastoma.

Cancer may be selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, mesothelioma, pancreatic cancer, head and neck cancer, retinoblastoma, glioma, glioblastoma, liver cancer, and thyroid cancer; optionally wherein the cancer of B-cell origin is selected from the group consisting of Hodgkin lymphoma and non-Hodgkin lymphoma.

In a preferred embodiment, the patient is suffering from a solid tumor. More preferably, the solid tumor has a tumor microenvironment having a low glucose level. In a preferred embodiment, the cancer is not a hematologic malignancy, i.e., blood cancers, which impact the normal production and function of blood cells, including the three categories of leukemias, lymphomas, and multiple myeloma.

In vivo Transduction

The invention further relates to the embodiment of a viral particle suitable for transducing an immune cell as defined supra comprising a vector genome comprising a polynucleotide sequence encoding a chimeric receptor polypeptide as defined supra for use in treatment of cancer in a subject in combination with an IL-15/IL-15Rα conjugate as defined supra. The manufacture of the immune cell populations for adoptive therapy requires complex manufacturing processes, complex logistics and, in the autologous setting, induce a delay for treatment, which increases the risk for the patient while waiting for the treatment. Accordingly, transducing the immune cells in vivo with an off-the-shelf viral particle product carrying the polynucleotides encoding the chimeric receptor polypeptide overcomes these shortcomings. However, as the number of transduced immune cells may be relatively low, it is further advantageous to further treat the patient with the IL-15/IL-15Rα conjugate in order to lead to a further proliferation of transduced immune cells, increase the viability and/or increase the anti-tumor cytotoxicity of the transduced immune cells in the tumor microenvironment.

In a preferred embodiment, the viral particles are retroviral particles or lentiviral particles. Respective viral particles are disclosed in WO 2019/200056A2, WO 2020/106992A1, WO 2022/164935A1, WO 2022/183072A1, and WO 2023/015217A1.

In a preferred embodiment, the immune cell is a T cell. WO 2023/015217A1 discloses lentiviral vectors comprising a CD4 binding agent to specifically target CD4⁺ T cells. WO 2022/183072A1 discloses lentiviral vectors further encoding heterologous lymphocyte targeting protein specifically targeting T cells or NK cells. Examples for heterologous lymphocyte targeting proteins are:
(a) a T cell marker selected from CD3, CD28, CD80, 4-1BB, AhR, CD3, CD2, CD7, CD4, CD8, CD25, CD44, CD45RA, CD47, CD62L, CD69, CD94, CD95, CD127, CD161, CD183 (CXCR3), CD184 (CXCR4), CD185 (CXCR5), CD193 (CCR3), CD194 (CCR4), CD195 (CCR5), CD196 (CCR6), CD197 (CCR7), CCR10, PD-1, TCRa/b, CD5, CD27, CD45RO, CD45RB, CD57, CD103, CD122, P2RX7, TIGIT, LAG-3, TIM-3, and IL6ST, or any combination thereof;
(b) a γδ T cell marker selected from yo TCR, Vdelta1, Vdelta2, and NKG2D (KLRK1, CD314); or
(c) an NK T cell marker selected from Invariant TCR (Va24-Ja18), CD185 (CXCR5), CXCR6, and IL-21R, or any combination thereof.

In one embodiment, the polynucleotide sequence further encodes a metabolism modulating polypeptide as defined supra. The same advantages of the expression or overexpression of a polypeptide that (a′) improves glucose uptake activity; (b′) modulates the Krebs cycle; (c′) enhances lactate concentration; or (d′) diverts or redirects glucose metabolites out of a glycolysis pathway applies to the immune cells transduced in vivo, and therefore, the combination of expressing or overexpressing metabolism modulating polypeptides with co-treatment with IL-15/IL-15Rα conjugates are equally beneficial.

The viral particle for in vivo transduction may be administered transdermally, intravenously, intratumorally, intraperitoneally, intramuscularly, intranodally or subcutaneously. In a preferred embodiment, the viral particle is for intravenous, intraperitoneal, subcutaneous or intranodal injection. The viral particles are preferably formulated for such administration with conventional buffers and/or excipients as known to a person skilled in the art.

In addition to in vivo transduction using viral particles, non-viral in vivo transduction methods are envisaged, as these have the potential to make cellular therapies more cost-effective and avoid risks associated with viral transduction such as undesired immune responses, pre-existing immunity or dysregulation of oncogenes by viral integration. A review of presently used non-viral delivery systems is provided by Mendes et al. (2022) highlighting liposomes, lipid nanoparticles (LNP) and polymeric nanoparticles: LNPs typically comprise ionizable and cationic lipids, cholesterol, phospholipids and PEG lipids having a size of 50 to 100 nm in diameter. The helper lipids (e.g. phospholipids and cholesterol) stabilize the formulation and enhance membrane fusion. Polymeric nanoparticles are produced from natural polymers, e.g. dextran, chitosan and/or cyclodextrins, or synthetic polymers, e.g. poly-L-lysine, polyethyleneimine, polyamidoamine or poly(lactic-co-glycolic acid) in the form of nanocapsules or nanospheres. The cationic polymers (e.g., polyethyleneimine, chitosan or biodegradable polyesters) bind and condense the nucleic acid through electrostatic interactions. Liposomes are based on phospholipids with polar head groups and non-polar tails (e.g. cationic: N-[1-(2,3-dioleoyloxy) propyl]-N,N,N-trimethylammonium chloride (DOTMA), 1,2-dioleoyl-3-trimethylammonium propane (DOTAP) and zwitterionic: 1,2-dioleoyl-sn-glycero-3-phosphoethanolamine (DOPE)) along with stabilizers (e.g. cholesterol), which spontaneously assemble into vesicles. In addition, inorganic nanoparticles such as gold nanoparticles, silica nanoparticles or iron oxide nanoparticles may be used.

Accordingly, a further embodiment of the invention is a nucleic acid or a nucleic acid set comprising a polynucleotide sequence encoding a chimeric receptor polypeptide according to the invention for use in treatment of cancer in a subject in combination with a conjugate of the invention. Preferably, the nucleic acid is a vector suitable for transient or stable transduction of an immune cell as described in the section “Vectors” above. The nucleic acid, the set of nucleic acids or the vector according to the invention may be formulated for non-viral transduction according to non-viral transduction methods/delivery systems known in the art. Preferred non-viral transduction systems/formulations are LNPs, liposomes, polymeric nanoparticles and inorganic nanoparticles. For LNPs, the formulation comprises the nucleic acid, the set of nucleic acids or the vectors of the invention, and ionizable and cationic lipids, cholesterol, phospholipids and PEG lipids. For liposomes, the formulation comprises the set of nucleic acids or the vectors of the invention, and phospholipids with polar head groups and non-polar tails, e.g., N-[1-(2,3-dioleoyloxy) propyl]-N,N,N-trimethylammonium chloride (DOTMA), 1,2-dioleoyl-3-trimethylammonium propane (DOTAP) or 1,2-dioleoyl-sn-glycero-3-phosphoethanolamine (DOPE)) and a stabilizer, e.g. cholesterol. For polymeric nanoparticles, the formulation comprises the set of nucleic acids or the vectors of the invention, and a natural polymer, e.g. dextran, chitosan and/or cyclodextrins, or a synthetic polymer, e.g. poly-L-lysine, polyethyleneimine, polyamidoamine or poly(lactic-co-glycolic acid).

### Method of Treatment

The invention further relates to a method for inhibiting and/or killing tumor cells in a subject, the method comprising administering to a subject in need thereof an immune cell population in combination with an IL-2/IL-15Rβγ agonist as defined herein, wherein an effective dose of the immune cell population and an effective dose of the agonist are administered sequentially.

The invention further relates to a method for increasing the viability of a previously administered immune cell population in the tumor microenvironment by subsequently administering an IL-2/IL-15Rβγ agonist as defined herein.

The invention further relates to a method for increasing the anti-tumor cytotoxicity of a previously administered immune cell population in the tumor microenvironment by subsequently administering an IL-2/IL-15Rβγ agonist as defined herein.

The invention further relates to a method for inhibiting and/or killing tumor cells in a subject, the method comprising administering to a subject in need thereof a therapeutically effective amount of viral particles as defined supra, a nucleic acid as defined supra, a vector as defined supra, or a non-viral transduction system as defined supra, wherein the viral particles, the nucleic acid, the vector or the non-viral transduction system are suitable for transducing immune cells in vivo, in combination with an IL-15/IL-15Rα conjugate as defined supra, wherein a therapeutically effective dose of the viral particles, the nucleic acid, the vector or the non-viral transduction system and a therapeutically effective dose of the conjugate are administered sequentially.

The invention further relates to a method for increasing the viability of in vivo transduced immune cells in the tumor microenvironment after administration of viral particles, the nucleic acid, the vector or the non-viral transduction system as defined in supra in a subject by subsequently administering an IL-15/IL-15Rα conjugate as defined supra.

The invention further relates to a method for increasing the anti-tumor cytotoxicity of in vivo transduced immune cells in the tumor microenvironment after administration of viral particles, the nucleic acid, the vector or the non-viral transduction system as supra in a subject by subsequently administering an IL-15/IL-15Rα conjugate as defined supra.

The efficacy of the combined treatments or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the metabolism modulating polypeptides that redirect glucose metabolites and the CAR polypeptides) in the subject, for example, by the overall health of the subject and/or the presence of adverse events or severe adverse events.

```
SEQUENCES
human IL-2
SEQ ID NO: 68
    1 MYRMQLLSCI ALSLALVINS APTSSSTKKT QLQLEHLLLD LQMILNGINN YKNPKLTRML
  61 TFKFYMPKKA TELKHLQCLE EELKPLEEVL NLAQSKNFHL RPRDLISNIN VIVLELKGSE
121 TTEMCEYADE TATIVEFLNR WITFCQSIIS TLT 153
mature human IL-2
SEQ ID NO: 69
                          APTSSSTKKT QLQLEHLLLD LQMILNGINN YKNPKLTRML
  61 TFKFYMPKKA TELKHLQCLE EELKPLEEVL NLAQSKNFHL RPRDLISNIN VIVLELKGSE
121 TTEMCEYADE TATIVEFLNR WITFCQSIIS TLT 153
IL2v
SEQ ID NO: 70
    1 APASSSTKKT QLQLEHLLLD LQMILNGINN YKNPKLTRML
  41 TAKFAMPKKA TELKHLQCLE EELKPLEEVL NGAQSKNFHL RPRDLISNIN VIVLELKGSE
101 TTEMCEYADE TATIVEFLNR WITFAQSIIS TLT
NEO 2-15 E62C
SEQ ID NO: 71
PKKKIQLHAEHALYDALMILNIVKINSPPAEEKLEDYAFNFELILEEIARLFESGDQKDE
ACKAKRMKEWMKRIKTTASEDEQEEMANAIITILQSWIES
MDNA11: IL-2_{C125S} albumin fusion
SEQ ID NO: 72
MYRMQLLSCIALSLALVINSAPTSSSTKKTQLQLEHLLLDLQMILNGINNYKNPKLTRMLTAKFYMPKKATELKH
LQCLEEALKPLEEVLNLAQSKNFHFDPRDVVSNINVFVLELKGSETTEMCEYADETATIVEFLNRWITFSQSIIS
TLTGGGGSGGGGSGGGGSDAHKSEVAHRFKDLGEENFKALVLIAFAQYLQQCPFEDHVKLVNEVTEFAKTCVADE
SAENCDKSLHTLFGDKLCTVATLRETYGEMADCCAKQEPERNECFLQHKDDNPNLPRLVRPEVDVMCTAFHDNEE
TFLKKYLYEIARRHPYFYAPELLFFAKRYKAAFTECCQAADKAACLLPKLDELRDEGKASSAKQRLKCASLQKFG
ERAFKAWAVARLSQRFPKAEFAEVSKLVTDLTKVHTECCHGDLLECADDRADLAKYICENQDSISSKLKECCEKP
LLEKSHCIAEVENDEMPADLPSLAADFVESKDVCKNYAEAKDVELGMELYEYARRHPDYSVVLLLRLAKTYETTL
EKCCAAADPHECYAKVEDEFKPLVEEPQNLIKQNCELFEQLGEYKFQNALLVRYTKKVPQVSTPTLVEVSRNLGK
VGSKCCKHPEAKRMPCAEDYLSVVLNQLCVLHEKTPVSDRVTKCCTESLVNRRPCESALEVDETYVPKEENAETT
FHADICTLSEKERQIKKQTALVELVKHKPKATKEQLKAVMDDFAAFVEKCCKADDKETCFAEEGKKLVAASQAAL
GL
human IL-15
SEQ ID NO: 73
    1 MRISKPHLRS ISIQCYLCLL LNSHELTEAG IHVFILGCES AGLPKTEANW VNVISDLKKI
  61 EDLIQSMHID ATLYTESDVH PSCKVTAMKC FLLELQVISL ESGDASIHDT VENLIILANN
121 SLSSNGNVTE SGCKECEELE EKNIKEFLQS FVHIVQMFIN TS 162
mature human IL-15
SEQ ID NO: 74
                                                          NW VNVISDLKKI
  61 EDLIQSMHID ATLYTESDVH PSCKVTAMKC FLLELQVISL ESGDASIHDT VENLIILANN
121 SLSSNGNVTE SGCKECEELE EKNIKEFLQS FVHIVQMFIN TS
162
human IL-15Rα
SEQ ID NO: 75
    1 MAPRRARGCR TLGLPALLLL LLLRPPATRG ITCPPPMSVE HADIWVKSYS LYSRERYICN
  61 SGFKRKAGTS SLTECVLNKA TNVAHWTTPS LKCIRDPALV HQRPAPPSTV TTAGVTPQPE
121 SLSPSGKEPA ASSPSSNNTA ATTAAIVPGS QLMPSKSPST GTTEISSHES SHGTPSQTTA
181 KNWELTASAS HQPPGVYPQG HSDTTVAIST STVLLCGLSA VSLLACYLKS RQTPPLASVE
241 MEAMEALPVT WGTSSRDEDL ENCSHHL
sushi domain of human IL-15Rα
SEQ ID NO: 76
CPPPMSVE HADIWVKSYS LYSRERYICN SGFKRKAGTS SLTECVLNKA TNVAHWTTPS LKC
sushi+ fragment of human IL-15Rα
SEQ ID NO: 77
ITCPPPMSVE HADIWVKSYS LYSRERYICN SGEKRKAGTS SLTECVLNKA TNVAHWTTPS
LKCIRDPALV HORPAPP
linker
SEQ ID NO: 78
SGG SGGGGSGGGS GGGGSGG
SOT101 (RLI2)
SEQ ID NO: 79
    1 ITCPPPMSVE HADIWVKSYS LYSRERYICN SGFKRKAGTS SLTECVLNKA INVAHWTTPS
  61 LKCIRDPALV HQRPAPPSGG SGGGGSGGGS GGGGSGGNWV NVISDLKKIE DLIQSMHIDA
121 TLYTESDVHP SCKVTAMKCF LLELQVISLE SGDASIHDTV ENLIILANNS LSSNGNVTES
181 GCKECEELEE KNIKEFLQSF VHIVQMFINT S 211
Leader peptide of (IL-15_{N72D})₂:IL-15Rαₛᵤₛₕᵢ-Fc:
SEQ ID NO: 80
METDTLLLWV LLLWVPGSTG
IL-15Rαₛᵤₛₕᵢ (65 aa)-Fc (IgG1 CH2-CH3):
SEQ ID NO: 81
    1 ITCPPPMSVE HADIWVKSYS LYSRERYICN SGFKRKAGTS SLTECVLNKA TNVAHWTTPS
  61 LKCIREPKSC DKTHTCPPCP APELLGGPSV FLFPPKPKDI LMISRTPEVT CVVVDVSHED
120 PEVKFNWYVD GVEVHNAKTK PREEQYNSTY RVVSVLTVLH QDWLNGKEYK CKVSNKALPA
180 PIEKTISKAK GQPREPQVYT LPPSRDELTK NQVSLTCLVK GFYPSDIAVE WESNGQPENN
240 YKTTPPVLDS DGSFFLYSKL TVDKSRWQQG NVFSCSVMHE ALHNHYTOKS LSLSPGK
IL-15_{N72D}
SEQ ID NO: 82
                                                          NW VNVISDLKKI
  61 EDLIQSMHID ATLYTESDVH PSCKVTAMKC FLLELQVISL ESGDASIHDT VENLIILAND
121 SLSSNGNVTE SGCKECEELE EKNIKEFLQS FVHIVQMFIN TS
IL-15L52C
SEQ ID NO: 83
NWVNVISDLKKIEDLIQSMHIDATLYTESDVHPSCKVTAMKCFLLELQVISCESGDASIHDTVENLIILANNSLS
SNGNVTESGCKECEELEEKNIKEFLQSFVHIVQMEINTS
IL-15Rα-sushi+_{S40C}-Fc
SEQ ID NO: 84
ITCPPPMSVE HADIWVKSYS LYSRERYICN SGEKRKAGTC SLTECVLNKA TNVAHWTTPS
LKCIRDPALV HQRGGGGSGG GGSEPKSSDK THTCPPCPAP ELLGGPSVEL FPPKPKDTLM
ISRIPEVTCV VVDVSHEDPE VKENWYVDGV EVHNAKTKPR EEQYNSTYRV VSVLIVLHQD
WLNGKEYKCK VSNKALPAPI EKTISKAKGQ PREPQVYTLP PSREEMTKNQ VSLTCLVKGF
YPSDIAVEWE SNGQPENNYK TTPPVLDSDG SEFLYSKLTV DKSRWQQGNV FSCSVMHEAL
HNHYTQKSLS LSPGK
XENP024306 chain 1: human IL-15 D₃₀N/E₆₄Q/N₆₅D (GGGGS)₁-
Fc(216)_IgG1_pI(-) Isosteric A C2205/PVA_/S₂₆₇K/L₃₆₈D/K₃₇₀S/M₄₂₈L/N₄₃₄S
SEQ ID NO: 85
NWVNVISDLKKIEDLIQSMHIDATLYTESNVHPSCKVTAMKCELLELQVISLESGDASIHDTVQDLIILANNSLS
SNGNVTESGCKECEELEEKNIKEFLQSFVHIVQMEINTSGGGGSEPKSSDKTHTCPPCPAPPVAGPSVFLFPPKP
KDTLMISRTPEVTCVVVDVKHEDPEVKENWYVDGVEVHNAKTKPREEEYNSTYRVVSVLIVLHQDWLNGKEYKCK
VSNKALPAPIEKTISKAKGQPREPQVYTLPPSREEMTKNQVSLTCDVSGFYPSDIAVEWESDGQPENNYKTTPPV
LDSDGSFFLYSKLTVDKSRWEQGDVFSCSVLHEALHSHYTQKSLSLSPGK
XENP024306 chain 2: human IL15Rα(sushi) (GGGGS)₁-
Fc(216)_IgG1_C2205/PVA_/S₂₆₇K/S₃₆₄K/E₃₅₇Q/M₄₂₈L/N₄₃₄S
SEQ ID NO: 86
ITCPPPMSVEHADIWVKSYSLYSRERYICNSGFKRKAGTSSLTECVLNKATNVAHWTTPSLKCIRGGGGSEPKSS
DKTHTCPPCPAPPVAGPSVFLFPPKPKDTLMISRTPEVTCVVVDVKHEDPEVKENWYVDGVEVHNAKTKPREEQY
NSTYRVVSVLTVLHQDWLNGKEYKCKVSNKALPAPIEKTISKAKGQPREPQVYTLPPSREQMTKNQVKLTCLVKG
FYPSDIAVEWESNGQPENNYKTTPPVLDSDGSFFLYSKLTVDKSRWQQGNVFSCSVLHEALHSHYTQKSLSLSPG
K
anti-ROR-1 CAR
SEQ ID NO: 87
MALPVTALLLPLALLLHAARPQEQLVESGGRLVTPGGSLTLSCKASGEDESAYYMSWVRQAPGKGLEWIATIYPS
SGKTYYATWVNGRFTISSDNAQNTVDLQMNSLTAADRATYFCARDSYADDGALFNIWGPGTLVTISSGGGSGGGG
SGGGGSELVLTQSPSVSAALGSPAKITCTLSSAHKIDTIDWYQQLQGEAPRYLMQVQSDGSYTKRPGVPDRESGS
SSGADRYLIIPSVQADDEADYYCGADYIGGYVFGGGTQLTVTGESKYGPPCPPCPFWVLVVVGGVLACYSLLVTV
AFIIFWVKRGRKKLLYIFKQPFMRPVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMKGERRRGKGHDGLYQGLSTATKDTY
DALHMQALPPR
Bold/Italic: CD8a signal peptide
Plain: anti-ROR-1 scFv (from EP 2884999)
Bold: IgG4 hinge
italic: CD28 transmembrane domain
Underlined: 4-1BB (CD137) co-stimulatory domain
Bold underlined: cytoplasmic signaling domain of human CD3ζ
anti-ROR-1 CAR and GOT2
SEQ ID NO: 88
MALPVTALLLPLALLLHAARPQEQLVESGGRLVTPGGSLTLSCKASGEDESAYYMSWVRQAPGKGLEWIATIYPS
SGKTYYATWVNGRFTISSDNAQNTVDLQMNSLTAADRATYFCARDSYADDGALENIWGPGTLVTISSGGGSGGGG
SGGGGSELVLTQSPSVSAALGSPAKITCTLSSAHKIDTIDWYQQLQGEAPRYLMQVQSDGSYTKRPGVPDRESGS
SSGADRYLIIPSVQADDEADYYCGADYIGGYVFGGGTQLTVTGESKYGPPCPPCPFWVLVVVGGVLACYSLLVTV
AFIIFWVKRGRKKLLYIFKQPFMRPVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMKGERRRGKGHDGLYQGLSTATKDTY
DALHMQALPPRGSGATNFSLLKQAGDVEENPGPMALLHSGRVLPGIAAAFHPGLAAAASARASSWWTHVEMGPPD
PILGVTEAFKRDTNSKKMNLGVGAYRDDNGKPYVLPSVRKAEAQIAAKNLDKEYLPIGGLAEFCKASAELALGEN
SEVLKSGRFVTVQTISGTGALRIGASFLQRFFKFSRDVFLPKPTWGNHTPIFRDAGMQLQGYRYYDPKTCGFDET
GAVEDISKIPEQSVLLLHACAHNPTGVDPRPEQWKEIATVVKKRNLFAFFDMAYQGFASGDGDKDAWAVRHFIEQ
GINVCLCQSYAKNMGLYGERVGAFTMVCKDADEAKRVESQLKILIRPMYSNPPLNGARIAAAILNTPDLRKQWLQ
EVKVMADRIIGMRTQLVSNLKKEGSTHNWQHITDQIGMFCFTGLKPEQVERLIKEFSIYMTKDGRISVAGVTSSN
VGYLAHAIHQVTK
Bold/italic: CD8a signal peptide
Plain: anti-ROR-1 scFv (from EP 2884999)
Bold: IgG4 hinge
italic: CD28 transmembrane domain
Underlined: 4-1BB (CD137) co-stimulatory domain
Bold underlined: cytoplasmic signaling domain of human CD3ζ
Bold/underlined/italic: PTA sequence
Italic (3' fragment): GOT2
anti-GPC3 CAR (1)
SEQ ID NO: 89
MALPVTALLLPLALLLHAARPDVVMTQSPLSLPVTPGEPASISCRSSQSLVHSNRNTYLHWYLQKPGQSPQLLIY
KVSNRFSGVPDRESGSGSGTDETLKISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGALDPKTGDTAYSQKFKGRVTLTADKST
STAYMELSSLTSEDTAVYYCTRFYSYTYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPEMRPVQTTQEEDGCSCRFPEEEEGGCEL
RVKESRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
anti-GPC3 CAR (2)
SEQ ID NO: 90
MALPVTALLLPLALLLHAARPDVVMTQSPLSLPVTPGEPASISCRSSQSLVHSNRNTYLHWYLQKPGQSPQLLIY
KVSNRFSGVPDRFSGSGSGTDFTLKISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGALDPKTGDTAYSQKEKGRVTLTADKST
STAYMELSSLTSEDTAVYYCTRFYSYTYWGQGTLVTVSSIEVMYPPPYLDNEKSNGTIIHVKGKHLCPSPLEPGP
SKPFWVLVVVGGVLACYSLLVTVAFIIFWVRSKRSRLLHSDYMNMTPRRPGPTRKHYQPYAPPRDFAAYRSRVKE
SRSADAPAYQQGQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
GOT2
SEQ ID NO: 91
MALLHSGRVLPGIAAAFHPGLAAAASARASSWWTHVEMGPPDPILGVTEAFKRDTNSKKMNLGVGAYRDDNGKPY
VLPSVRKAEAQIAAKNLDKEYLPIGGLAEFCKASAELALGENSEVLKSGREVTVQTISGTGALRIGASFLQRFFK
FSRDVFLPKPTWGNHTPIERDAGMQLQGYRYYDPKTCGFDETGAVEDISKIPEQSVLLLHACAHNPTGVDPRPEQ
WKEIATVVKKRNLFAFFDMAYQGFASGDGDKDAWAVRHFIEQGINVCLCQSYAKNMGLYGERVGAFTMVCKDADE
AKRVESQLKILIRPMYSNPPLNGARIAAAILNTPDLRKQWLQEVKVMADRIIGMRTQLVSNLKKEGSTHNWQHIT
DQIGMFCFTGLKPEQVERLIKEFSIYMTKDGRISVAGVISSNVGYLAHAIHQVTK
GFPT1
SEQ ID NO: 92
MCGIFAYLNYHVPRTRREILETLIKGLQRLEYRGYDSAGVGFDGGNDKDWEANACKIQLIKKKGKVKALDEEVHK
QQDMDLDIEFDVHLGIAHTRWATHGEPSPVNSHPQRSDKNNEFIVIHNGIITNYKDLKKFLESKGYDFESETDTE
TIAKLVKYMYDNRESQDTSFTTLVERVIQQLEGAFALVEKSVHFPGQAVGTRRGSPLLIGVRSEHKLSTDHIPIL
YRTARTQIGSKFTRWGSQGERGKDKKGSCNLSRVDSTTCLFPVEEKAVEYYFASDASAVIEHTNRVIFLEDDDVA
AVVDGRLSIHRIKRTAGDHPGRAVQTLQMELQQIMKGNESSEMQKEIFEQPESVVNTMRGRVNEDDYTVNLGGLK
DHIKEIQRCRRLILIACGTSYHAGVATRQVLEELTELPVMVELASDELDRNTPVERDDVCFELSQSGETADILMG
LRYCKERGALTVGITNTVGSSISRETDCGVHINAGPEIGVASTKAYISQFVSLVMFALMMCDDRISMQERRKEIM
LGLKRLPDLIKEVLSMDDEIQKLATELYHQKSVLIMGRGYHYATCLEGALKIKEITYMHSEGILAGELKHGPLAL
VDKLMPVIMIIMRDHTYAKCQNALQQVVARQGRPVVICDKEDTETIKNTKRTIKVPHSVDCLQGILSVIPLQLLA
FHLAVLRGYDVDEPRNLAKSVIVE
GLUT1
SEQ ID NO: 93
MEPSSKKLTGRLMLAVGGAVLGSLQFGYNTGVINAPQKVIEEFYNQTWVHRYGESILPTTLTTLWSLSVAIFSVG
GMIGSESVGLFVNRFGRRNSMLMMNLLAFVSAVLMGESKLGKSFEMLILGRFIIGVYCGLTTGFVPMYVGEVSPT
ALRGALGTLHQLGIVVGILIAQVEGLDSIMGNKDLWPLLLSIIFIPALLQCIVLPFCPESPRELLINRNEENRAK
SVLKKLRGTADVTHDLQEMKEESRQMMREKKVTILELERSPAYRQPILIAVVLQLSQQLSGINAVFYYSTSIFEK
AGVQQPVYATIGSGIVNTAFTVVSLFVVERAGRRTLHLIGLAGMAGCAILMTIALALLEQLPWMSYLSIVAIFGE
VAFFEVGPGPIPWFIVAELFSQGPRPAAIAVAGESNWTSNFIVGMCFQYVEQLCGPYVFIIFTVLLVLFFIFTYF
KVPETKGRTFDEIASGFRQGGASQSDKTPEELFHPLGADSQV
TIGAR
SEQ ID NO: 94
MARFALTVVRHGETRENKEKIIQGQGVDEPLSETGEKQAAAAGIFLNNVKFTHAFSSDLMRTKQTMHGILERSKF
CKDMTVKYDSRLRERKYGVVEGKALSELRAMAKAAREECPVFTPPGGETLDQVKMRGIDFFEFLCQLILKEADQK
EQFSQGSPSNCLETSLAEIFPLGKNHSSKVNSDSGIPGLAASVLVVSHGAYMRSLEDYFLTDLKCSLPATLSRSE
LMSVTPNTGMSLFIINFEEGREVKPTVQCICMNLQDHLNGLTETR
LDHA
SEQ ID NO: 95
MATLKDQLIYNLLKEEQTPQNKITVVGVGAVGMACAISILMKDLADELALVDVIEDKLKGEMMDLQHGSLELRTP
KIVSGKDYNVTANSKLVIITAGARQQEGESRLNLVQRNVNIFKFIIPNVVKYSPNCKLLIVSNPVDILTYVAWKI
SGFPKNRVIGSGCNLDSARFRYLMGERLGVHPLSCHGWVLGEHGDSSVPVWSGMNVAGVSLKTLHPDLGTDKDKE
QWKEVHKQVVESAYEVIKLKGYTSWAIGLSVADLAESIMKNLRRVHPVSTMIKGLYGIKDDVFLSVPCILGQNGI
SDLVKVTLTSEEEARLKKSADTLWGIQKELQF
PDK1
SEQ ID NO: 96
MRLARLLRGAALAGPGPGLRAAGFSRSFSSDSGSSPASERGVPGQVDFYARFSPSPLSMKQFLDFGSVNACEKTS
EMELRQELPVRLANIMKEISLLPDNLLRTPSVQLVQSWYIQSLQELLDEKDKSAEDAKAIYERPRRIWLQVSSLC
CMACKMIFTDTVIRIRNRHNDVIPTMAQGVIEYKESFGVDPVTSQNVQYELDRFYMSRISIRMLLNQHSLLEGGK
GKGSPSHRKHIGSINPNCNVLEVIKDGYENARRLCDLYYINSPELELEELNAKSPGQPIQVVYVPSHLYHMVFEL
FKNAMRATMEHHANRGVYPPIQVHVTLGNEDLTVKMSDRGGGVPLRKIDRLENYMYSTAPRPRVETSRAVPLAGE
GYGLPISRLYAQYFQGDLKLYSLEGYGTDAVIYIKALSTDSIERLPVYNKAAWKHYNTNHEADDWCVPSREPKDM
TTERSA
ASS1
SEQ ID NO: 97
MSSKGSVVLAYSGGLDTSCILVWLKEQGYDVIAYLANIGQKEDFEEARKKALKLGAKKVFIEDVSREFVEEFIWP
AIQSSALYEDRYLLGISLARPCIARKQVEIAQREGAKYVSHGATGKGNDQVRFELSCYSLAPQIKVIAPWRMPEF
YNREKGRNDLMEYAKQHGIPIPVTPKNPWSMDENLMHISYEAGILENPKNQAPPGLYTKTQDPAKAPNTPDILEI
EFKKGVPVKVTNVKDGTTHQTSLELFMYLNEVAGKHGVGRIDIVENRFIGMKSRGIYETPAGTILYHAHLDIEAF
TMDREVRKIKQGLGLKFAELVYTGFWHSPECEFVRHCIAKSQERVEGKVQVSVLKGQVYILGRESPLSLYNEELV
SMNVQGDYEPTDATGFININSLRLKEYHRLQSKVTAK
PSPH
SEQ ID NO: 98
MVSHSELRKLFYSADAVCFDVDSTVIREEGIDELAKICGVEDAVSEMTRRAMGGAVPFKAALTERLALIQPSREQ
VQRLIAEQPPHLTPGIRELVSRLQERNVQVFLISGGERSIVEHVASKLNIPATNVFANRLKFYENGEYAGFDETQ
PTAESGGKGKVIKLLKEKFHFKKIIMIGDGATDMEACPPADAFIGFGGNVIRQQVKDNAKWYITDFVELLGELEE
CTH
SEQ ID NO: 99
MQEKDASSQGFLPHFQHFATQAIHVGQDPEQWTSRAVVPPISLSTTEKQGAPGQHSGFEYSRSGNPTRNCLEKAV
AALDGAKYCLAFASGLAATVTITHLLKAGDQIICMDDVYGGTNRYFRQVASEFGLKISFVDCSKIKLLEAAITPE
TKLVWIETPTNPTQKVIDIEGCAHIVHKHGDIILVVDNTEMSPYFQRPLALGADISMYSATKYMNGHSDVVMGLV
SVNCESLHNRLRFLQNSLGAVPSPIDCYLCNRGLKTLHVRMEKHFKNGMAVAQFLESNPWVEKVIYPGLPSHPQH
ELVKRQCTGCTGMVTFYIKGTLQHAEIFLKNLKLETLAESLGGFESLAELPAIMTHASVLKNDRDVLGISDTLIR
LSVGLEDEEDLLEDLDQALKAAHPPSGSHS
```

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doylc, J. B. Griffiths, and D. G. Ncwell, cds. 1993-8) J. Wilcy and Sons; Methods in Enzymology (Academic Press, Inc.); Handbook of Experimental Immunology (D. M. Wcir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984»; Animal Cell Culture (R. I. Freshney, ed. (1986); Immobilized Cells and Enzymes (IRL Press, (1986); and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are intended only to illustrate methods and embodiments in accordance with the invention, and as such should not be construed as imposing limitations upon the claims.

### Example 1: Production of Retroviral Particles and Viral Titer Determination

15×10⁶ low passage HEK293 cells were plated in 15 cm coated tissue culture plates using DMEM (+10% FBS) media so that the following day they were approximately 80% confluent. The next day, 1.5 ml of transfection mixture containing 10 μg of GAG/Pol, 6.6 mg of GALV helper plasmids, 20 μg of transfer plasmid and Opti-MEM (Cat #31985062; Thermo Fisher:) were mixed with 1.5 ml of Opti-MEM, and 75 μl of PEIPro transfection reagent (PolyPlus: 115-010) were added to the plates. Media was changed after 6 h with fresh DMEM (+10% FBS). Viral supernatants were harvested at 24 h and clarified through a 0.45 micron filter and stored at −80° C.

### Example 2: PBMC Initiation and Viral Transduction

Approximately 100×10⁶ PBMCs per donor were initiated by stimulation with anti-CD3 (Cat #130-093-387, Miltenyi), anti-CD28 (Cat #130-093-375; Miltenyi), and IL-2 (100 UI/ml; Cat #1020-500; CellGenix) in X Vivo 15 (Cat #BEBP04-744Q; Lonza) in 100 ml on Day 0 in T175 flaks. On Day 2, activated PBMC were harvested and 1 ml containing 1×10⁶ cells were plated in a 24 well plate. For transduction, 1 ml of viral supernatant was added to each well. Plates were centrifuged 1200 g for 45 min and allowed to rest overnight in an incubator at 37° C. One day after transduction, cells were transferred to a GREX vessel (Cat #80192M; Wilson Wolf) filled with X-VIVO+100 IU/ml IL-2. The cells were maintained for an additional week with supplementation of IL-2 every 48 h. On Day 10, CAR T or BOXR CAR T cells were harvested and frozen until further analysis.

The transduced immune cells were assessed for the transgene expression by immunoblotting. In brief, the transduced cells (e.g., T cells or NK-92) were harvested by centrifuging at 1500 rpm for 5 min at RT. The supernatant was removed, and the cell pellet washed twice in 1×PBS before flash freezing in liquid nitrogen and stored at −80° C. until further use. Cell pellets were subsequently lysed in 200 μl of SDS Lysis buffer (Cat #NP0008; Novex) containing 1× HALT Protease Inhibitor Cocktail (Cat #78430; Thermo Fisher) followed by sonication. The suspension was centrifuged at 15,000 rpm for 15 min at RT and the supernatant containing total protein was collected. The total protein concentration was measured using Pierce 660 nm Protein Assay (Cat #1861426; Thermo Scientific) followed by immunoblotting. 10 μg total protein was loaded in each lane of a Novex™ 4 to 12% Tris-Glycine Plus, 1.0 mm, 20-well Midi Protein Gel (Invitrogen), transferred onto PVDF membrane using Transblot Turbo (Biorad) and blocked for 1 h at RT using LICOR Blocking buffer. The membrane was probed for transgenes (e.g., GOT2, TIGAR) using mouse α-Actin (3700S, CST; dilution 1:2000), Rabbit α-TIGAR (14751S CST; dilution 1:1000) and Rabbit α-Got2 (NBP232241, Novus; dilution 1:2000) antibodies overnight (in 0.1% Tween 20+LICOR Blocking buffer) at 4° C. The following day, membranes are washed thrice with 1×TBS containing 0.1% Tween20 detergent (w/v) for 5 min each. Membranes are subsequently incubated with standard rabbit or mouse secondary antibodies (LICOR; dilution 1:10,000) for 1 h. The membranes were washed thrice with 1×TBS containing 0.1% Tween20 detergent (w/v) for 5 min each. Immunoblots are imaged using a CLX imager (LICOR) and processed in the Image Studio Software (v5.2; LICOR).

### Example 3: Flow Cytometry and Analysis (FACS)

Flow cytometry experiments were performed on an Attune flow cytometer (Thermo Fisher). Cells were collected and washed in PBS. Cells were stained in a 1:1000 dilution of live dead dye in PBS (Zombie Aqua; Cat #423102; Biolegend) for 10 min at room temperature in the dark. Cells were washed in FACS buffer (PBS+2% FBS). Cells were stained for CAR with ROR1-Fc-biotin protein, streptavidin-AF647 and cells surface markers (CD3, CD4, CD8, CD45RA, CCR7), see Table 7 below. For Ki67 staining, cells were further processed and washed in PBS+2% FBS then fixed and permeabilized for 30 minutes at 4° C. following manufacturer's protocol (Cat #00-5523-00; ebiosciences).

Following fixation, cells were washed with 1× permeabilization buffer (Cat #00-5523-00; ebiosciences) twice. Ki67 antibody was diluted to 1:50 in 1× permeabilization buffer and added to cells, mixed well, incubated for 30 minutes at 4° C. After incubation, cells were washed and resuspended in FACS buffer. Data was acquired on an Attune NXT. Data analysis included selecting for single living single cells. CAR expression was determined in comparison to an untransduced control. Cell populations were gated as described in figure legends. Data were analyzed with FlowJo version 10.7.1 software (Tree Star Inc., Ashland, OR, USA).

TABLE 7
Detecting Agents for Cell Surface Markers

| Marker | Fluorophore | Catalog # | | Clone |
|---|---|---|---|---|
| CD45RA | BV605 | 3041334 | (Biolegend) | HI100 |
| CD4 | BV711 | 300558 | (Biolegend) | RPA-T4 |
| CCR7 | AF488 | 353206 | (Biolegend) | G043H7 |
| Ki67 | PE | 350504 | (Biolegend) | Ki67 |
| CD8 | AF700 | 344729 | (Biolegend) | SKI |
| CD3 | APC/Cy7 | 300426 | (Biolegend) | UCHT1 |
| Streptavidin-647 | AF647 | 405237 | (Biolegend) | N/A |
| ROR1-Fc-biotin | N/A | RO1-H82F4 | (AcroBiosystems) | N/A |

### Example 4: Proliferation of Activated T Cell Products

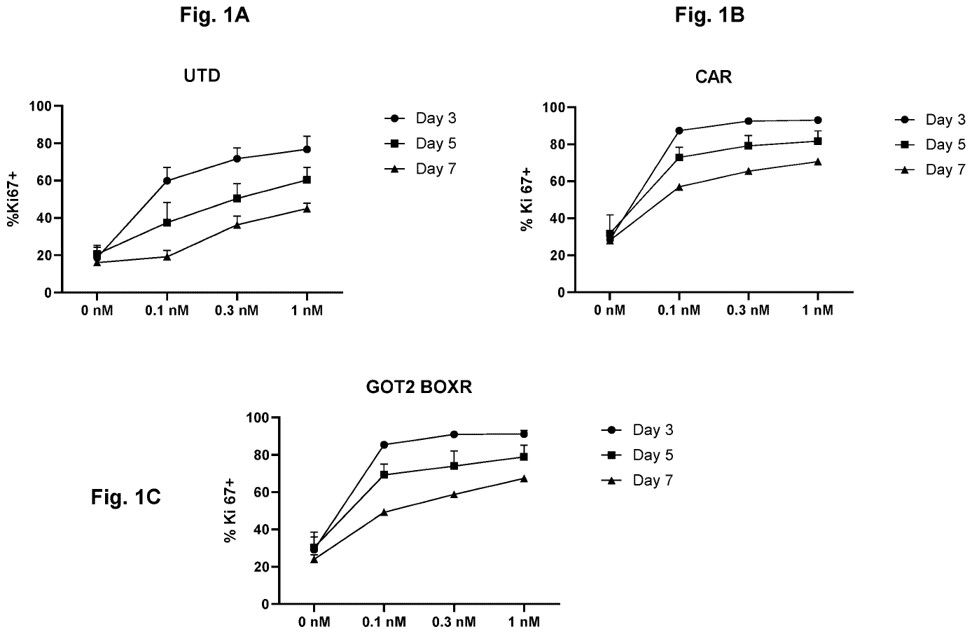

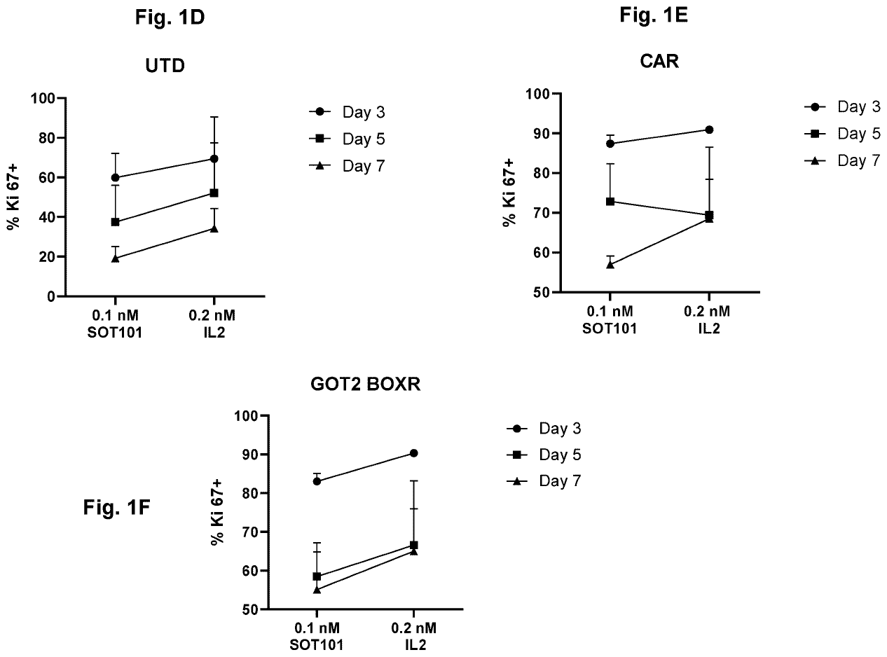

In order to determine a suitable dose of SOT101 (SEQ ID NO: 79) and IL-2, untransduced T cells, CAR T cells expressing an anti-ROR-1 CAR or BOXR CAR T cells co-expressing exogenous GOT2 and an anti-ROR-1 CAR were seeded in a 96-well U bottom plate and treated with SOT101 at concentrations from 0 to 1 nM, or with 0.2 mM IL-2. At day 3, 5, or 7, cells were stained for cell markers, intracellularly stained for the proliferation marker Ki67 and analyzed by FACS. Maximum proliferation (Ki67⁺) of CAR-expressing T cells was achieved after 3 days of treatment with 0.1 nM SOT101 (FIGS. 1A to 1C). There was a trend that 0.1 nM SOT101 was inferior to 0.2 mM IL-2 (FIGS. 1D to 1F).

### Example 5: Analysis of T Cell Memory Subsets after SOT101 Treatment

CAR-expressing T cells from Example 4 above stimulated with 0.1 mM SOT1010 or 0.2 mM IL-2 were further analyzed by FACS to group them into the subtypes of memory T cell being (i) CD45RA⁺, CCR7⁺ stem cell like T cell (T_{scm}); (ii) CD45RA, CCR7⁺ naïve central memory T cell (T_{CM}), (iii) CD45RA⁻, CCR7⁻ effector memory T cell (T_{EM}), or (iv) CD45RA⁺, CCR7⁻ effector memory cells re-expressing CD45RA (TcmRA). T cell differentiation goes from T_{scm} to T_{CM} to T_{EM} to T_{emRA}. T cell memory subsets were comparable for CAR T cells expressing only the anti-ROR-1 CAR or the anti-ROR-1 CAR and GOT2.

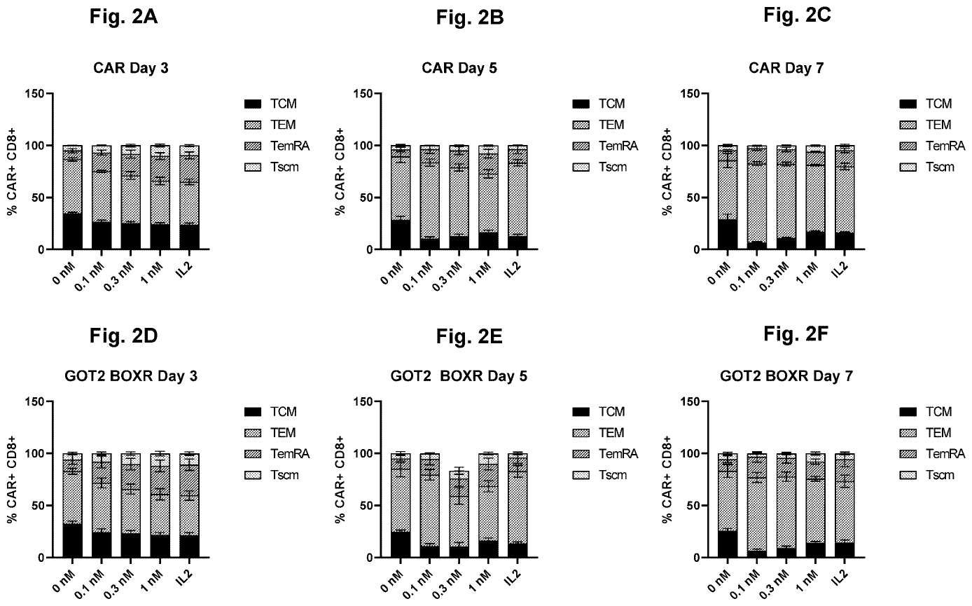

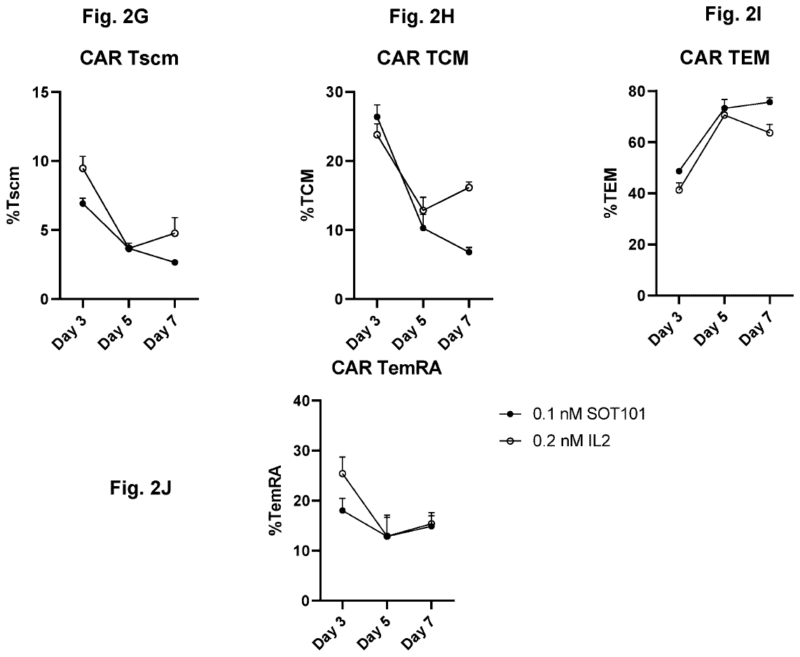

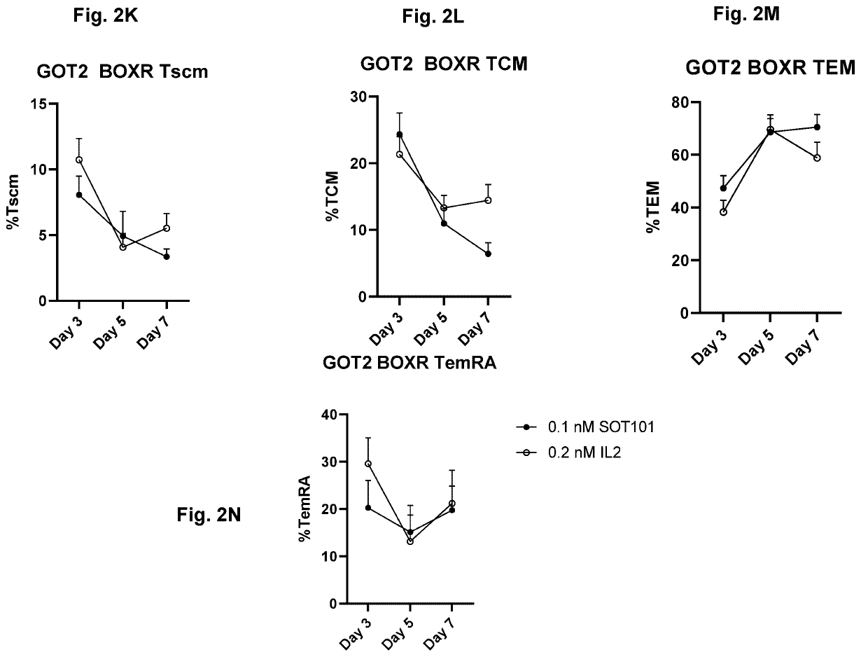

Comparing increasing doses of SOT101, a slight dose-dependent increase of T_{CM} was observed at day 5 and day 7 (although still lower than in absence of SOT101). Overall, a slight shift in T cell memory populations was observed with increasing SOT101 doses. (FIGS. 2A-2C and FIGS. 2D-2F)

From day 3 to day 7, we observed slight shifts in T cell memory populations towards T_{EM}, with corresponding decreases in T_{scm} and T_{CM}, resulting in a more differentiated (T_{EM}) phenotype.

Comparing SOT101 and IL-2, at day 7 this trend was more pronounced for SOT101 compared to IL-2 treatment. Further, at day 3, T_{scm} and T_{emRA} populations were reduced with SOT101 compared to IL-2 and we observed a reduction for both T_{scm} and T_{CM} and an increase in T_{EM} at day 7. In summary, SOT101 treatment resulted in more differentiated (T_{EM}) phenotype compared to IL-2 treatment. (FIGS. 2G-2J and FIGS. 2K-2N)

### Example 6: In Vitro Cytotoxicity of CAR T/BOXR CAR T Cells Upon SOT101 or IL-2 Pre-Treatment

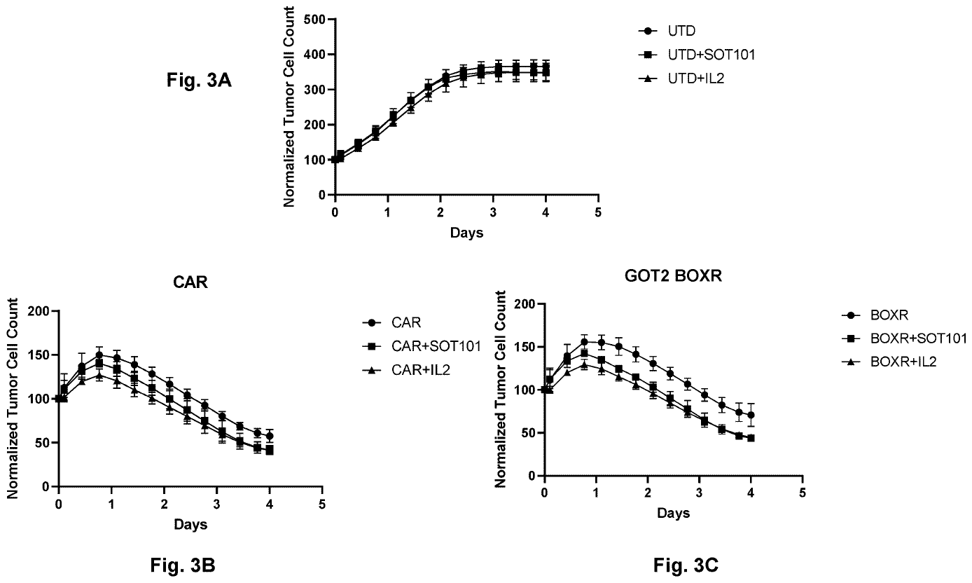

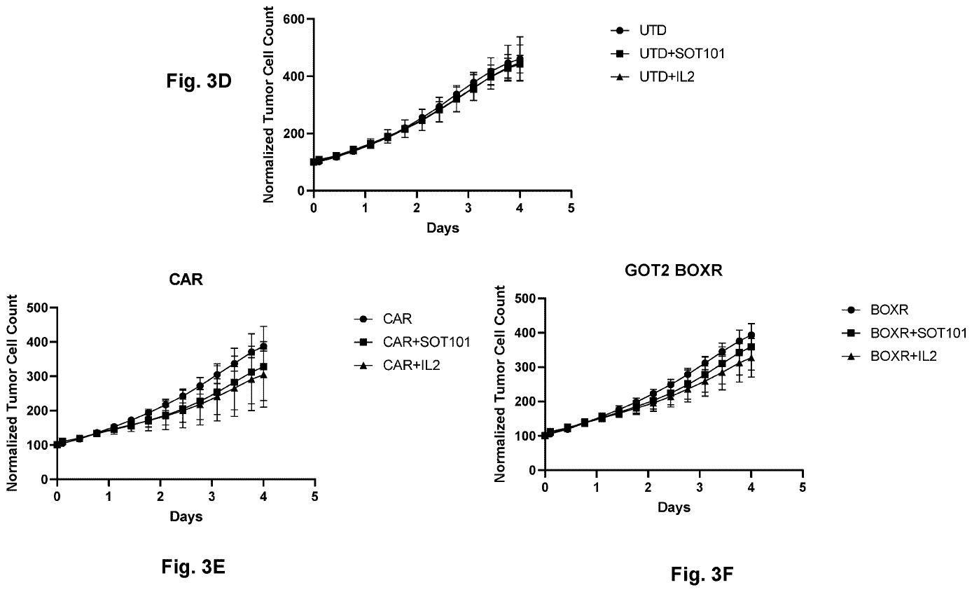

Untreated T cells (UTD), anti-ROR-1 CAR T cells (CAR) or GOT2 expressing anti-ROR-1 CAR T cells (GOT2 BOXR) were treated with 0.1 nM SOT101 or IL-2 for 3 days then applied to A549 ROR-1⁺ tumor cells or Hep3b ROR-1-tumor cells and counts of tumor cells were determined for four days. Whereas tumor cells amplified while being incubated with untreated T cells, an increase in T cell cytotoxicity in vitro was observed for both CAR T cells and GOT2 BOXR T cells with SOT101 or IL-2 treatment for ROR-1 expressing tumor A549 tumor cells (FIGS. 3A-3C). This increase was antigen-specific, as only a minor change was observed for the ROR-1 negative Hep3b control tumor cells (FIGS. 3D-3F).

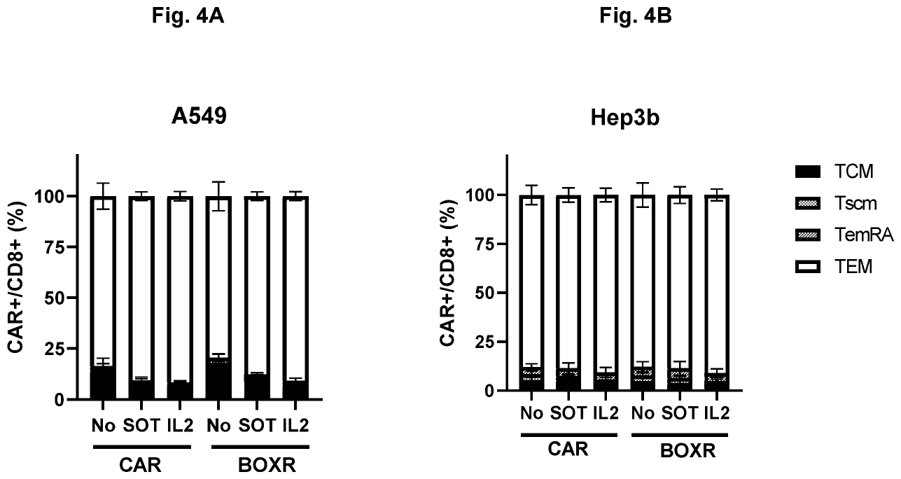

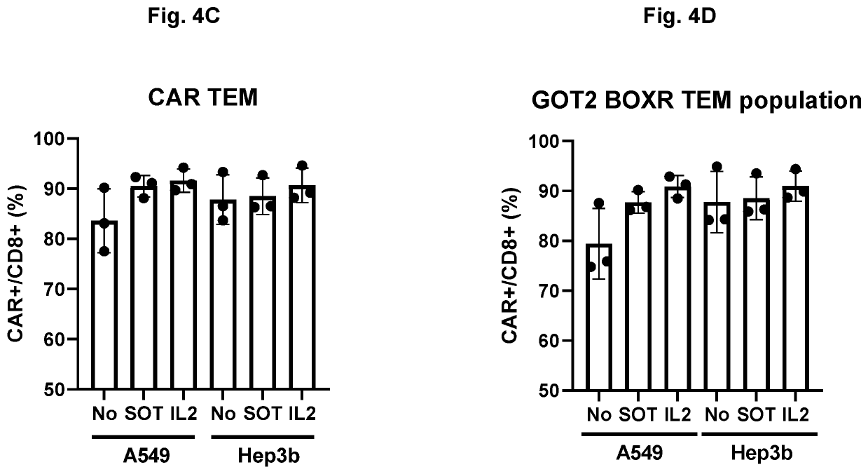

After 3 days of co-culturing with ROR-1⁺ A549 tumor cells or ROR-1-Hep3b tumor cells, CAR or GOT2 BOXR T cells were analyzed on day 4 by FACS for T cell differentiation markers. When cultured with antigen positive tumor cells, cytokine treatment with both SOT101 and IL-2 resulted in an increase in the T_{EM} population (CD45RA⁻, CCR7⁻) for both CAR T cells (FIGS. 4A and 4C) and GOT2 BOXR T cells (FIGS. 4A and 4D), whereas no significant change was observed for antigen negative tumor cells (FIG. 4B).

### Example 7: In Vivo Efficacy Using a Caki-1 (Human Renal Cell Carcinoma) Xenograft Mouse Model

Female, 5-6 week-old NOD scid gamma (NSG) mice were implanted with 5 million Caki-1 cells suspended in 50% RPMI 1640: matrigel via s.c. injection into the right flank. When tumors reached 100-120 mm³, 5 mice per group were dosed with 3 million anti-ROR-1 CAR⁺ T cells via i.v. tail vein injection (Day 0). SOT101 was administered s.c. at doses of 15 μg/mouse on days 7, 8, 14 and 15. Tumor growth and body weight were monitored 2 times per week. Tumor volume was measured using calipers and calculated using the formula (L×W×H)/2 mm³.

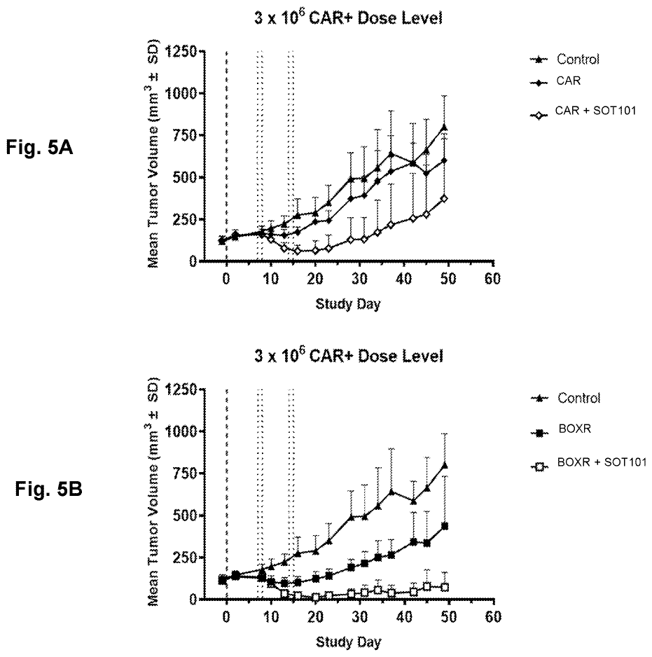

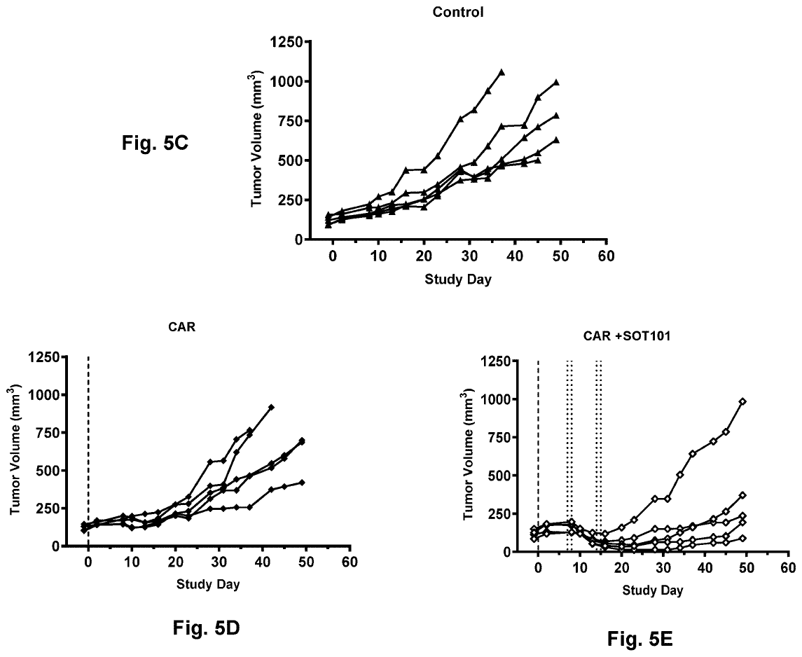

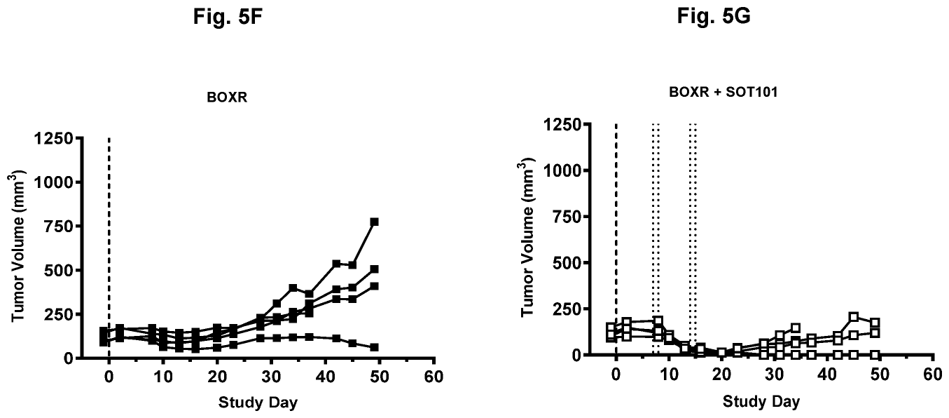

For the CAKI-1 xenograft model, anti-ROR-1 CAR⁺ T cells had a minor effect on tumor growth (measured as mean tumor volume), whereas the addition of SOT101 substantially reduced the tumor volume (FIG. 5A). In the same experiment, anti-ROR-1 GOT2 expressing BOXR T cells showed a stronger anti-tumor effect compared to the CAR⁺ T cells. This antitumor effect was strongly enlarged by the additional treatment with SOT101 (FIG. 5B). This synergistic effect can be further observed looking at the individual mouse data for the groups in FIGS. 5C to 5G. Whereas the CAR⁺ T cells alone only slowed-down tumor growth (D), the addition of SOT101 starting at day 7 lead to decrease in tumor volume shortly after day 7 with a following slow tumor growth except for one mouse. CAR⁺ T cells co-expressing GOT2 showed a slightly higher antitumor response with one remission (FIG. 5F), whereas, again, the additional treatment with SOT101 starting at day 7 lead to remissions in all animals followed by a continuing inhibition of tumor growth until the study was terminated at day 50 (FIG. 5G).

There was a slight overall body weight loss (BWL) within the groups, which was neither dose dependent nor additive, and no mice reached euthanasia criteria of −20% BWL. As such, it is believed that this observation is due to overhandling of mice rather than to drug toxicity. Therefore, the observed slight overall BWL was interpreted as a result from stress due to handling of mice every day for weighing and multiple dosing.

Similar results were obtained when dosed with 1.5 million anti-ROR-1 CAR⁺ T cells in combination with 15 μg of SOT101/mouse as well as with a suboptimal dosing schedule with 5 million anti-ROR-1 CAR⁺ T cells and 5 or 15 μg of SOT101/mouse at days 7 and 14. In summary, the combination of the CAR T cell therapy or the BOXR CAR T cell therapy, here expressing the GOT2 metabolism modulating polypeptide, with an IL-15/IL-15Rα conjugate, here the IL-15/IL-15Rα sushi+ fusion protein SOT101, was well tolerated and lead to likely additive to even synergistic treatment improvements in the CAKI-1 xenograft model in vivo. SOT101 clearly improved the efficacy of CAR-expressing T cells, and this effect was even stronger when the BOXR T cells co-expressed an exogenous copy of a gene optimizing the metabolism of the T cell, here the GOT2 gene.

### Example 8: Pharmacokinetics

Blood from retro-orbital bleeds of mice from Example 7 above was collected on study days 7, 14, 21 and 28, and 20 μl of blood were frozen with 80 μl of Bambanker (Cat #BB01; Bulldog bio) and stored at −80° C. Samples were thawed, washed with PBS and lysed with 150 μl of FACS lysing solution (Cat #349202; BD Biosciences) for 5 min. Cells were washed with PBS and stained with 1:1000 dilution of Live/Dead in PBS for 10 min. Cells were washed in FACS buffer with FC block (1:200, mouse and human, Cat #422302, 101320; Trustain FcX), and stained with cell surface markers (mouse CD45-FITC: 103108, human CD3-AF647). Data were acquired on Attune (Thermo Fisher) acquiring a set volume of sample. CD3⁺ cell counts were calculated from acquired CD3⁺ cell counts and normalized for the amount of sample acquired, and starting blood volume.

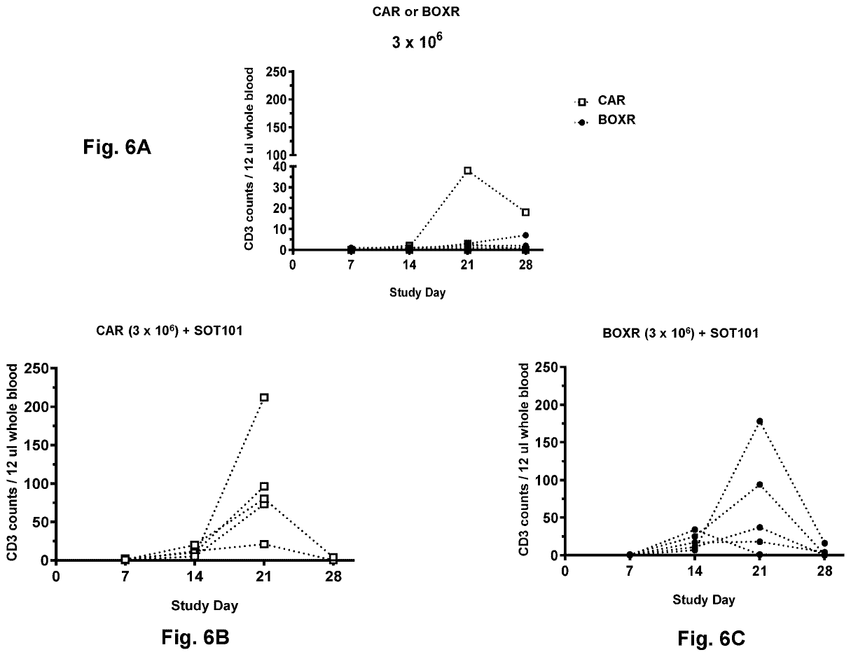

In absence of SOT101 treatment, only one mouse showed an increase in CD3⁺ cells within the 20 days of monitoring (FIG. 6A). Treatment with SOT101 in combination with CAR T cells (FIG. 6B) or BOXR T cells (FIG. 6C) resulted in an improved expansion of CD3⁺ cells in most of the animals after day 14, which dropped again to close to 0 at day 28.

### Example 9: In Vivo Efficacy Using NCI-H1975 (Human Non-Small Cell Lung Carcinoma) Xenograft Mouse Model

Female, 5-6 week-old NOD scid gamma (NSG) mice were implanted with 5 million NCI-H1975 tumor cells suspended in 50% RPMI 1640: matrigel via s.c. injection into the right flank. When tumors reached 100-120 mm³, 5 mice per group were dosed with 3 million anti-ROR-1 CAR⁺ T cells via i.v. tail vein injection (Day 0). SOT101 was administered s.c. at doses of 15 μg/mouse on days 7, 8, 14 and 15. Tumor growth and body weight were monitored 2 times per week. Tumor volume was measured using calipers and calculated using the formula (L×W×H)/2 mm³.

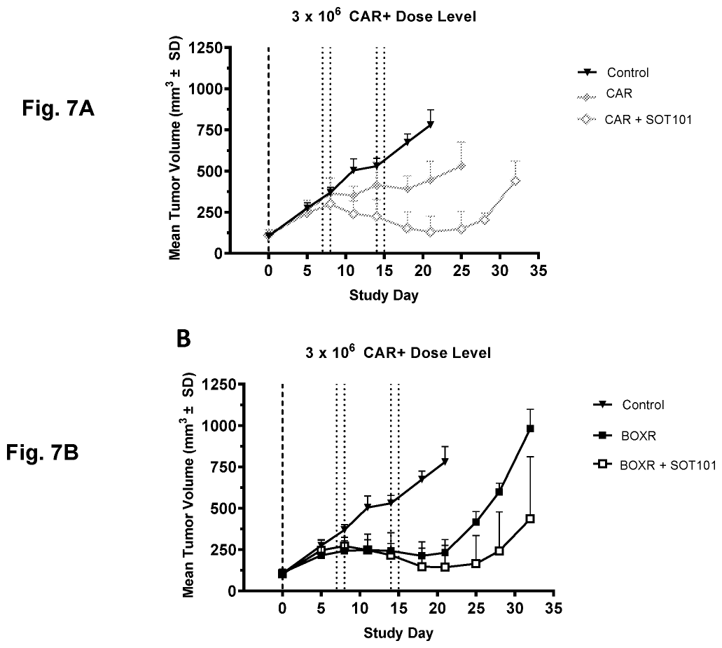

Similar to the CAKI-1 xenograft model, also in the NCI-H1975 xenograft model the anti-ROR-1 CAR⁺ T cells had a minor effect on tumor growth (measured as mean tumor volume), whereas the addition of SOT101 substantially reduced the tumor volume (FIG. 7A). In the same experiment, anti-ROR-1 GOT2 expressing BOXR T cells again showed a stronger anti-tumor effect compared to the CAR⁺ T cells and this antitumor effect was enlarged by the additional treatment with SOT101 (FIG. 7B). However, in this model no difference was observed between the groups CAR T cells in combination with SOT101 and GOT2 BOXR T cells with SOT101.

### Example 10: Impact of Expressing Metabolism Modulating Polypeptide on T Cell Function

Gamma-retrovirus encoding the test antigen-targeting CAR polypeptide expression construct may be generated via recombinant technology and for use to infect primary human T-cells to generate cells expressing the test antigen-targeting CAR polypeptide on their cell surface. Additionally, gamma-retroviruses encoding the test antigen-targeting CAR polypeptide and a test metabolism modulating polypeptide (e.g., GOT2, GLUT1, TIGAR or other metabolism modulating polypeptides of interest) are to be generated via recombinant technology and used to infect primary human T-cells to generate cells that express the test antigen-targeting CAR polypeptide and the test metabolism modulating polypeptide. It is preferred that both transgenes are encoded on one vector to ensure that cells do not get transfected with only one vector. Accordingly, transfected cells expressing the CAR, which can easily be determined, e.g., by FACS analysis, also contain the metabolism modulating polypeptide.

In the constructs encoding both the CAR polypeptide and the test metabolism modulating polypeptide, the two polypeptides may be separated by a P2A ribosomal skip sequence. Alternatively, an IRES sequence or a second expression cassette/second promoter may be used to express two transgenes from one vector. A six-day flow-based proliferation assay can then be used to test the functionality of the test antigen-targeting CAR expressing cells compared to control. Specifically, 200,000 untransduced mock-cells, T-cells expressing the test antigen-targeting CAR polypeptide, or T-cells expressing both the test antigen-targeting CAR polypeptide and the test metabolism modulating polypeptide are incubated together at a ratio of 4:1 (effector CAR-expressing T cells to target cells) with 50,000 suitable, antigen-expressing tumor cells of an established tumor cell line. The co-culture is incubated at typical conditions for the tumor cell line, e.g., 37° C. in a 5% CO₂, for six days in the presence of 1.25 mM glucose (tumor-relevant) or 10 mM glucose (approximate peripheral blood levels). At the end of six days, co-cultures are harvested and stained with an anti-CD3 antibody.

The analogous assay may be conducted by replacing the CAR polypeptide by the respective chimeric receptor polypeptide, e.g., by a TCR or an ACTR polypeptide, and ensuring that the matching antigen is expressed by the tumor cell line and/or that, in case of an ACTR polypeptide, the respective anti-tumor antibody is added to the assay, to allow for specific binding of the immune cell to tumor cell. In case of immune cells without a chimeric receptor polypeptide, e.g., NK cells, γδ T cells or TILs, the assay is to be conducted by only comparing respective untransduced immune cells with immune cells transduced with the gene encoding the test metabolism modulating polypeptide.

For measuring T cells, the number of CD3⁺ cells is evaluated by flow cytometry as a measure of T cell proliferation. T cells expressing the test metabolism modulating polypeptides in addition to the CAR polypeptide relative to T cells expressing the CAR construct alone are measured to determine the induction of proliferation of the immune cell population. This enhanced proliferation also occurs at tumor-relevant low glucose concentrations. These experiments demonstrate that expressing Krebs cycle modulating polypeptides in T cells has a positive impact on CAR-T cell proliferation activity.

For measuring NK cells, CD3⁺ T cells are to be excluded by FACS sorting. Then, CD56 and CD16 are used to define the main subsets of NK cell, CD56^{bright}CD16^{lo/-} and CD56^{dim}CD16⁺, often simplified as CD56^{bright} and CD56^{dim}, respectively. γδ T cells can be measured through FACS by gating for CD3⁺ and γδ-TCR⁺ gating.

### Example 11: Expression of GLUT1, GOT2, and TIGAR Elevated Glucose Uptake and Lactate Production

Healthy donor PBMCs are stimulated with anti-CD3 and anti-CD28 until day 2 followed by transduction with transgene packaged into a lentiviral vector. Examples for metabolism modulating polypeptides are GLUT1 (SEQ ID NO: 93), GOT2 (SEQ ID NO: 91), or TIGAR (SEQ ID NO: 75). The transduced cells are supplemented with fresh IL-2 each day until day 10. 10,000 cells/well (384-well plate) are resuspended in PBS and assayed for glucose uptake. The luminescence read-out is evaluated as a fold change for each transgene and compared to null (non-transduced control; baseline as fold change 1) T cells under the same condition. Cells showing elevated levels of glucose uptake are indicative of higher metabolic activity.

Further, healthy donor PBMCs are stimulated with anti-CD3 and anti-CD28 until day 2 followed by transduction with transgene (described above) packaged into lentiviral vectors. The transduced cells are supplemented with fresh IL-2 each day until day 9. On day 9, a subset of T cells is stimulated with PMA and Ionomycin for 24 h. 10,000 harvested cells/well (384-well plate) are resuspended in RPMI without FBS and incubated at 37° C. for 2 h to remove residual lactate from the media and assayed for lactate production. The luminescence read-out is evaluated as a fold change for each transgene compared to null (non-transduced control; baseline as fold change 1) T cells under the same condition. Stimulated cells showing elevated levels of lactate production are indicative of higher metabolic adaptability in nutrient deficient environments.

Previous data show that T cells transduced with GLUT1, GOT2, or TIGAR showed enhanced metabolic activity and adaptability in nutrient deficient environment. This indicates that therapeutic T cells (e.g., T cells expressing an ACTR or CAR polypeptide as disclosed herein) co-expressing for e.g., GLUT1, GOT2, or TIGAR would be better adapted to tumor microenvironment (which could be deficient in nutrient) and exhibit better therapeutic activity as compared with counterpart T cells that are not transduced with the transgene (e.g., GLUT1, GOT2, or TIGAR). See WO2020/010110, WO2020/037066 and WO 2023/049933 the relevant disclosures of each of which are incorporated by reference for the subject matter and purpose referenced herein.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

It should also be understood that, unless clearly indicated to the contrary, in any methods claimed herein that include more than one step or act, the order of the steps or acts of the method is not necessarily limited to the order in which the steps or acts of the method are recited.

### REFERENCES

Arenas-Ramirez, N., et al. (2016). Sci Transl Med 8(367): 367ra166.
Bacac, M., et al. (2017). Cancer Research 77(13 Supplement): 1594.
Bacac, M., et al. (2016). Clin Cancer Res 22(13): 3286-3297.
Bernett, M. J., et al. (2017). Proceedings of the American Association for Cancer Research 58:408.
Bettini, M. L., et al. (2017). J Immunol 199(5): 1555-1560.
Brentjens, R. J., et al. (2013). Sci Transl Med 5(177): 177ra138.
Brentjens, R. J., et al. (2003). Nat Med 9(3): 279-286.
Brentjens, R. J., et al. (2011). Blood 118(18): 4817-4828.
Caffaro, C. E., et al. (2019). Discovery of pharmacologically differentiated Interleukin 15 (IL-15) agonists employing a synthetic biology platform. SITC 2019. National Harbor, Maryland.
Cao, Y., et al. (2016). Angew Chem Int Ed Engl 55(26): 7520-7524.
Cartellieri, M., et al. (2016). Blood Cancer J 6(8):e458.
Castro, I., et al. (2011). J Immunol 187(10): 5170-5182.
Charych, D., et al. (2017). PLOS One 12(7):e0179431.
Charych, D. H., et al. (2016). Clinical Cancer Research 22(3): 680.
Chenoweth, M. J., et al. (2012). J Immunol 188(9): 4149-4157.
Cho, J. H., et al. (2018). Cell 173(6): 1426-1438.e1411.
Conlon, K. C., et al. (2019). J Interferon Cytokine Res 39(1): 6-21.
Cooper, L. J., et al. (2003). Blood 101(4): 1637-1644.
Du, N., et al. (2021). Cancers 13(16): 4129.
Edgar, R. C. (2004). Nucleic Acids Res 32(5): 1792-1797.
Eshhar, Z., et al. (1993). Proc Natl Acad Sci USA 90(2): 720-724.
Fehniger, T. A. and M. A. Caligiuri (2001). Int Rev Immunol 20 (3-4): 503-534.
Gearing, A. J. and R. Thorpe (1988). J Immunol Methods 114(1-2): 3-9.
Geiger, T. L., et al. (1999). The Journal of Immunology 162(10): 5931-5939.
Ghasemi, R., et al. (2016). Nat Commun 7:12878.
Giron-Michel, J., et al. (2005). Blood 106(7): 2302-2310.
Gong, Y., et al. (2021). J Hematol Oncol 14(1): 73.
Goujon, M., et al. (2010). Nucleic Acids Res 38 (Web Server issue): W695-699.
Gubin, M. M., et al. (2015). J Clin Invest 125(9): 3413-3421.
Han, K. P., et al. (2011). Cytokine 56(3): 804-810.
Harada, H., et al. (2002). Jpn J Cancer Res 93(3): 313-319.
Harada, H., et al. (2004). Exp Hematol 32(7): 614-621.
Heaton, K. M., et al. (1993). Cancer Res 53(11): 2597-2602.
Hori, T., et al. (1987). Blood 70(4): 1069-1072.
Hu, P., et al. (2003). Blood 101(12): 4853-4861.
Hu, Q., et al. (2018). Sci Rep 8(1): 7675.
Imai, C., et al. (2004). Leukemia 18(4): 676-684.
Jayaraman, J., et al. (2020). EBioMedicine 58:102931.
Joseph, I. B., et al. (2019). Proceedings of the American Association for Cancer Research 60:838.
Kim, J. H., et al. (2011). PLOS One 6(4): e18556.
Kim, M. S., et al. (2015). J Am Chem Soc 137(8): 2832-2835.
Klein, C. (2014). Journal for Immunotherapy of Cancer 2(Suppl 2): 18-18.
Klein, C., et al. (2013). Cancer Research 73(1 Supplement): PR8.
Klein, C., et al. (2017). Oncoimmunology 6(3): e1277306.
Klein, E., et al. (1976). Int J Cancer 18(4): 421-431.
Knudson, K. M., et al. (2020). Expert Opin Biol Ther 20(7): 705-709.
Kochenderfer, J. N., et al. (2012). Blood 119(12): 2709-2720.
Kudo, K., et al. (2014). Cancer Res 74(1): 93-103.
Kuo, M. L., et al. (1993). Blood 82(3): 845-852.
Kurowska, M., et al. (2002). J Immunol 169(4): 1760-1767.
Lazear, E., et al. (2017). Oncoimmunology 6(2): e1265721.
Li, Y., et al. (2018). Cell Stem Cell 23(2): 181-192.e185.
Linnemann, C., et al. (2015). Nat Med 21(1): 81-85.
Liu, B., et al. (2018). Cytokine 107:105-112.
Liu, E., et al. (2018). Leukemia 32(2): 520-531.
Lohmueller, J. J., et al. (2017). Oncoimmunology 7(1): e1368604.
Lopes, J. E., et al. (2020). J Immunother Cancer 8(1).
Lozzio, C. B. and B. B. Lozzio (1975). Blood 45(3): 321-334.
Ma, J. S., et al. (2016). Proc Natl Acad Sci USA 113(4): E450-458.
Mann, R., et al. (1983). Cell 33(1): 153-159.
Margolin, K., et al. (2018). Clin Cancer Res 24(22): 5552-5561.
Markowitz, D., et al. (1988). J Virol 62(4): 1120-1124.
Mendes, B. B., et al. (2022). Nature Reviews Methods Primers 2(1): 24.
Miyazaki, T., et al. (2018). Blood 132(Suppl 1): 2952-2952.
Morgan, M. A., et al. (2020). Front Immunol 11:1965.
Myers, J. A. and J. S. Miller (2021). Nature Reviews Clinical Oncology 18(2): 85-100.
Needleman, S. B. and C. D. Wunsch (1970). J Mol Biol 48(3): 443-453.
Nussbaumer, O. and M. Koslowski (2019). Immunooncol Technol 1:3-10.
Park, J. H. and H. K. Lee (2021). Exp Mol Med 53(3): 318-327.
Pearson, W. R. and D. J. Lipman (1988). Proc Natl Acad Sci USA 85(8): 2444-2448.
Perdreau, H., et al. (2010). Eur Cytokine Netw 21(4): 297-307.
Porter, D. L., et al. (2011). New England Journal of Medicine 365(8): 725-733.
Pule, M. A., et al. (2008). Nat Med 14(11): 1264-1270.
Rabinovich, P. M., et al. (2006). Human Gene Therapy 17(10): 1027-1035.
Rhode, P. R., et al. (2016). Cancer Immunol Res 4(1): 49-60.
Ring, A. M., et al. (2012). Nat Immunol 13(12): 1187-1195.
Robinson, T. O. and K. S. Schluns (2017). Immunol Lett 190:159-168.
Rosen, D. B., et al. (2022). J Immunother Cancer 10(7).
Schmidt, P., et al. (2020). Front Immunol 11:611163.
Shafer, P., et al. (2022). Front Immunol 13:835762.
Shanafelt, A. B., et al. (2000). Nat Biotechnol 18(11): 1197-1202.
Sievers, N. M., et al. (2020). Int J Mol Sci 21(10).
Silva, D.-A., et al. (2019). Nature 565(7738): 186-191.
Smith, T. F. and M. S. Waterman (1981). Advances in Applied Mathematics 2(4): 482-489.
Soman, G., et al. (2009). J Immunol Methods 348(1-2): 83-94.
Steel, J. C., et al. (2012). Trends Pharmacol Sci 33(1): 35-41.
Sterner, R. C. and R. M. Sterner (2021). Blood Cancer Journal 11(4): 69.
Tamada, K., et al. (2012). Clin Cancer Res 18(23): 6436-6445.
Thaysen-Andersen, M., et al. (2016). Glycoconj J 33(3): 417-433.
Till, B. G., et al. (2012). Blood 119(17): 3940-3950.
Toutain, P. L. and A. Bousquet-Melou (2004). J Vet Pharmacol Ther 27(6): 427-439.
Urbanska, K., et al. (2012). Cancer Res 72(7): 1844-1852.
Wadhwa, M., et al. (2013). Journal of Immunological Methods 397(1): 1-7.
Wang, S., et al. (2021). BMC Medicine 19(1): 140.
Wang, W., et al. (2020). Cancer Lett 472:175-180.
Wei, X., et al. (2001). J Immunol 167(1): 277-282.
Wieczorek, M., et al. (2017). Front Immunol 8:292.
Wrona, E., et al. (2021). Int J Mol Sci 22(11).
Xie, G., et al. (2020). EBioMedicine 59:102975.
Ying, Z., et al. (2019). Nat Med 25(6): 947-953.

1. A method for treatment of cancer in a subject, comprising administering an immune cells population to the subject in need thereof in combination with an Interleukin-2/Interleukin-15 receptor beta gamma (IL-2/IL-15Rβγ) agonist,
wherein the immune cell population comprises immune cells, which are genetically engineered to express or overexpress a polypeptide that:
(a) improves glucose uptake activity;
(b) modulates the Krebs cycle;
(c) enhances lactate concentration; and/or
(d) diverts or redirects glucose metabolites out of a glycolysis pathway.

2. The method of claim 1, wherein the polypeptide that
(a) improves glucose uptake activity is a glucose importation polypeptide, preferably wherein the glucose importation polypeptide is a glucose transporter (GLUT) or a sodium-glucose cotransporter (SGLT), preferably wherein the glucose importation polypeptide comprises of GLUT1, GLUT3, GLUT1 S226D, SGLT1, SGLT2, GLUT8, GLUT8 L12A L13A, GLUT11, GLUT7, and GLUT4;
(b) modulates Krebs cycle is:
an enzyme that catalyzes a reaction in the Krebs cycle, preferably isocitrate dehydrogenase (IDH), malate dehydrogenase (MDH), or phosphoglycerate dehydrogenase (PHGDH),
an enzyme that uses a Krebs cycle metabolite as a substrate, preferably glutamic-oxaloacetic transaminase (GOT) or phosphoenolpyruvate carboxykinase 1 (PCK1), or
an enzyme that converts a precursor to a Krebs cycle metabolite, preferably phosphoserine aminotransferase (PSAT1), glutamate dehydrogenase (GDH1), glutamate-pyruvate transaminase 1 (GPT1), or glutaminase (GLS);
(c) enhances lactate concentration is:
monocarboxylate transporter (MCT), preferably MCT1, MCT2, or MCT4,
an enzyme involved in lactate synthesis, preferably lactate dehydrogenase A (LDHA), or
a polypeptide that inhibits a pathway that competes for lactate-synthesis substrates, preferably pyruvate dehydrogenase kinase 1 (PDK1), or
(d) diverts or redirects glucose metabolites out of a glycolysis pathway is selected from the group consisting of pyruvate kinase muscle isozyme M2 (PKM2), glutamine-fructose-6-phosphate aminotransferase 1 (GFPT1), and TP53-inducible glycolysis and apoptosis regulator (TIGAR).

3. The method of immune cell population for use according to claim 1, wherein the expression or overexpression of the polypeptide of any one of (a) to (d) lead to an at least 70% increase of proliferation of the immune cell population when compared to the native immune cell population of the same type.

4. The method of claim 1, wherein the immune cell population comprises immune cells selected from the group consisting of B cells, αβ T cells, γδ T cells, natural killer (NK) cells and NKT cells.

5. The method of claim 1, wherein the immune cells comprise one or more of the following features:
(I) the immune cells are autologous;
(II) the immune cells are allogeneic;
(III) the immune cells are expanded ex vivo, and
(IV) the immune cells are activated ex vivo in the presence of one or more of 4-1BB ligand, anti-4-1BB antibody, anti-CD3 antibody, anti-CD28 antibody, IL-15, anti-IL-15 receptor antibody, IL-2, IL-7, IL-12, IL-21 and K562 cells, and an engineered artificial stimulatory cell or particle prior to administration.

6. The method of claim 1, wherein
(i) the immune cells are derived from cell lines;
(ii) the immune cells are derived from peripheral blood mononuclear cells (PBMC), hematopoietic stem cells (HSCs), cord blood stem cells or induced pluripotent stem cells (iPSCs); or
(iii) the immune cells are from tumor tissue.

7. The method of claim 6, wherein the immune cells derived from (iii) are tumor-infiltrating lymphocytes (TILs).

8. The method of claim 1, wherein the immune cells are genetically engineered immune cell expressing a chimeric receptor polypeptide, wherein the chimeric receptor polypeptide comprises:
(a) an extracellular target binding domain;
(b) a transmembrane domain; and
(c) a cytoplasmic signaling domain.

9. The method of claim 8, wherein the chimeric receptor polypeptide is a chimeric antigen receptor (CAR) polypeptide or a T-Cell Receptor (TCR) polypeptide.

10. The method of claim 9, wherein the chimeric receptor polypeptide is the CAR, and wherein the extracellular target binding domain (a) is an extracellular antigen binding domain.

11. The method of claim 10, wherein the extracellular antigen binding domain is a single chain variable fragment (scFv) or a single domain antibody, and wherein the extracellular antigen binding domain binds to a tumor antigen.

12. The method of claim 8, wherein the transmembrane domain (b) is of a membrane protein selected from the group consisting of CD8α, CD8β, 4-1BB, CD28, CD34, CD4, FcεRIγ, CD16α, OX40, CD3ζ, CD3ε, CD3γ, CD3δ, TCRα, CD32, CD64, VEGFR2, FAS, NKG2D, NKp44, NKp46, and FGFR2B.

13. The method of claim 8, wherein the cytoplasmic signaling domain of (c) comprises a cytoplasmic domain of CD3 or FcεRIγ, preferably CD33.

14. The method of claim 8, wherein the chimeric receptor polypeptide comprises one or more of the following features:
(i) a signal peptide at its N-terminus;
(ii) a hinge domain, which is located at the C-terminus of (a) and the N-terminus of (b);
(iii) is free of a hinge domain;
(iv) further comprises at least one co-stimulatory signaling domain;
(v) is free of a co-stimulatory signaling domain; and
(vi) the cytoplasmic signaling domain comprises an immunoreceptor tyrosine-based activation motif (ITAM).

15. The method of claim 14, wherein the chimeric receptor polypeptide comprises the at least one co-stimulatory signaling domain (iv), which is selected from the group consisting of CD8α, CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL, or any functional variant thereof.

16. The method of claim 1, wherein the IL-2/IL-15Rβγ agonist is an IL-15/IL-15Rα complex comprising:
I. a first polypeptide comprising the amino acid sequence of the interleukin 15 (IL-15) or a derivative thereof; and
II. a second polypeptide comprising the amino acid sequence of the sushi domain of an interleukin 15-receptor alpha (IL-15Rα) or a derivative thereof.

17. The method of claim 16, wherein the first polypeptide comprises the amino acid sequence of human IL-15 or a derivative thereof,
wherein the IL-15 derivative has an amino acid sequence having a percentage of identity of at least 92.5% with the amino acid of SEQ ID NO: 74, and
wherein the second polypeptide comprises the sushi domain of the human IL-15Rα or a derivative thereof, wherein the sushi domain of the IL-15Rα derivative has an amino acid sequence at least 92% identical with the amino acid sequence of SEQ ID NO: 76, preferably at least 92% identical with the amino acid sequence of SEQ ID NO: 77.

18. The method of claim 16, the complex is a fusion protein comprising the first and second polypeptides.

19. The method of claim 18, wherein the first polypeptide is in a C-terminal position relative to the second polypeptide in the fusion protein.

20. The method of claim 16, wherein the amino acid sequence of the IL-15 or derivatives thereof and the amino acid sequence of the sushi domain of the IL-15Rα or derivatives are separated by a peptide linker.

21. The method of claim 1, wherein the agonist comprises the sequence of SEQ ID NO: 79.

22. The method of claim 1, wherein the agonist comprises:
(i) a polypeptide comprising the amino acid sequence of an IL-15 derivative having at least 0.1% of the activity of human IL-15 on the proliferation induction of kit225 cell line, and
(ii) a polypeptide comprising the amino acid sequence of the sushi domain of IL-15Rα or derivatives thereof having at least 10% of the binding activity of the sushi domain of human IL-15Rα to human IL-15.

23. The method of claim 22, wherein the IL-15 derivative has at least 25% of the activity of human IL-15 on the proliferation induction of kit225 cell line, more preferably at least 50%, and has an amino acid sequence having a percentage of identity of at least 92.5% with an amino acid sequence of SEQ ID NO: 74, preferably of at least 96%, and more preferably of at least 98.5% or of at least 99% identity.

24. The method of claim 22, wherein the derivative of the sushi domain of IL-15Rα has at least 25%, more preferably at least 50% of the binding activity of the sushi domain of human IL-15Rα, and has an amino acid sequence having a percentage of identity of at least 92.5% with an amino acid sequence selected in the group comprising SEQ ID NO: 76 or SEQ ID NO: 77, preferably of at least 96%, and more preferably of at least 98% identity.

25. The method of claim 1, wherein an effective dose of the immune cell population and an effective dose of the agonist are administered sequentially.

26. The method of claim 1, wherein the agonist and the immune cell population are administered to the subject sequentially, and wherein the agonist is administered to the subject following a cyclical regimen, wherein the cyclical regimen comprises:
(a) a first period of “x” days during which the agonist is administered at a daily dose on “y” consecutive days at the beginning of the first period followed by “x-y” days without administration of the agonist,
wherein “x” is 7, 14 or 21 days, preferably 7 days and “y” is 1, 2, 3 or 4 consecutive days;
(b) repeating the first period at least once; and
(c) a second period of “z” days without administration of the agonist wherein z is 7, 14 or, 21 days, preferably 7 or 14 days.

27. The method of claim 1, wherein the agonist is administered to the subject at a daily dose of 4.4 μmol/kg to 2200 μmol/kg, preferably 22 μmol/kg to 660 μmol/kg, and more preferably 220 pmol/kg to 530 μmol/kg.

28. The method of claim 1, wherein the administration of the agonist to the subject results in:
(a) an increase of the % of Ki-67⁺ NK of total NK cells in comparison to no administration of the agonist, and wherein the administration of the agonist after at least one repetition of the first period (step (b)) results in a Ki-67⁺ NK cell level that is at least 70% of the of the Ki-67⁺ NK cells;
(b) maintenance of NK cell numbers or preferably an increase of NK cell numbers to at least 110% as compared to no administration of agonist after at least one repetition of the first period (step (b)); and/or
(c) NK cell numbers of at least 1.1×10³ NK cells/μl after at least one repetition of the first period (step (b)).

29. The method of claim 1, wherein the immune cell population is administered to the subject at a dose of at least about 1×10⁴ immune cells/kg, preferably from about 1×10⁴ to about 1×10¹² cells/kg.

30. The method of claim 1, wherein the treatment of cancer further comprises administering to the subject:
(a) a lymphocyte reduction treatment, which optionally comprises cyclophosphamide and/or fludarabine;
(b) a therapeutic agent; and/or
(c) a further anti-tumor treatment.

31. The method of claim 1, wherein the subject is a human patient suffering from a cancer, optionally wherein the cancer is a solid tumor or liquid tumor; optionally
i. wherein the solid tumor is selected from the group consisting of carcinoma, lymphoma, sarcoma and blastoma, or
ii. wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, mesothelioma, pancreatic cancer, head and neck cancer, retinoblastoma, glioma, glioblastoma, liver cancer, and thyroid cancer; optionally wherein the cancer of B-cell origin is selected from the group consisting of Hodgkin lymphoma and non-Hodgkin lymphoma.

32. The method of claim 1, wherein the tumor has a low-glucose tumor microenvironment.

33. A method for treatment of cancer in a subject, comprising administering to a subject in need thereof a viral particle in combination with an IL-2/IL-15Rβγ agonist, wherein the viral particle is suitable for transducing an immune cell as defined in claim 1 and comprises a vector genome comprising a polynucleotide sequence encoding a chimeric receptor polypeptide,
wherein the IL-2/IL-15Rβγ agonist is an IL-15/IL-15Rα complex comprising:
I. a first polypeptide comprising the amino acid sequence of the interleukin 15 (IL-15) or a derivative thereof; and
II. a second polypeptide comprising the amino acid sequence of the sushi domain of an interleukin 15-receptor alpha (IL-15Rα) or a derivative thereof, and wherein the chimeric receptor polypeptide comprises:
(a) an extracellular target binding domain;
(b) a transmembrane domain; and
(c) a cytoplasmic signaling domain.

34. The method of claim 33, wherein the viral particle is a retroviral particle or a lentiviral particle.

35. The method of claim 33, wherein the immune cell is a T cell.

36. The method of claim 33, wherein the viral particle is for intravenous, intraperitoneal, subcutaneous or intranodal injection.

37. A method for treatment of cancer in a subject, comprising administering to a subject in need thereof a nucleic acid in combination with an Interleukin-2/Interleukin-15 receptor beta gamma (IL-2/IL-15Rβγ) agonist as defined in claim 1, wherein the nucleic acid comprises a polynucleotide sequence encoding a chimeric receptor polypeptide, which comprises:
(a) an extracellular target binding domain;
(b) a transmembrane domain; and
(c) a cytoplasmic signaling domain.

38. The method of claim 37, wherein the nucleic acid is a vector.

39. A method for treatment of cancer in a subject, comprising administering to a subject in need thereof a non-viral in vivo transduction system comprising the nucleic acid of as defined in claim 37, preferably wherein the non-viral in vivo transduction system is a lipid nanoparticle, a liposome, a polymeric nanoparticle or an inorganic nanoparticle.


SEQUENCE LISTING (99 sequences)

SEQ ID NO: 1 - protein, 19 aa, synthetic construct
ATNFSLLKQAGDVEENPGP

SEQ ID NO: 2 - protein, 18 aa, synthetic construct
EGRGSLLTCGDVEENPGP

SEQ ID NO: 3 - protein, 20 aa, synthetic construct
QCTNYALLKLAGDVESNPGP

SEQ ID NO: 4 - protein, 23 aa, synthetic construct
AVKQTLNFDLLKLAGDVESNPGP

SEQ ID NO: 5 - DNA, 463 bp, synthetic construct
  source 1..463: mol_type = other DNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 6 - protein, 25 aa, synthetic construct
DIYIWAPLAGTCGVLLLSLVITLYC

SEQ ID NO: 7 - protein, 28 aa, synthetic construct
DIISFFLALTSTALLFLLFFLTLRFSVV

SEQ ID NO: 8 - protein, 30 aa, synthetic construct
DFWVLVVVGGVLACYSLLVTVAFIIFWVRS

SEQ ID NO: 9 - protein, 25 aa, synthetic construct
DLIALVTSGALLAVLGITGYFLMNR

SEQ ID NO: 10 - protein, 25 aa, synthetic construct
DLLAALLALLAALLALLAALLARSK

SEQ ID NO: 11 - protein, 26 aa, synthetic construct
DMALIVLGGVAGLLLFIGLGIFFCVR

SEQ ID NO: 12 - protein, 25 aa, synthetic construct
DLCYILDAILFLYGIVLTLLYCRLK

SEQ ID NO: 13 - protein, 25 aa, synthetic construct
DLLLILLGVLAGVLATLAALLARSK

SEQ ID NO: 14 - protein, 25 aa, synthetic construct
DITLGLLVAGVLVLLVSLGVAIHLC

SEQ ID NO: 15 - protein, 25 aa, synthetic construct
DVSFCLVMVLLFAVDTGLYFSVKTN

SEQ ID NO: 16 - protein, 25 aa, synthetic construct
DVAAILGLGLVLGLLGPLAILLALY

SEQ ID NO: 17 - protein, 29 aa, synthetic construct
DVMSVATIVIVDICITGGLLLLVYYWSKN

SEQ ID NO: 18 - protein, 25 aa, synthetic construct
DLCYLLDGILFIYGVILTALFLRVK

SEQ ID NO: 19 - protein, 25 aa, synthetic construct
DGFLFAEIVSIFVLAVGVYFIAGQD

SEQ ID NO: 20 - protein, 26 aa, synthetic construct
DGIIVTDVIATLLLALGVFCFAGHET

SEQ ID NO: 21 - protein, 24 aa, synthetic construct
DVIGFRILLLKVAGFNLLMTLRLW

SEQ ID NO: 22 - protein, 27 aa, synthetic construct
DIIVAVVIATAVAAIVAAVVALIYCRK

SEQ ID NO: 23 - protein, 25 aa, synthetic construct
DVLFYLAVGIMFLVNTVLWVTIRKE

SEQ ID NO: 24 - protein, 25 aa, synthetic construct
DIIILVGTAVIAMFFWLLLVIILRT

SEQ ID NO: 25 - protein, 21 aa, synthetic construct
DLGWLCLLLLPIPLIVWVKRK

SEQ ID NO: 26 - protein, 25 aa, synthetic construct
DIAIYCIGVFLIACMVVTVILCRMK

SEQ ID NO: 27 - protein, 28 aa, synthetic construct
FACDIYIWAPLAGTCGVLLLSLVITLYC

SEQ ID NO: 28 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 21 aa, synthetic construct
MALPVTALLLPLALLLHAARP

SEQ ID NO: 30 - protein, 18 aa, synthetic construct
MLRLLLALNLFPSIQVTG

SEQ ID NO: 31 - protein, 20 aa, synthetic construct
METDTLLLWVLLLWVPGSTG

SEQ ID NO: 32 - protein, 44 aa, synthetic construct
TTTPAPRPPTPAPTIASQPLSLRPEACRPAAGGAVHTRGLDFAC

SEQ ID NO: 33 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 119 aa, synthetic construct
EPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 12 aa, synthetic construct
EPKSCDKTHTCP

SEQ ID NO: 36 - protein, 12 aa, synthetic construct
ESKYGPPCPPCP

SEQ ID NO: 37 - protein, 29 aa, synthetic construct
TTTPAPRPPTPAPTIASQPLSLRPEAFAC

SEQ ID NO: 38 - protein, 14 aa, synthetic construct
TTTPAPRPPTPFAC

SEQ ID NO: 39 - protein, 39 aa, synthetic construct
IEVMYPPPYLDNEKSNGTIIHVKGKHLCPSPLFPGPSKP

SEQ ID NO: 40 - protein, 39 aa, synthetic construct; identical to SEQ ID NO: 39

SEQ ID NO: 41 - protein, 26 aa, synthetic construct
KSNGTIIHVKGKHLCPSPLFPGPSKP

SEQ ID NO: 42 - protein, 16 aa, synthetic construct
GKHLCPSPLFPGPSKP

SEQ ID NO: 43 - protein, 30 aa, synthetic construct
GGGGSGGGGSGGGGSGGGGSGGGGSGGGGS

SEQ ID NO: 44 - protein, 45 aa, synthetic construct
GGGGSGGGGSGGGGSGGGGSGGGGSGGGGSGGGGSGGGGSGGGGS

SEQ ID NO: 45 - protein, 60 aa, synthetic construct
GGGGSGGGGSGGGGSGGGGSGGGGSGGGGSGGGGSGGGGSGGGGSGGGGSGGGGSGGGGS

SEQ ID NO: 46 - protein, 42 aa, synthetic construct
KRGRKKLLYIFKQPFMRPVQTTQEEDGCSCRFPEEEEGGCEL

SEQ ID NO: 47 - protein, 41 aa, synthetic construct
RSKRSRLLHSDYMNMTPRRPGPTRKHYQPYAPPRDFAAYRS

SEQ ID NO: 48 - protein, 42 aa, synthetic construct
ALYLLRRDQRLPPDAHKPPGGGSFRTPIQEEQADAHSTLAKI

SEQ ID NO: 49 - protein, 22 aa, synthetic construct
ERVQPLEENVGNAARPRFERNK

SEQ ID NO: 50 - protein, 34 aa, synthetic construct
KKKYSSSVHDPNGEYMFMRAVNTAKKSRLTDVTL

SEQ ID NO: 51 - protein, 48 aa, synthetic construct
QRRKYRSNKGESPVEPAEPCHYSCPREEEGSTIPIQEDYRKPEPACSP

SEQ ID NO: 52 - protein, 58 aa, synthetic construct
QLGLHIWQLRSQCMWPRETQLLLEVPPSTEDARSCQFPEEERGERSAEEKGRLGDLWV

SEQ ID NO: 53 - protein, 60 aa, synthetic construct
CVKRRKPRGDVVKVIVSVQRKRQEAEGEATVIEALQAPPDVTTVAVEETIPSFTGRSPNH

SEQ ID NO: 54 - protein, 48 aa, synthetic construct
KKYFFKKEVQQLSVSFSSLQIKALQNAVEKEVQAEDNIYIENSLYATD

SEQ ID NO: 55 - protein, 84 aa, synthetic construct
DIYIWAPLAGTCGVLLLSLVITLYCYKVGFFKRNLKEKMEAGRGVPNGIPAEDSEQLASGQEAGDPGCLKPLHEKDSESGGGKD

SEQ ID NO: 56 - protein, 141 aa, synthetic construct
DIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 57 - protein, 89 aa, synthetic construct
RSKRSRLLHSDYMNMTPRRPGPTRKHYQPYAPPRDFAAYRSQRRKYRSNKGESPVEPAEPCHYSCPREEEGSTIPIQEDYRKPEPACSP

SEQ ID NO: 58 - protein, 78 aa, synthetic construct
RSKRSRLLHSDYMNMTPRRPGPTRKHYQPYAPPRDFAAYRSRRDQRLPPDAHKPPGGGSFRTPIQEEQADAHSTLAKI

SEQ ID NO: 59 - protein, 83 aa, synthetic construct
KRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 60 - protein, 80 aa, synthetic construct
RSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 61 - protein, 75 aa, synthetic construct
RSKRSRLLHSDYMNMTPRRPGPTRKHYQPYAPPRDFAAYRSKKKYSSSVHDPNGEYMFMRAVNTAKKSRLTDVTL

SEQ ID NO: 62 - protein, 41 aa, synthetic construct
RSKRSRGGHSDYMNMTPRRPGPTRKHYQPYAPPRDFAAYRS

SEQ ID NO: 63 - protein, 83 aa, synthetic construct
RSKRSRGGHSDYMNMTPRRPGPTRKHYQPYAPPRDFAAYRSKRGRKKLLYIFKQPFMRPVQTTQEEDGCSCRFPEEEEGGCEL

SEQ ID NO: 64 - protein, 23 aa, synthetic construct
CARPRRSPAQEDGKVYINMPGRG

SEQ ID NO: 65 - protein, 61 aa, synthetic construct
NRRRRRERRDLFTESWDTQKAPNNYRSPISTSQPTNQSMDDTREDIYVNYPTFSRRPKTRV

SEQ ID NO: 66 - protein, 45 aa, synthetic construct
GSTVYYQGKCLTWKGPRRQLPAVVPAPLPPPCGSSAHLLPPVPGG

SEQ ID NO: 67 - protein, 63 aa, synthetic construct
WWGDIWWKTMMELRSLDTQKATCHLQQVTDLPWTSVSSPVEREILYHTVARTKISDDDDEHTL

SEQ ID NO: 68 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 69 - protein, 133 aa, synthetic construct [sequence omitted]

SEQ ID NO: 70 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 71 - protein, 100 aa, synthetic construct
PKKKIQLHAEHALYDALMILNIVKTNSPPAEEKLEDYAFNFELILEEIARLFESGDQKDEACKAKRMKEWMKRIKTTASEDEQEEMANAIITILQSWIFS

SEQ ID NO: 72 - protein, 752 aa, synthetic construct
MYRMQLLSCIALSLALVTNSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETTFHADICTLSEKERQIKKQTALVELVKHKPKATKEQLKAVMDDFAAFVEKCCKADDKETCFAEEGKKLVAASQAALGL

SEQ ID NO: 73 - protein, 160 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIN

SEQ ID NO: 74 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 120 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPE

SEQ ID NO: 76 - protein, 61 aa, synthetic construct
CPPPMSVEHADIWVKSYSLYSRERYICNSGFKRKAGTSSLTECVLNKATNVAHWTTPSLKC

SEQ ID NO: 77 - protein, 77 aa, synthetic construct
ITCPPPMSVEHADIWVKSYSLYSRERYICNSGFKRKAGTSSLTECVLNKATNVAHWTTPSLKCIRDPALVHQRPAPP

SEQ ID NO: 78 - protein, 20 aa, synthetic construct
SGGSGGGGSGGGSGGGGSGG

SEQ ID NO: 79 - protein, 211 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 20 aa, synthetic construct
METDTLLLWVLLLWVPGSTG

SEQ ID NO: 81 - protein, 297 aa, synthetic construct
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 112 aa, synthetic construct
VNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 83 - protein, 114 aa, synthetic construct
NWVNVISDLKKIEDLIQSMHIDATLYTESDVHPSCKVTAMKCFLLELQVISCESGDASIHDTVENLIILANNSLSSNGNVTESGCKECEELEEKNIKEFLQSFVHIVQMFINTS

SEQ ID NO: 84 - protein, 315 aa, synthetic construct
ITCPPPMSVEHADIWVKSYSLYSRERYICNSGFKRKAGTCSLTECVLNKATNVAHWTTPSLKCIRDPALVHQRGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 350 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 86 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 87 - protein, 461 aa, synthetic construct
MALPVTALLLPLALLLHAARPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 913 aa, synthetic construct
MALPVTALLLPLALLLHAARPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 89 - protein, 487 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 483 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 430 aa, synthetic construct
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 92 - protein, 699 aa, synthetic construct
MCGIFAYLNYHVPRTRREILETLIKGLQRLEYRGYDSAGVGFDGGNDKDWEANACKIQLIKKKGKVKALDEEVHKQQDMDLDIEFDVHLGIAHTRWATHGEPSPVNSHPQRSDKNNEFIVIHNGIITNYKDLKKFLESKGYDFESETDTETIAKLVKYMYDNRESQDTSFTTLVERVIQQLEGAFALVFKSVHFPGQAVGTRRGSPLLIGVRSEHKLSTDHIPILYRTARTQIGSKFTRWGSQGERGKDKKGSCNLSRVDSTTCLFPVEEKAVEYYFASDASAVIEHTNRVIFLEDDDVAAVVDGRLSIHRIKRTAGDHPGRAVQTLQMELQQIMKGNFSSFMQKEIFEQPESVVNTMRGRVNFDDYTVNLGGLKDHIKEIQRCRRLILIACGTSYHAGVATRQVLEELTELPVMVELASDFLDRNTPVFRDDVCFFLSQSGETADTLMGLRYCKERGALTVGITNTVGSSISRETDCGVHINAGPEIGVASTKAYTSQFVSLVMFALMMCDDRISMQERRKEIMLGLKRLPDLIKEVLSMDDEIQKLATELYHQKSVLIMGRGYHYATCLEGALKIKEITYMHSEGILAGELKHGPLALVDKLMPVIMIIMRDHTYAKCQNALQQVVARQGRPVVICDKEDTETIKNTKRTIKVPHSVDCLQGILSVIPLQLLAFHLAVLRGYDVDFPRNLAKSVTVE

SEQ ID NO: 93 - protein, 492 aa, synthetic construct
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 94 - protein, 270 aa, synthetic construct
MARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 95 - protein, 332 aa, synthetic construct
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 96 - protein, 456 aa, synthetic construct
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYERPRRTWLQVSSLCCMACKMIFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

SEQ ID NO: 97 - protein, 412 aa, synthetic construct
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 98 - protein, 225 aa, synthetic construct
MVSHSELRKLFYSADAVCFDVDSTVIREEGIDELAKICGVEDAVSEMTRRAMGGAVPFKAALTERLALIQPSREQVQRLIAEQPPHLTPGIRELVSRLQERNVQVFLISGGFRSIVEHVASKLNIPATNVFANRLKFYFNGEYAGFDETQPTAESGGKGKVIKLLKEKFHFKKIIMIGDGATDMEACPPADAFIGFGGNVIRQQVKDNAKWYITDFVELLGELEE

SEQ ID NO: 99 - protein, 405 aa, synthetic construct
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS